(19) United States
(12) Patent Application Publication
(10) Pub. No.: US 2025/0057827 A1
(43) Pub. Date: Feb. 20, 2025

(54) CYTOTOXICITY TARGETING CHIMERAS FOR PROSTATE SPECIFIC MEMBRANE ANTIGEN-EXPRESSING CELLS

(71) Applicant: GLAXOSMITHKLINE INTELLECTUAL PROPERTY DEVELOPMENT LIMITED, Brentford (GB)

(72) Inventors: Christina Ng Di Marco, Collegeville, PA (US); Matthew Robert Sender, Collegeville, PA (US); Brandon James Turunen, Boston, MA (US)

(21) Appl. No.: 18/808,401

(22) Filed: Aug. 19, 2024

Related U.S. Application Data
(63) Continuation of application No. PCT/IB2023/051744, filed on Feb. 24, 2023.
(60) Provisional application No. 63/313,845, filed on Feb. 25, 2022.

(51) Int. Cl. A61K 31/4439; C07D 401/04; C07K 16/16

(52) U.S. Cl. CPC A61K 31/4439; C07D 401/04; C07K 16/16; C07K 2317/565; C07K 2317/732

(57) ABSTRACT

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer.

### CROSS-REFERENCE TO RELATED APPLICATIONS

This application is a continuation of International Patent Application No. PCT/IB2023/051744, filed Feb. 24, 2023, which claims priority to U.S. Patent Application No. 63/313,845 filed on Feb. 25, 2022; the contents of each of which are incorporated by reference herein in their entirety.

### REFERENCE TO AN ELECTRONIC SEQUENCE LISTING

This application contains a Sequence Listing which has been submitted electronically via Patent Center in XML format and is hereby incorporated by reference in its entirety. Said XML copy, created on Aug. 16, 2024, is named 211806_seqlist.xml and is 20,577 bytes in size.

### FIELD OF THE DISCLOSURE

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antibody protein. The present disclosure also relates to agents capable of binding to a receptor on a surface of a pathogenic cell and inducing the depletion of the pathogenic cell in a subject for use in the treatment of cancer.

### BACKGROUND

Cell-surface proteins and their ligands play key roles in tumor initiation, growth and metastasis. Antibody-based therapeutics have promising properties as drug candidates for these indications due to their selectivity for pathogenic cell-surface targets and their ability to direct immune surveillance to target-expressing tissues or cells to induce depletion of the pathogenic cells. Examples of such depletion mechanisms include antibody-dependent cellular cytotoxicity (ADCC), antibody-dependent cellular phagocytosis (ADCP), and complement-dependent cytotoxicity (CDC). However, antibody-based therapeutics often suffer from a lack of bioavailability, high cost, thermal instability, and difficult manufacturing due to their size, complexity and peptide based structures. Conversely, small molecule therapeutics often provide affordability, stability, and the convenience of oral dosing, but may suffer from poor selectivity and off-target effects, while also lacking the immune control of therapeutic antibodies.

Accordingly, a need exists for improved therapeutic approaches that target pathogenic cells for use in the treatment of disease. Such compositions and related methods are provided in the present disclosure.

### SUMMARY

In one aspect, the present disclosure provides a heterobifunctional molecule referred to as a cytoxicity targeting chimera (CyTaC) or an antibody recruiting molecule (ARM), wherein the ARM comprises a moiety that binds a target cell-surface protein on a cell and a moiety that binds an exogenous antibody. In a further aspect, the ARM comprises a divalent linker that links the target-binding moiety to the antibody-binding moiety. In a further aspect, the target-binding moiety is prostate specific membrane antigen (PSMA)-binding moiety. In a further aspect, the exogenous antibody is an anti-cotinine antibody, or antigen-binding fragment thereof.

In a further aspect, the ARM is a compound of Formula (I):

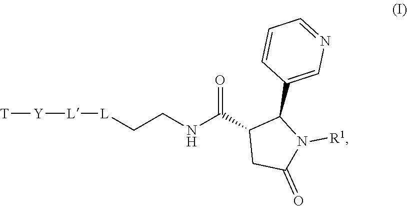

or a pharmaceutically acceptable salt thereof,
wherein:

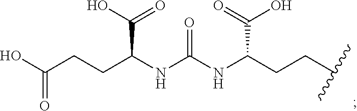 [SMILES: *CC[C@H](NC(=O)N[C@@H](CCC(=O)O)C(=O)O)C(=O)O]

### T is;

R¹ is C₁₋₄ alkyl or C₃₋₆ cycloalkyl;

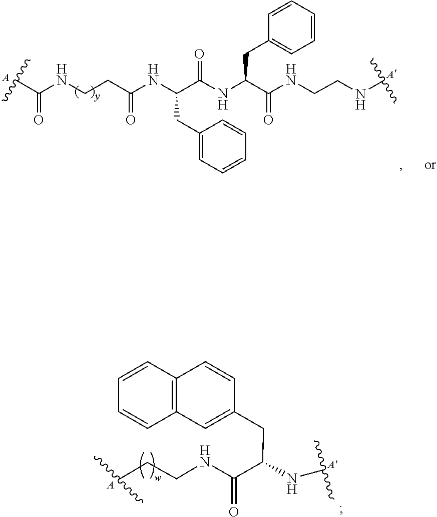

L′ is a bond,
y is an integer of 1 to 9;
w is an integer of 0 to 5;
Y is a bond or a divalent spacer moiety of one to twelve atoms in length; and
L is a divalent linker as described herein;
wherein each

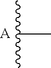

represents a covalent bond to the Y group of Formula (I), or when Y is a bond, a covalent bond to the T group of Formula (I), and each

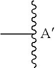

represents a covalent bond to the L group of Formula (I).

In a further aspect, the ARM is a compound of Formula (II):

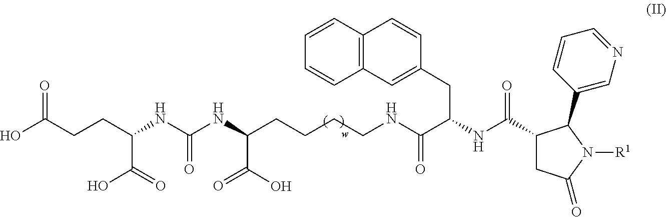

or a pharmaceutically acceptable salt thereof,
wherein:
R¹ is C₁₋₄ alkyl or C₃₋₆ cycloalkyl; and
w is an integer of 0 to 5.

In one aspect, the present disclosure provides a method of treating and/or preventing a disease or disorder in a patient in need thereof, comprising: administering to the patient a compound of Formula (I) as disclosed herein and an anti-cotinine antibody, or antigen-binding fragment thereof.

In one aspect, the present disclosure provides a method of increasing antibody-dependent cell cytotoxicity (ADCC) of PSMA-expressing cells comprising: contacting the cells with a compound of Formula (I) as disclosed herein and an anti-cotinine antibody, or antigen-binding fragment thereof.

In one aspect, the present disclosure provides a method of depleting PSMA-expressing cells comprising: contacting the cells with a compound of Formula (I) as disclosed herein and an anti-cotinine antibody, or antigen-binding fragment thereof.

In one aspect, the present disclosure provides a compound of Formula (I) as disclosed herein for use in therapy. In a further aspect, the present disclosure provides a combination comprising a compound of Formula (I) as disclosed herein and an anti-cotinine antibody, or antigen-binding fragment thereof, for use in therapy.

In one aspect, the present disclosure provides a combination comprising a compound of Formula (I) as disclosed herein and an anti-cotinine antibody, or antigen-binding fragment thereof, for use in the treatment of a disease or disorder.

In one aspect, the present disclosure provides use of a compound of Formula (I) as disclosed herein in the manufacture of a medicament for the treatment of a disease or disorder. In a further aspect, the present disclosure provides use of a combination comprising a compound of Formula (I) as disclosed herein and an anti-cotinine antibody, or antigen-binding fragment thereof, in the manufacture of a medicament for the treatment of a disease or disorder.

In one aspect, the present disclosure provides a combination comprising a compound of Formula (I) as disclosed herein and an anti-cotinine antibody, or antigen-binding fragment thereof.

### BRIEF DESCRIPTION OF THE FIGURES

FIG. 1: Schematic representation of cytotoxicity targeting chimeras (CyTaCs) technology compared to current antibody technology.

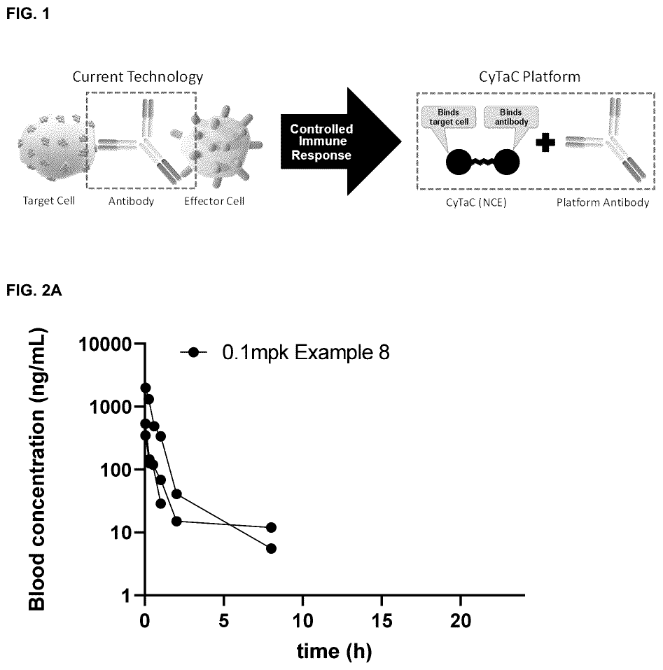

FIG. 2A, FIG. 2B, and FIG. 2C: PK analysis of compounds of Formula (I) in mice as described in Example 12; FIG. 2A shows PK analysis of the compound of Example 8 in the absence of anti-cotinine antibody; FIG. 2B shows PK analysis of the compound of Example 8 dosed in the presence of anti-cotinine antibody; FIG. 2C shows PK analysis of the compound of Example 9 dosed in the presence of anti-cotinine antibody.

### DETAILED DESCRIPTION

In one aspect, the present disclosure provides a compound of Formula (I):

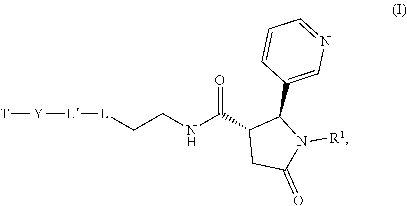

or a pharmaceutically acceptable salt thereof,
wherein:

### T is

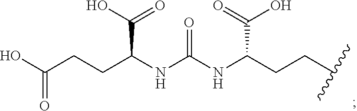 [SMILES: *CC[C@H](NC(=O)N[C@@H](CCC(=O)O)C(=O)O)C(=O)O]

R¹ is C₁₋₄ alkyl or C₃₋₆ cycloalkyl;
L′ is a bond,

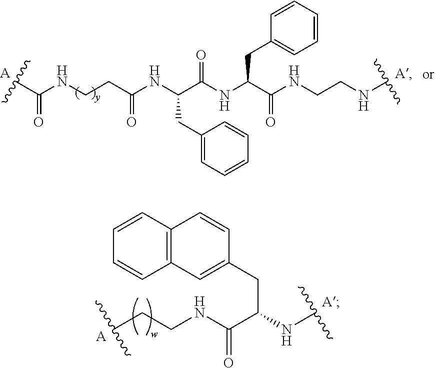

y is an integer of 1 to 9;
w is an integer of 0 to 5;
Y is a bond or a divalent spacer moiety of one to twelve atoms in length; and
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), (L-n-iv), or (L-p);
wherein each

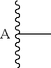

represents a covalent bond to the Y group of Formula (I), or when Y is a bond, a covalent bond to the T group of Formula (I), and each

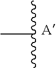

represents a covalent bond to the L group of Formula (I).

In one embodiment of the disclosure L is a divalent linker of Formula (L-a):

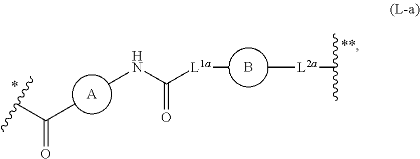

or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

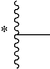

represents a covalent bond to the L′ group of Formula (I), or when L′ is a bond, a covalent bond to the Y group of Formula (I), or when both L′ and Y are a bond, a covalent bond
to the T group of Formula (I), and

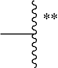

represents a covalent bond to the methylene group of Formula (I).

In another embodiment, Ring A and Ring B of Formula (L-a) are each independently

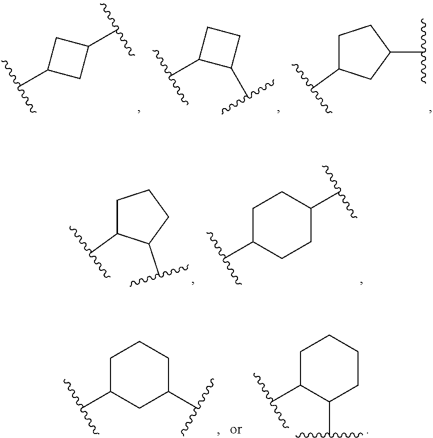

In another embodiment, L is a divalent linker of Formula (L-a-i):

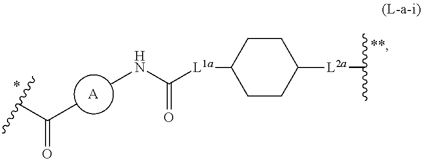

or a stereoisomer thereof,
wherein:
Ring A is C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

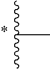

represents a covalent bond to the L′ group of Formula (I), or when L′ is a bond, a covalent bond to the Y group of Formula (I), or when both L′ and Y are a bond, a covalent bond to the T group of Formula (I), and

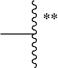

represents a covalent bond to the methylene group of Formula (I).

In another embodiment, Ring A of Formula (L-a-i) is

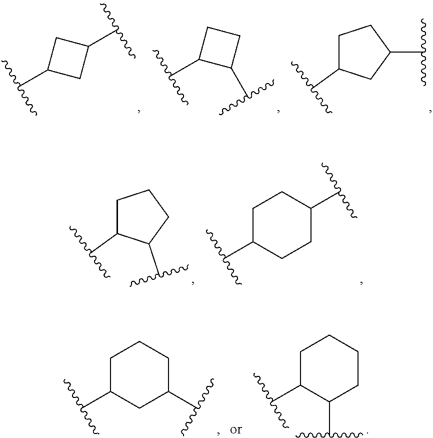

In another embodiment, L is a divalent linker of Formula (L-a-ii):

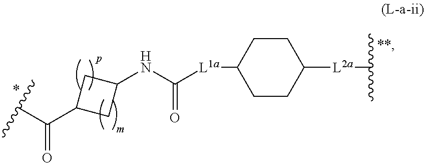

or a stereoisomer thereof,
wherein:
L^{1a} is C₃₋₅ linear alkylene, wherein 1 or 2 methylene units are replaced with —O— or —NR^{a}—;
each R^{a} is independently hydrogen or C₁₋₃ alkyl;
L^{2a} is —O—, —NHC(O)—, or —CH₂—O—;
p is 1 or 2; and
m is 1 or 2;
wherein

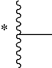

represents a covalent bond to the L′ group of Formula (I), or when L′ is a bond, a covalent bond to the Y group of Formula (I), or when both L′ and Y are a bond, a covalent bond to the T group of Formula (I), and

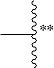

represents a covalent bond to the methylene group of Formula (I).

In another embodiment, L^{1a} of Formula (L-a), (L-a-i), or (L-a-ii) is selected from

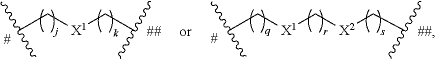

wherein:
j is 1, 2, 3, or 4;
k is 0, 1, 2, or 3;
the sum of j and k is 2, 3, or 4;
q is 1 or 2;
r is 1 or 2;
s is 0 or 1;
the sum of q, r, and s is 2 or 3;
X¹ and X² are independently —O— or NR^{a}; and
each R^{a} is independently hydrogen or C₁₋₃ alkyl;
wherein

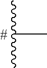

represents a covalent bond to the C(O) group of Formula (L-a), (L-a-i), or (L-a-ii), and

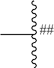

represents a covalent bond to Ring B of Formula (L-a) or to the cyclohexylene group of Formula (L-a-i) or (L-a-ii).

In another embodiment, L^{1a} of Formula (L-a), (L-a-i), or (L-a-ii) is selected from —(CH₂)₂O—, —(CH₂)₃O—, —(CH₂)₄O—, —(CH₂)₂OCH₂—, —(CH₂)₃OCH₂—, —(CH₂)₂O(CH₂)₂—, —CH₂OCH₂—, —CH₂O(CH₂)₂—, —CH₂O(CH₂)₃—, —CH₂OCH₂O—, or —CH₂OCH₂OCH₂—. In another embodiment, L^{1a} of Formula (L-a), (L-a-i), or (L-a-ii) is selected from —(CH₂)₂O—, —(CH₂)₃O—, —(CH₂)₂OCH₂—, or —(CH₂)₃OCH₂—. In another embodiment, L^{1a} of Formula (L-a), (L-a-i), or (L-a-ii) is selected from —(CH₂)₂NR^{a}—, —(CH₂)₃NR^{a}—, —(CH₂)₄NR^{a}—, —(CH₂)₂NR^{a}CH₂—, —(CH₂)₃NR^{a}CH₂—, —(CH₂)₂NR^{a}(CH₂)₂—, —CH₂NR^{a}CH₂—, —CH₂NR^{a}(CH₂)₂—, —CH₂NR^{a}(CH₂)₃—, —CH₂NR^{a}CH₂NR^{a}—, or —CH₂NR^{a}CH₂NR^{a}CH₂—, wherein each R^{a} is independently hydrogen or C₁₋₃ alkyl. In another embodiment, L^{1a} of Formula (L-a), (L-a-i), or (L-a-ii) is selected from —(CH₂)₂NR^{a}—, —(CH₂)₃NR^{a}—, —(CH₂)₂NR^{a}CH₂—, or —(CH₂)₃NR^{a}CH₂—, wherein R^{a} is hydrogen or C₁₋₃ alkyl. In another embodiment, L^{1a} of Formula (L-a), (L-a-i), or (L-a-ii) is selected from —(CH₂)₂NH—, —(CH₂)₃NH—, —(CH₂)₄NH—, —(CH₂)₂NHCH₂—, —(CH₂)₃NHCH₂—, —(CH₂)₂NH(CH₂)₂—, —CH₂NHCH₂—, —CH₂NH(CH₂)₂—, —CH₂NH(CH₂)₃—, —CH₂NHCH₂NH—, or —CH₂NHCH₂NHCH₂—. In another embodiment, L^{1a} of Formula (L-a), (L-a-i), or (L-a-ii) is selected from —(CH₂)₂NH—, —(CH₂)₃NH—, —(CH₂)₂NHCH₂—, or —(CH₂)₃NHCH₂—. In another embodiment, L^{1a} of Formula (L-a), (L-a-i), or (L-a-ii) is selected from —CH₂OCH₂NR^{a}—, —CH₂NR^{a}CH₂O—, —CH₂OCH₂NR^{a}CH₂—, —CH₂NR^{a}CH₂OCH₂—, wherein R^{a} is independently hydrogen or C₁₋₃ alkyl. In another embodiment, L^{1a} of Formula (L-a), (L-a-i), or (L-a-ii) is selected from —CH₂OCH₂NH—, —CH₂NHCH₂O—, —CH₂OCH₂NHCH₂—, —CH₂NHCH₂OCH₂—.

In another embodiment, L is a divalent linker of Formula (L-a-iii):

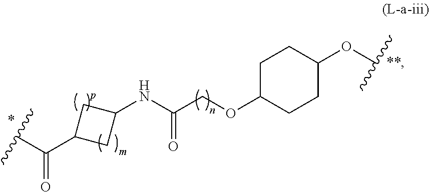

or a stereoisomer thereof,
wherein:
p is 1 or 2;
m is 1 or 2; and
n is 1, 2, or 3;
wherein

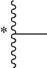

represents a covalent bond to the L′ group of Formula (I), or when L′ is a bond, a covalent bond to the Y group of Formula (I), or when both L′ and Y are a bond, a covalent bond to the T group of Formula (I), and

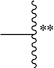

represents a covalent bond to the methylene group of Formula (I).

In another embodiment, L is a divalent linker of Formula (L-a) selected from the

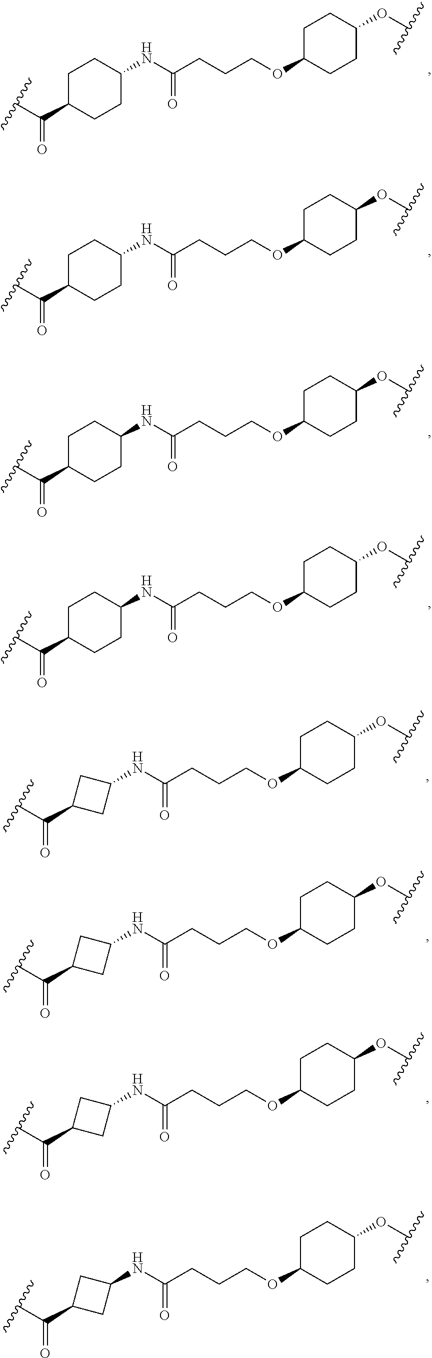

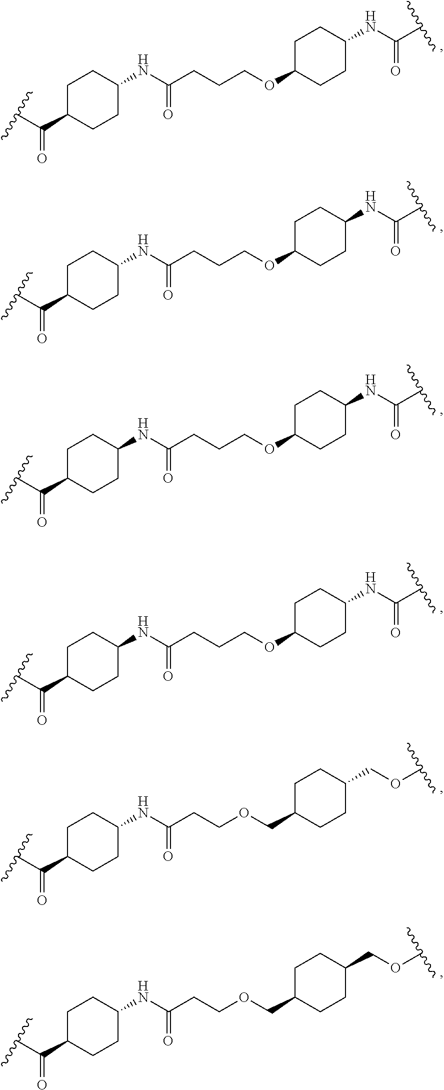 [SMILES: *C(=O)N[C@H]1CC[C@H](OCCCC(=O)N[C@H]2CC[C@H](C(*)=O)CC2)CC1 ; *C(=O)N[C@H]1CC[C@@H](OCCCC(=O)N[C@H]2CC[C@H](C(*)=O)CC2)CC1 ; *C(=O)N[C@H]1CC[C@@H](OCCCC(=O)N[C@H]2CC[C@@H](C(*)=O)CC2)CC1 ; *C(=O)N[C@H]1CC[C@H](OCCCC(=O)N[C@H]2CC[C@@H](C(*)=O)CC2)CC1 ; *OC[C@H]1CC[C@H](COCCC(=O)N[C@H]2CC[C@H](C(*)=O)CC2)CC1 ; *OC[C@H]1CC[C@@H](COCCC(=O)N[C@H]2CC[C@H](C(*)=O)CC2)CC1]

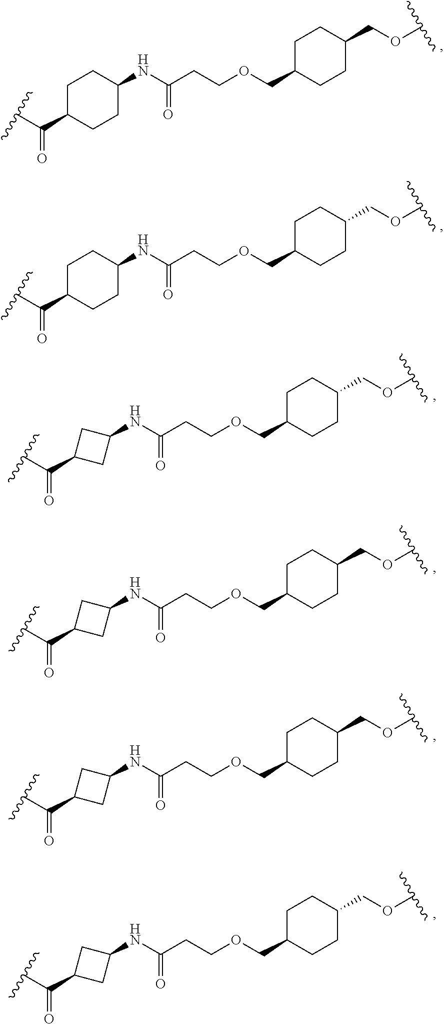 [SMILES: *OC[C@H]1CC[C@@H](COCCC(=O)N[C@H]2CC[C@@H](C(*)=O)CC2)CC1 ; *OC[C@H]1CC[C@H](COCCC(=O)N[C@H]2CC[C@@H](C(*)=O)CC2)CC1 ; *OC[C@H]1CC[C@H](COCCC(=O)N[C@H]2C[C@@H](C(*)=O)C2)CC1 ; *OC[C@H]1CC[C@@H](COCCC(=O)N[C@H]2C[C@@H](C(*)=O)C2)CC1 ; *OC[C@H]1CC[C@@H](COCCC(=O)N[C@H]2C[C@@H](C(*)=O)C2)CC1 ; *OC[C@H]1CC[C@H](COCCC(=O)N[C@H]2C[C@@H](C(*)=O)C2)CC1]

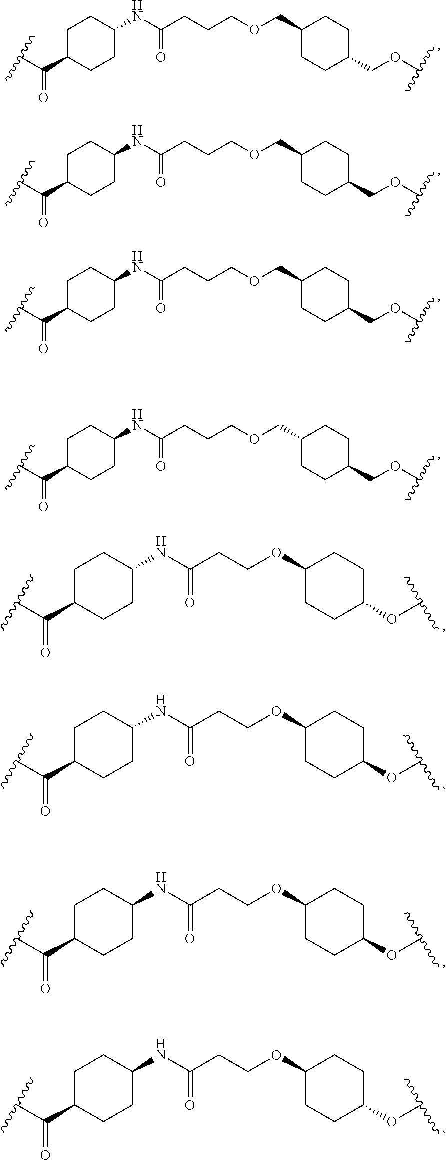

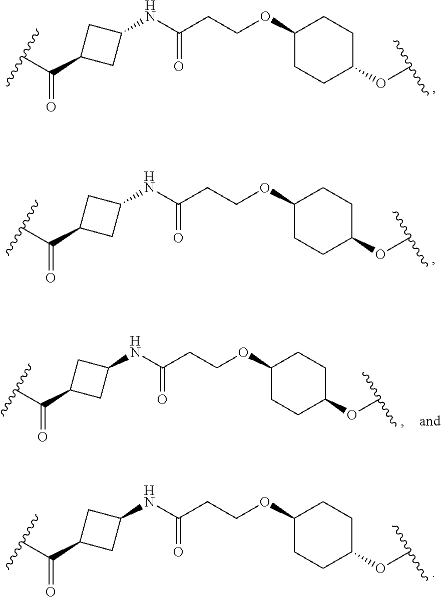 [SMILES: *O[C@H]1CC[C@H](OCCC(=O)N[C@H]2C[C@H](C(*)=O)C2)CC1 ; *O[C@H]1CC[C@@H](OCCC(=O)N[C@H]2C[C@H](C(*)=O)C2)CC1 ; *O[C@H]1CC[C@@H](OCCC(=O)N[C@H]2C[C@@H](C(*)=O)C2)CC1 ; *O[C@H]1CC[C@H](OCCC(=O)N[C@H]2C[C@@H](C(*)=O)C2)CC1]

In another embodiment, L is a divalent linker of Formula (L-b):

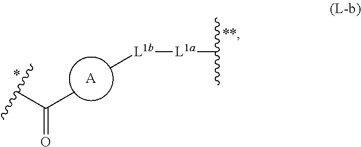

or a stereoisomer thereof,
wherein:
Ring A is C₄₋₆ cycloalkylene or C₇₋₉ bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is

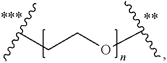

wherein n is 1, 2, 3, or 4, and

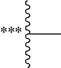

represents a covalent bond to L^{1b};
and
each R^{1b} is independently hydrogen or C₁₋₃ alkyl;
wherein

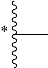

represents a covalent bond to the L′ group of Formula (I), or when L′ is a bond, a covalent bond to the Y group of Formula (I), or when both L′ and Y are a bond, a covalent bond to the T group of Formula (I), and

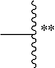

represents a covalent bond to the methylene group of Formula (I).

In another embodiment, Ring A of Formula (L-b) is

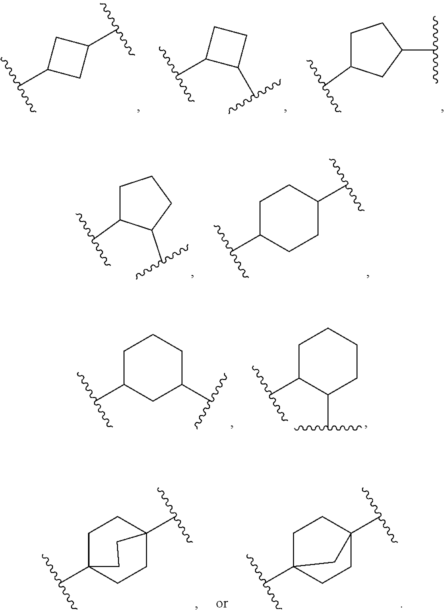

In another embodiment, L is a divalent linker of Formula (L-b-i):

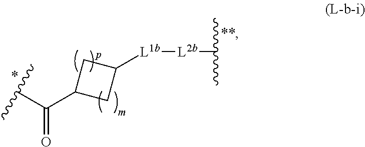

or a stereoisomer thereof,
wherein:
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is

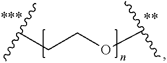

wherein n is 1, 2, 3, or 4, and

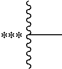

represents a covalent bond to L^{1b};
each R^{1b} is independently hydrogen or C₁₋₃ alkyl;
p is 1 or 2; and
m is 1 or 2;
wherein

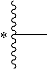

represents a covalent bond to the L′ group of Formula (I), or when L′ is a bond, a covalent bond to the Y group of Formula (I), or when both L′ and Y are a bond, a covalent bond to the T group of Formula (I), and

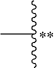

represents a covalent bond to the methylene group of Formula (I).

In another embodiment, L^{2b} of Formula (L-b) or (L-b-i) is selected from

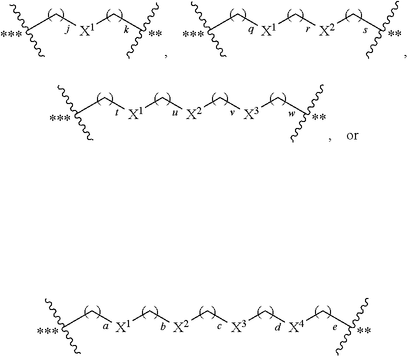

wherein:
j is 1, 2, 3, 4, 5, 6, 7, 8, 9, or 10;
k is 1, 2, 3, 4, 5, 6, 7, 8, 9, or 10;
the sum of j and k is 5, 6, 7, 8, 9, 10, or 11;
q is 1, 2, 3, 4, 5, 6, 7, 8, or 9;
r is 1, 2, 3, 4, 5, 6, 7, 8, or 9;
s is 0, 1, 2, 3, 4, 5, 6, 7, or 8;
the sum of q, r, and s is 4, 5, 6, 7, 8, 9, or 10;
t is 1, 2, 3, 4, 5, 6, or 7;
u is 1, 2, 3, 4, 5, 6, or 7;
vis 1, 2, 3, 4, 5, 6, or 7;
w is 0, 1, 2, 3, 4, 5, or 6;
the sum of t, u, v, and w is 3, 4, 5, 6, 7, 8, or 9;
a is 1, 2, 3, 4, or 5;
b is 1, 2, 3, 4, or 5;
c is 1, 2, 3, 4, or 5;
d is 1, 2, 3, 4, or 5;
e is 0, 1, 2, 3, or 4;
the sum of a, b, c, d, and e is 4, 5, 6, 7, or 8;
X¹, X², X³, and X⁴ are independently —O—, —NR^{1b}—, —C(O)NR^{1b}—, or —NR^{1b}C(O)—; and
each R^{1b} is independently hydrogen or C₁₋₃ alkyl;
wherein

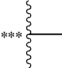

represents a covalent bond to L^{1b} of Formula (L-b) or (L-b-i), and

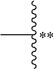

represents a covalent bond to the methylene group of Formula (I).

In another embodiment, L is a divalent linker of Formula (L-b) selected from the group consisting of:

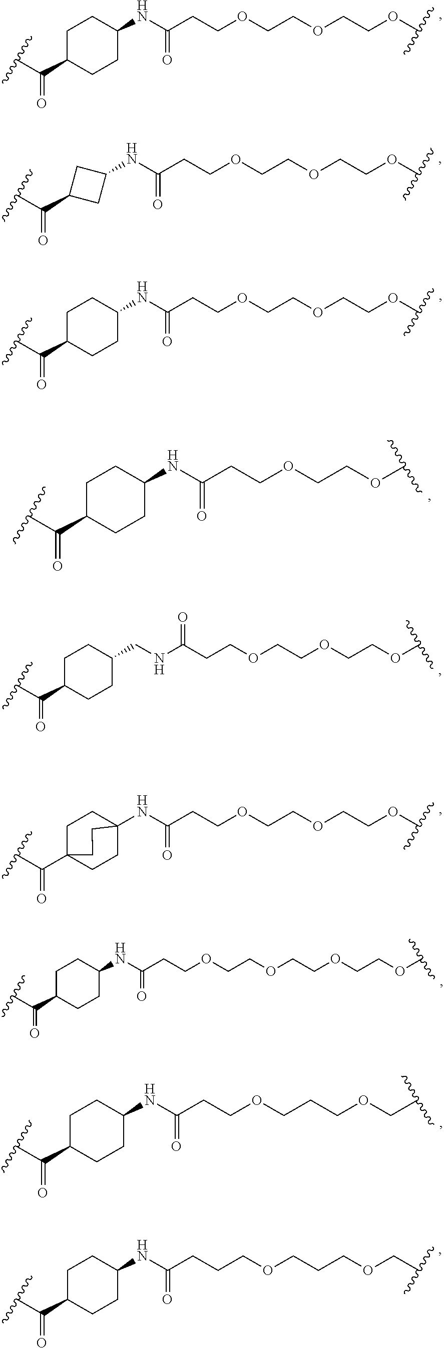

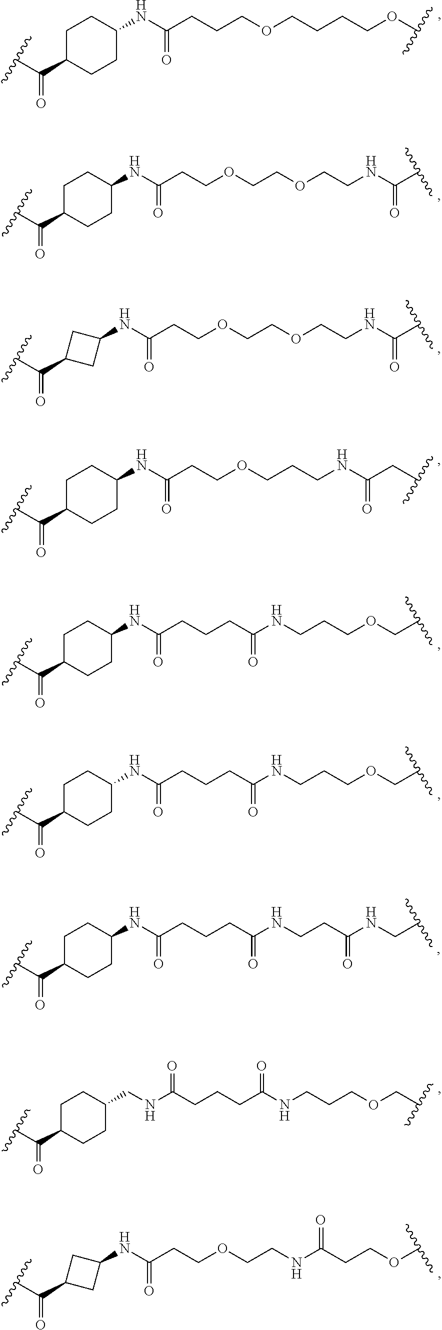

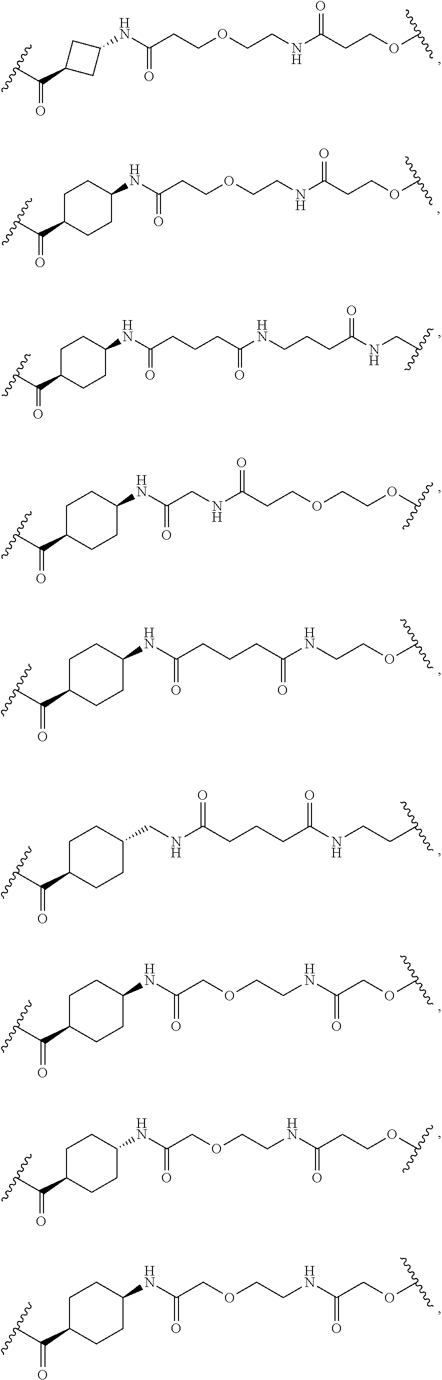

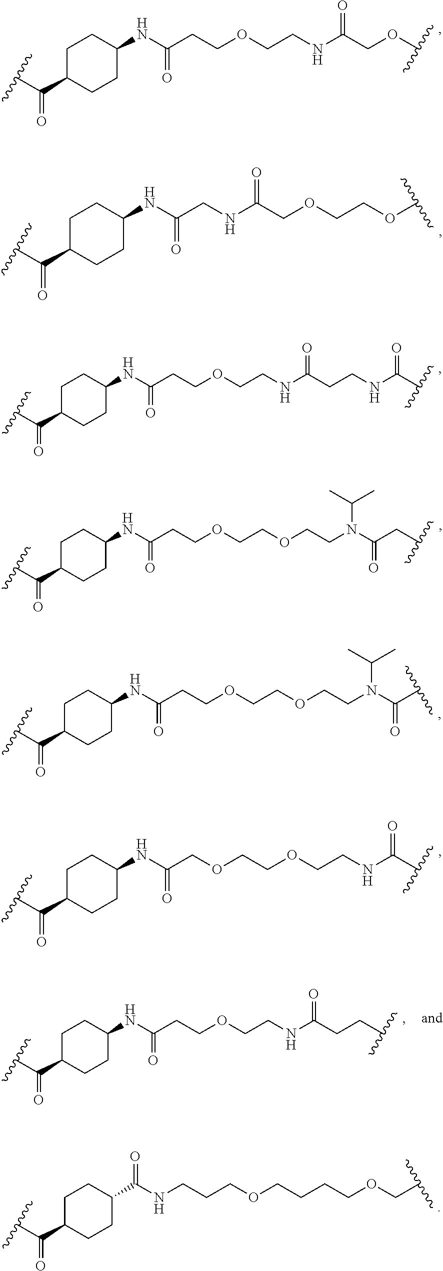

In another embodiment, L is a divalent linker of Formula (L-c):

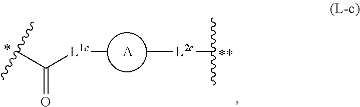

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇₋₉ bridged bicyclic cycloalkylene; and
L^{2c} is —O— or a saturated C₂₋₁₀ linear alkylene, wherein 1, 2, or 3 methylene units are replaced with —O—, —NH—, —NHC(O)—, or —C(O)NH—;
wherein

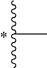

represents a covalent bond to the L′ group of Formula (I), or when L′ is a bond, a covalent bond to the Y group of Formula (I), or when both L′ and Y are a bond, a covalent bond to the T group of Formula (I), and

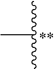

represents a covalent bond to the methylene group of Formula (I).

In another embodiment, Ring A of Formula (L-c) is

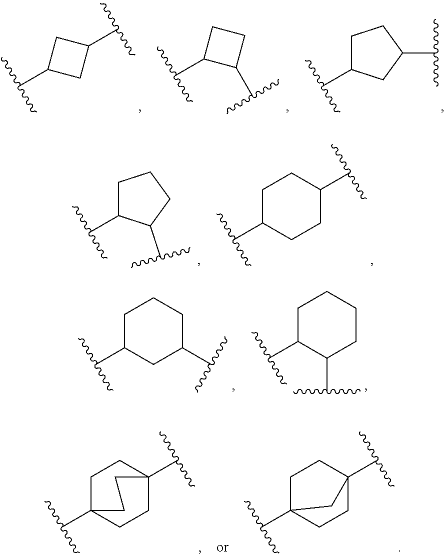

In another embodiment, L is a divalent linker of Formula (L-c-i):

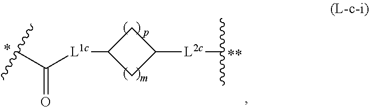

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
L^{2c} is —O— or a saturated C₂₋₁₀ linear alkylene, wherein 1, 2, or 3 methylene units are replaced with —O—, —NH—, —NHC(O)—, or —C(O)NH—;
p is 1 or 2; and
m is 1 or 2;
wherein

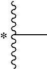

represents a covalent bond to the L′ group of Formula (I), or when L′ is a bond, a covalent bond to the Y group of Formula (I), or when both L′ and Y are a bond, a covalent bond to the T group of Formula (I), and

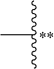

represents a covalent bond to the methylene group of Formula (I).

In another embodiment, L^{1c} of Formula (L-c) or (L-c-i) is selected from

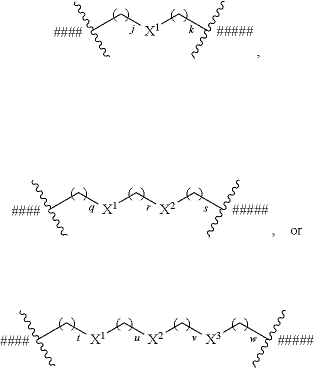

wherein:
j is 1, 2, 3, 4, 5, 6, 7, 8, or 9;
k is 0, 1, 2, 3, 4, 5, 6, 7, or 8;
the sum of j and k is 1, 2, 3, 4, 5, 6, 7, 8, or 9;
q is 1, 2, 3, 4, 5, 6, or 7;
r is 1, 2, 3, 4, 5, 6, or 7;
s is 0, 1, 2, 3, 4, 5, or 6;
the sum of q, r, and s is 2, 3, 4, 5, 6, 7, or 8;
t is 1, 2, 3, 4, or 5;
u is 1, 2, 3, 4, or 5;
v is 1, 2, 3, 4, or 5;
w is 0, 1, 2, 3, or 4;
the sum of t, u, v, and w is 3, 4, 5, 6, or 7; and
X¹, X² and X³ are independently —O—, —NH—, —NHC(O)—, or —C(O)NH—;
wherein

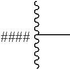

represents a covalent bond to the C(O) group of Formula (L-c) or (L-c-i), and

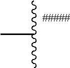

represents a covalent bond to the ring of Formula (L-c) or (L-c-i).

In another embodiment, L^{2c} of Formula (L-c) or (L-c-i) is selected from

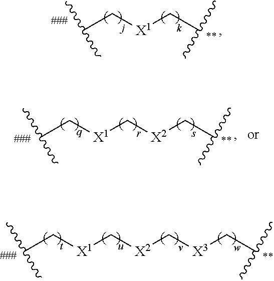

wherein:
j is 0, 1, 2, 3, 4, 5, 6, 7, 8, or 9;
k is 0, 1, 2, 3, 4, 5, 6, 7, 8, or 9;
the sum of j and k is 1, 2, 3, 4, 5, 6, 7, 8, or 9;
q is 0, 2, 3, 4, 5, 6, or 7;
r is 1, 2, 3, 4, 5, 6, 7, or 8;
s is 0, 1, 2, 3, 4, 5, 6, or 7;
the sum of q, r, and s is 1, 2, 3, 4, 5, 6, 7, or 8;
t is 0, 1, 2, 3, 4, or 5;
u is 1, 2, 3, 4, 5, or 6;
v is 1, 2, 3, 4, 5, or 6;
w is 0, 1, 2, 3, 4, or 5;
the sum of t, u, v, and w is 2, 3, 4, 5, 6, or 7; and
X¹, X² and X³ are independently —O—, —NH—, —NHC(O)—, or —C(O)NH—;
wherein

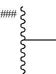

represents a covalent bond to the ring of Formula (L-c) or (L-c-i), and

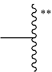

represents a covalent bond to the methylene group of Formula (I).

In another embodiment, L is a divalent linker of Formula (L-c) selected from the

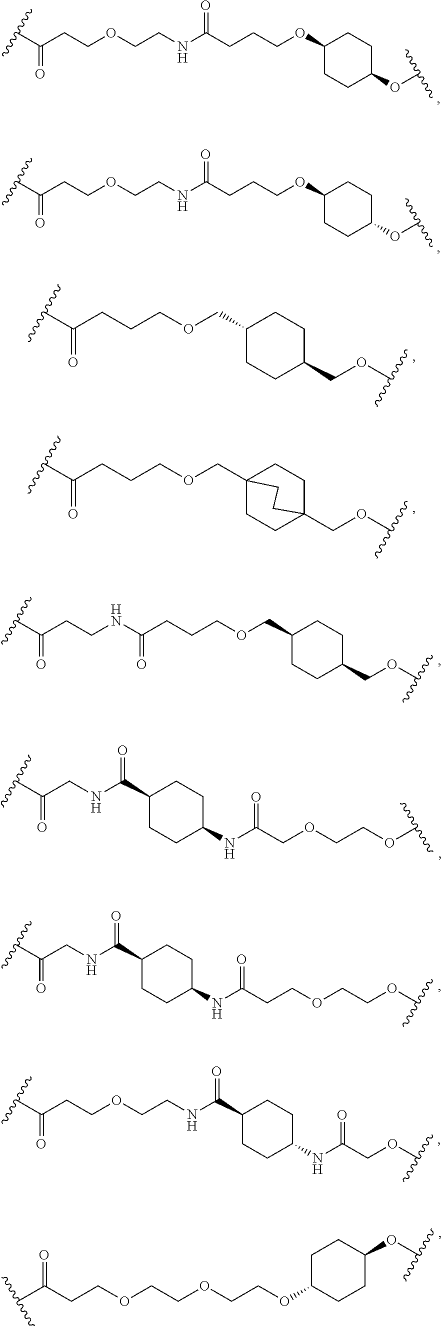

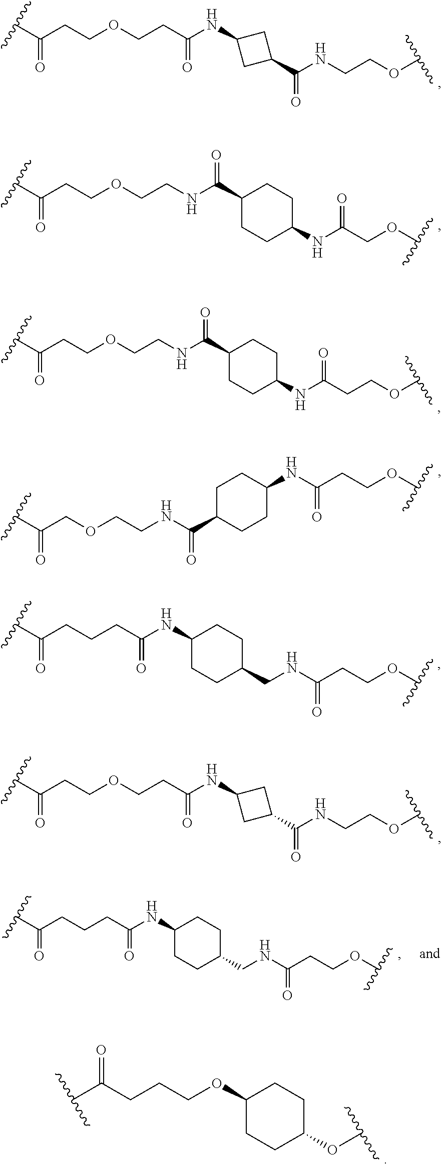

In another embodiment, L is a divalent linker of Formula (L-d):

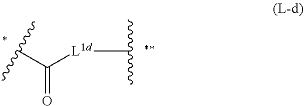

wherein:
L^{1d} is C₁₂₋₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—;
wherein

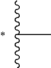

represents a covalent bond to the L′ group of Formula (I), or when L′ is a bond, a covalent bond to the Y group of Formula (I), or when both L′ and Y are a bond, a covalent bond to the T group of Formula (I), and

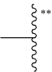

represents a covalent bond to the methylene group of Formula (I). In another embodiment, L^{1d} is a C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, or C₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—. In another embodiment, L^{1d} is C₁₂₋₂₂ linear alkylene, for example, C₁₂, C₁₃, C₁₄, C₁₅, C₁₆, C₁₇, C₁₈, C₁₉, C₂₀, C₂₁, or C₂₂, wherein 1, 2, 3, 4, or 5 methylene units are replaced with —NH—, —O—, —C(O)NH—, —NHC(O)—, or —NHC(O)—NH—.

In another embodiment, L^{1d} of Formula (L-d) is selected from

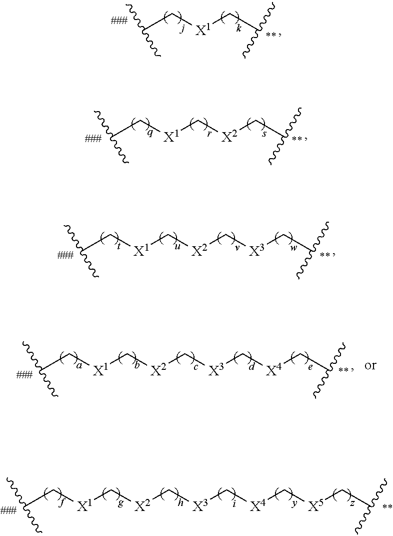

wherein:
j is 1, 2, 3, 4, 5, 6, 7, 8, 9, 10, 11, 12, 13, 14, 15, 16, 17, 18, 19, or 20;
k is 1, 2, 3, 4, 5, 6, 7, 8, 9, 10, 11, 12, 13, 14, 15, 16, 17, 18, 19, or 20;
the sum of j and k is 11, 12, 13, 14, 15, 16, 17, 18, 19, 20, or 21;
q is 1, 2, 3, 4, 5, 6, 7, 8, 9, 10, 11, 12, 13, 14, 15, 16, 17, 18, or 19;
r is 1, 2, 3, 4, 5, 6, 7, 8, 9, 10, 11, 12, 13, 14, 15, 16, 17, 18, or 19;
s is 0, 1, 2, 3, 4, 5, 6, 7, 8, 9, 10, 11, 12, 13, 14, 15, 16, 17, or 18;
the sum of q, r, and s is 10, 11, 12, 13, 14, 15, 16, 17, 18, 19, or 20;
t is 1, 2, 3, 4, 5, 6, 7, 8, 9, 10, 11, 12, 13, 14, 15, 16, or 17;
u is 1, 2, 3, 4, 5, 6, 7, 8, 9, 10, 11, 12, 13, 14, 15, 16, or 17;
v is 1, 2, 3, 4, 5, 6, 7, 8, 9, 10, 11, 12, 13, 14, 15, 16, or 17;
w is 0, 1, 2, 3, 4, 5, 6, 7, 8, 9, 10, 11, 12, 13, 14, 15, or 16;
the sum of t, u, v, and w is 9, 10, 11, 12, 13, 14, 15, 16, 17, 18, or 19;
a is 1, 2, 3, 4, 5, 6, 7, 8, 9, 10, 11, 12, 13, 14, or 15;
b is 1, 2, 3, 4, 5, 6, 7, 8, 9, 10, 11, 12, 13, 14, or 15;
c is 1, 2, 3, 4, 5, 6, 7, 8, 9, 10, 11, 12, 13, 14, or 15;
d is 1, 2, 3, 4, 5, 6, 7, 8, 9, 10, 11, 12, 13, 14, or 15;
e is 0, 1, 2, 3, 4, 5, 6, 7, 8, 9, 10, 11, 12, 13, or 14;
the sum of a, b, c, d, and e is 8, 9, 10, 11, 12, 13, 14, 15, 16, 17, or 18;
f is 1, 2, 3, 4, 5, 6, 7, 8, 9, 10, 11, 12, or 13;
g is 1, 2, 3, 4, 5, 6, 7, 8, 9, 10, 11, 12, or 13;
his 1, 2, 3, 4, 5, 6, 7, 8, 9, 10, 11, 12, or 13;
i is 1, 2, 3, 4, 5, 6, 7, 8, 9, 10, 11, 12, or 13;
y is 1, 2, 3, 4, 5, 6, 7, 8, 9, 10, 11, 12, or 13;
z is 0, 1, 2, 3, 4, 5, 6, 7, 8, 9, 10, 11, or 12;
the sum of f, g, h, i, y, and z is 7, 8, 9, 10, 11, 12, 13, 14, 15, 16, or 17; and X¹, X², X³, X⁴, and X⁵ are independently —NH—, —O—, —C(O)NH—, —NHC(O)—, or —NHC(O)—NH—;
wherein

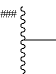

represents a covalent bond to the C(O) group of Formula (L-d), and

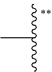

represents a covalent bond to the methylene group of Formula (I).

In another embodiment, L^{1d} of Formula (L-d) is

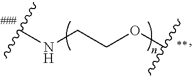

wherein n is 4, 5, 6, 7, 8, 9, or 10;
wherein

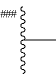

represents a covalent bond to the C(O) group of Formula (L-d), and

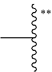

represents a covalent bond to the methylene group of Formula (I).

In another embodiment, L is a divalent linker of Formula (L-d) selected from the group consisting of:

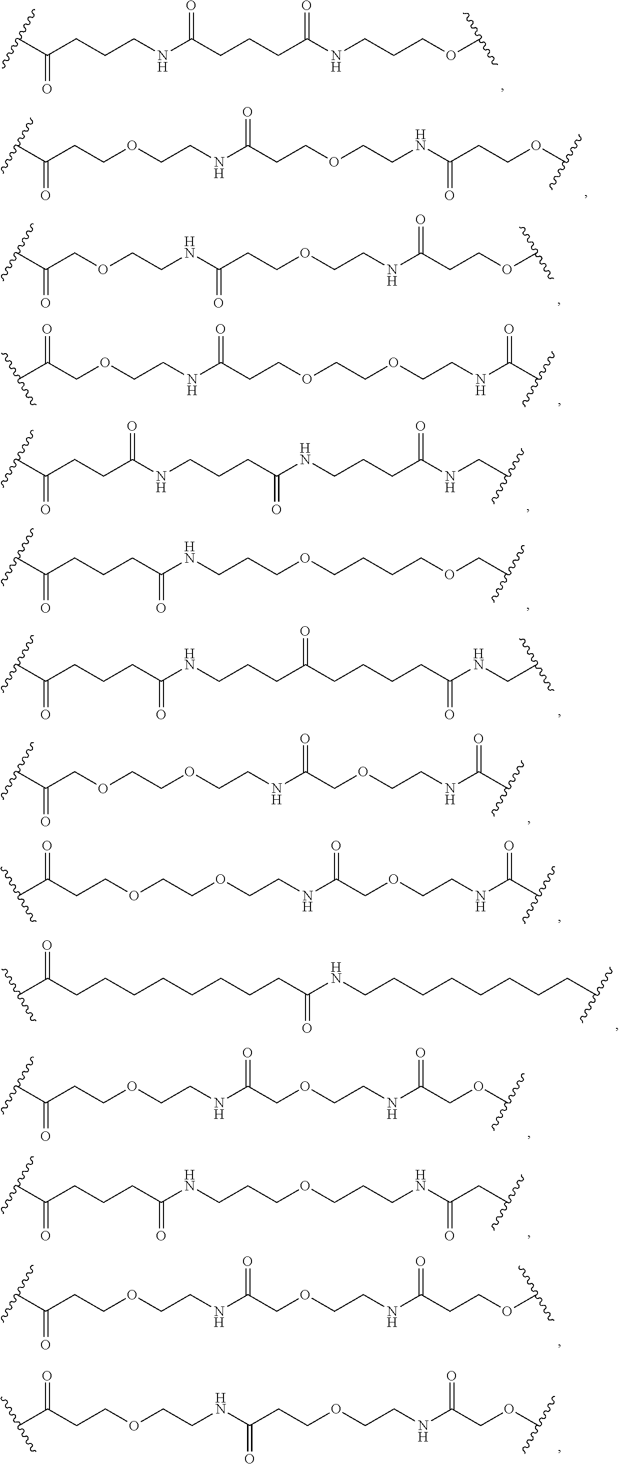

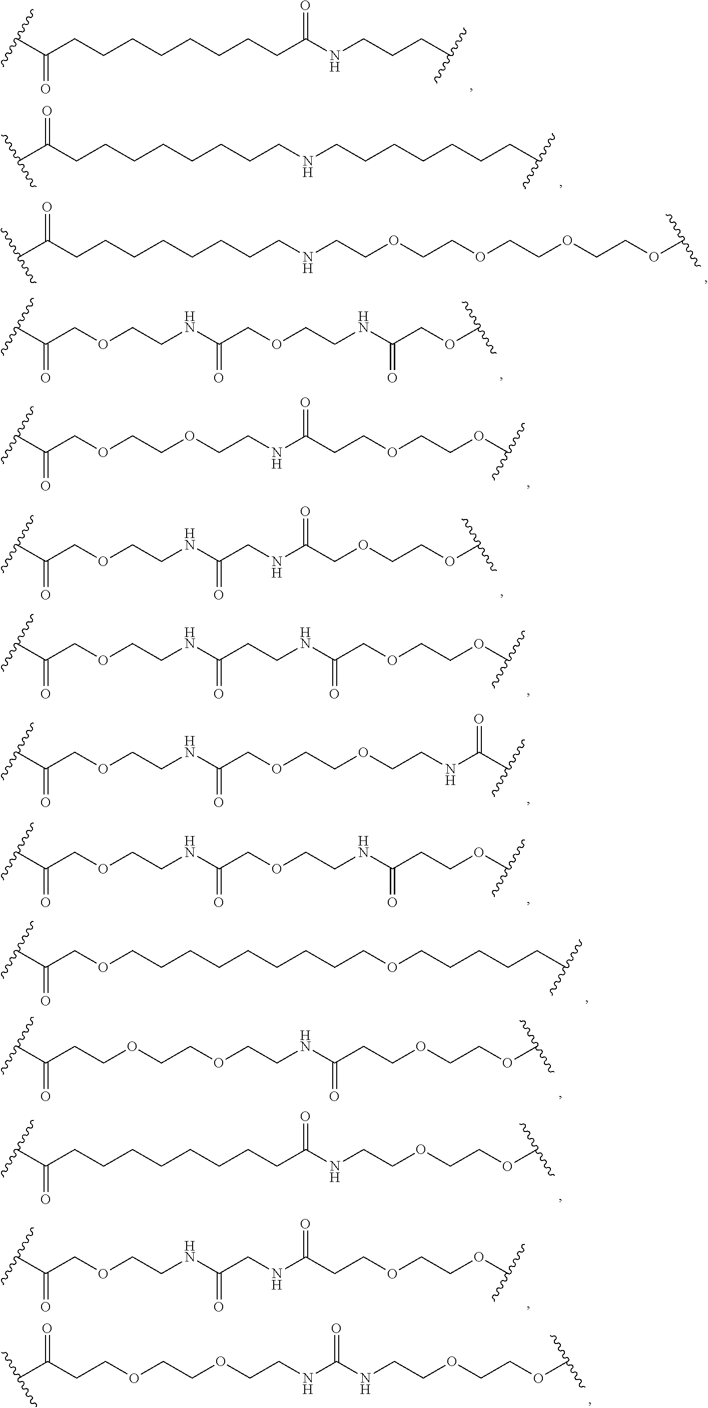

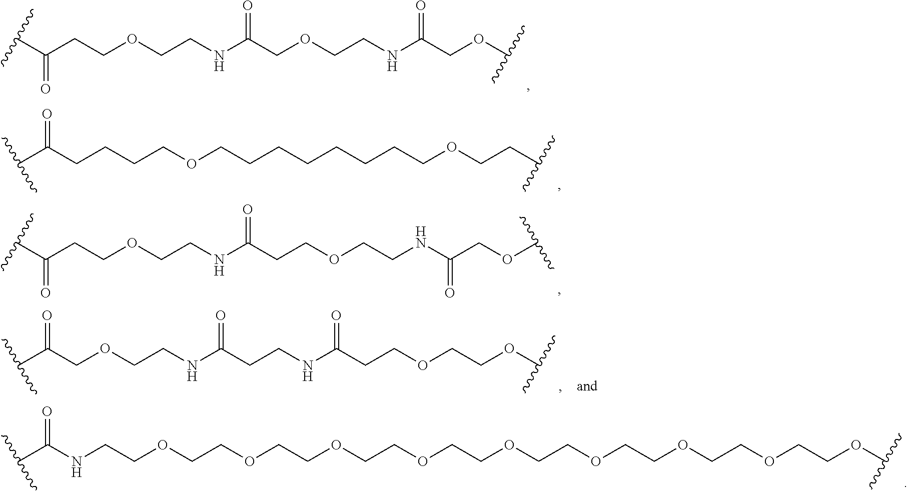

In another embodiment, L is a divalent linker of Formula (L-e):

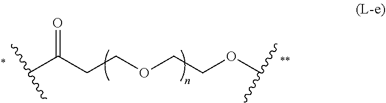

wherein:
n is an integer of 3 to 50;
wherein

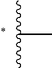

represents a covalent bond to the L′ group of Formula (I), or when L′ is a bond, a covalent bond to the Y group of Formula (I), or when both L′ and Y are a bond, a covalent bond to the T group of Formula (I), and

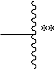

represents a covalent bond to the methylene group of Formula (I).

In another embodiment, n of Formula (L-e) is 3 to 25, 3 to 10, 3 to 8, 3 to 7, 3 to 5, or 3 to 4. In another embodiment, n of Formula (L-e) is 5 to 22, 7 to 15, or 9 to 13. In another embodiment, n of Formula (L-e) is 3, 4, 5, 7, 8, 11, 22, or 50.

In another embodiment, L is a divalent linker of Formula (L-f):

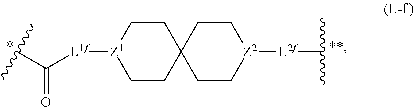

or a stereoisomer thereof,
wherein:
L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—;
L^{2f} is a bond, —NHC(O)—, —C(O)NH—, or a C₁₋₆ linear alkylene, wherein 0, 1, or 2 methylene units are replaced with —O—; and
each of Z¹ and Z² is independently N or CH;
wherein

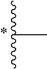

represents a covalent bond to the L′ group of Formula (I), or when L′ is a bond, a covalent bond to the Y group of Formula (I), or when both L′ and Y are a bond, a covalent bond to the T group of Formula (I), and

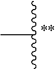

represents a covalent bond to the methylene group of Formula (I).

In another embodiment, L^{1f} of Formula (L-f) is selected from

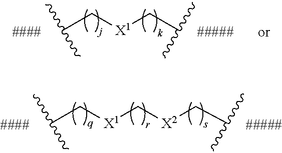

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2;
the sum of q, r, and s is 2, 3, or 4; and
X¹ and X² are independently —O—, —NH—, or —C(O)—; or —(C₃₋₆ cycloalkylene)-NHC(O)—;
wherein

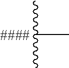

represents a covalent bond to the C(O) group of Formula (L-f), and

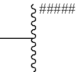

represents a covalent bond to the ring of Formula (L-f).

In another embodiment, L^{2f} of Formula (L-f) is selected from

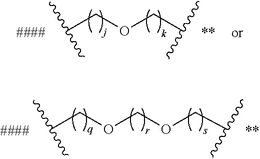

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2; and
the sum of q, r, and s is 2, 3, or 4;
wherein

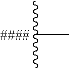

represents a covalent bond to the ring of Formula (L-f), and

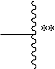

represents a covalent bond to the methylene group of Formula (I).

In another embodiment, L is a divalent linker of Formula (L-f) selected from the group consisting of:

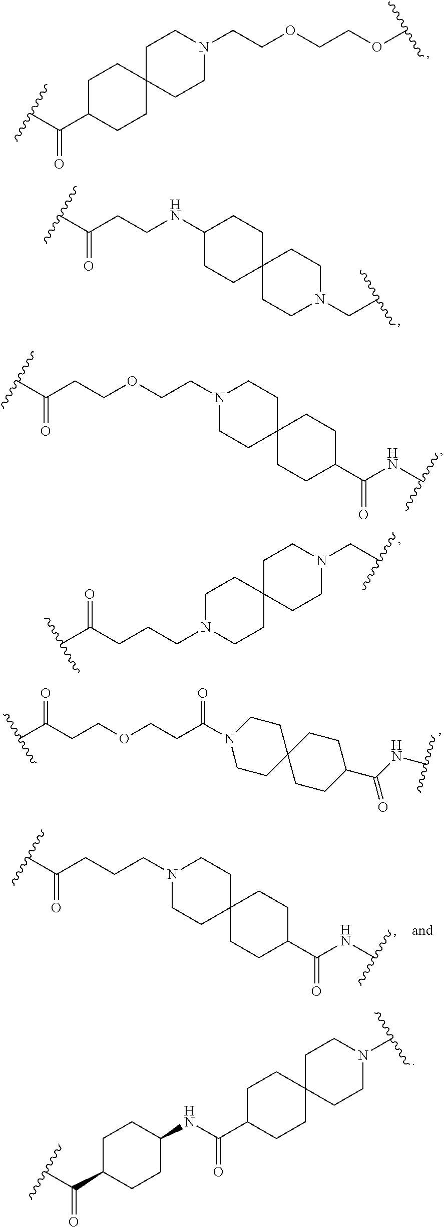

In another embodiment, L is a divalent linker of Formula (L-g):

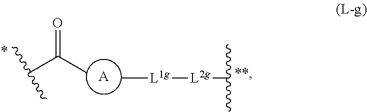

wherein:
Ring A is a 5 to 6 membered heteroarylene having 1 or 2 nitrogen ring atoms;
L^{1g} is a bond, —CH₂—, —NH—, or —O—; and
L^{2g} is

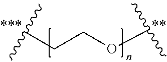

wherein n is 1, 2, 3, 4, or 5, and

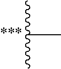

represents a covalent bond to L^{1g};
wherein

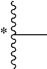

represents a covalent bond to the L′ group of Formula (I), or when L′ is a bond, a covalent bond to the Y group of Formula (I), or when both L′ and Y are a bond, a covalent bond to the T group of Formula (I), and

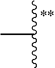

represents a covalent bond to the methylene group of Formula (I).

In another embodiment, L is a divalent linker of Formula (L-g-i):

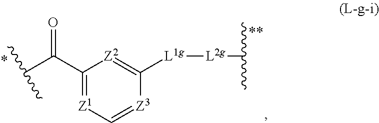

wherein:
L^{1g} is a bond, —CH₂—, —NH—, or —O—;
L^{2g} is

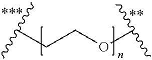

wherein n is 1, 2, 3, 4, or 5, and

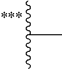

represents a covalent bond to L^{1g};
Z¹, Z², and Z³ are each independently selected from N or CH, provided that one or two of Z¹, Z², and Z³ is N;
wherein

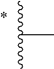

represents a covalent bond to the L′ group of Formula (I), or when L′ is a bond, a covalent bond to the Y group of Formula (I), or when both L′ and Y are a bond, a covalent bond to the T group of Formula (I), and

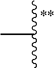

represents a covalent bond to the methylene group of Formula (I).

In another embodiment, L is a divalent linker of Formula (L-g) selected from the group consisting of:

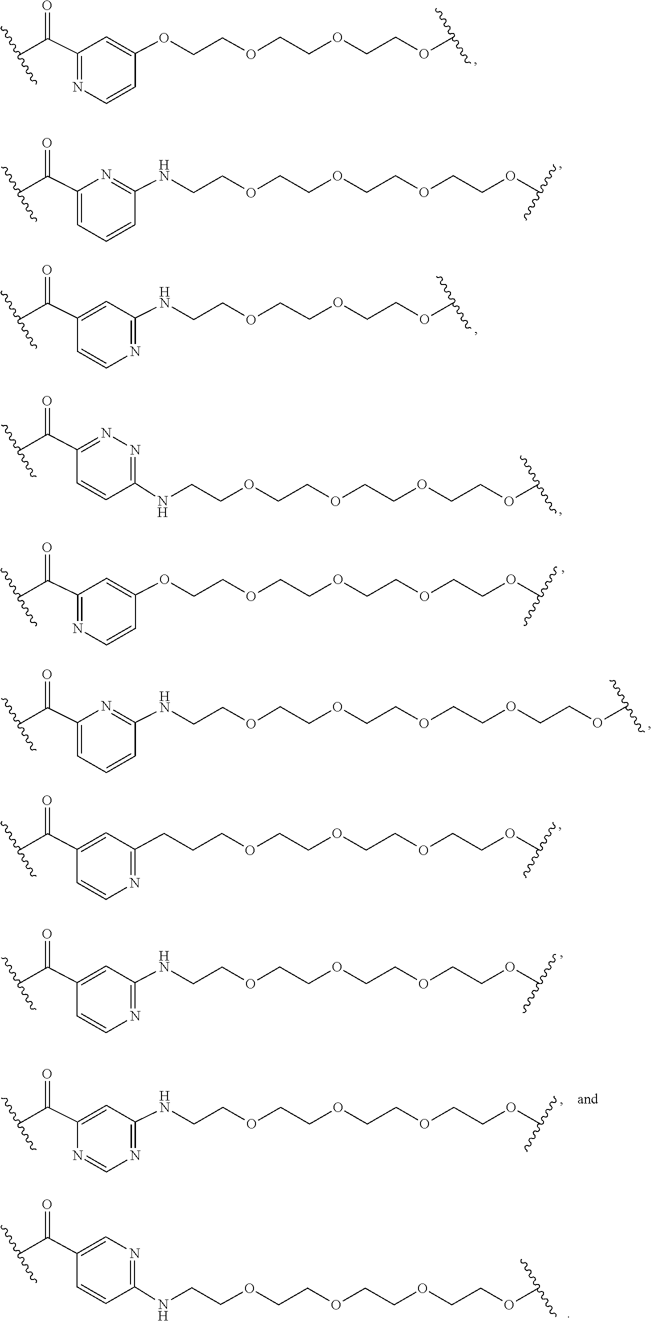

In another embodiment, L is a divalent linker of Formula (L-h):

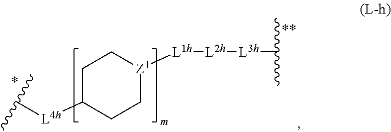

or a stereoisomer thereof,
wherein:
each Z¹ is independently N or CH;
L^{1h} is a bond, —C(O)—, —C(O)—NH—, or —NHC(O)—;
L^{2h} is C₂₋₁₀ linear alkylene or

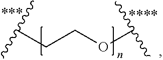

wherein n is 1, 2, 3, or 4, and

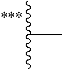

represents a covalent bond to L^{1h} and

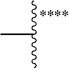

represents a covalent bond to L^{3h};
L^{3h} is a bond, —C(O)CH₂—, —O—(C₃₋₆ cycloalkylene)-O—, or —C(O)NH(CH₂)₃OCH₂—;
L^{4h} is a bond, —C(O)—, —CH₂C(O)—, or —C(O)CH₂—; and
m is 1, 2, or 3;
wherein

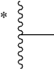

represents a covalent bond to the L′ group of Formula (I), or when L′ is a bond, a covalent bond to the Y group of Formula (I), or when both L′ and Y are a bond, a covalent bond to the T group of Formula (I), and

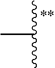

represents a covalent bond to the methylene group of Formula (I).

In another embodiment, L is a divalent linker of Formula (L-h) selected from the group consisting of:

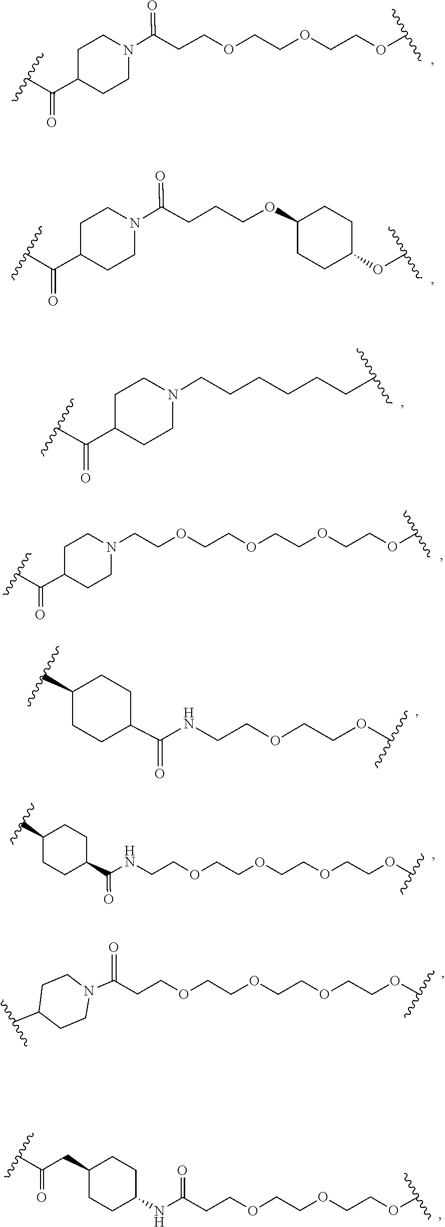

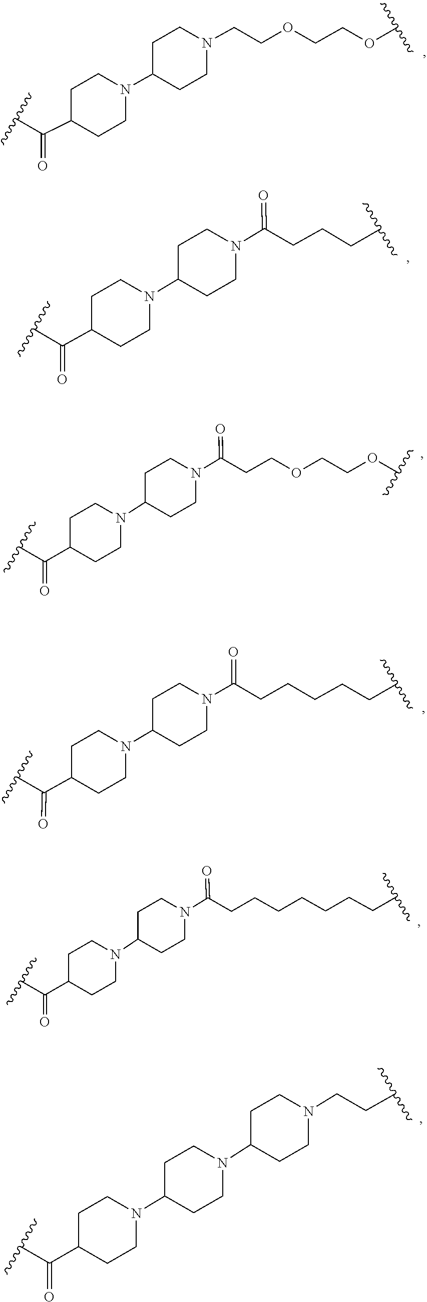 [SMILES: *OCCOCCN1CCC(N2CCC(C(*)=O)CC2)CC1 ; *CCCC(=O)N1CCC(N2CCC(C(*)=O)CC2)CC1 ; *OCCOCCC(=O)N1CCC(N2CCC(C(*)=O)CC2)CC1 ; *CCCCCC(=O)N1CCC(N2CCC(C(*)=O)CC2)CC1 ; *CCCCCCCC(=O)N1CCC(N2CCC(C(*)=O)CC2)CC1 ; *CCN1CCC(N2CCC(N3CCC(C(*)=O)CC3)CC2)CC1]

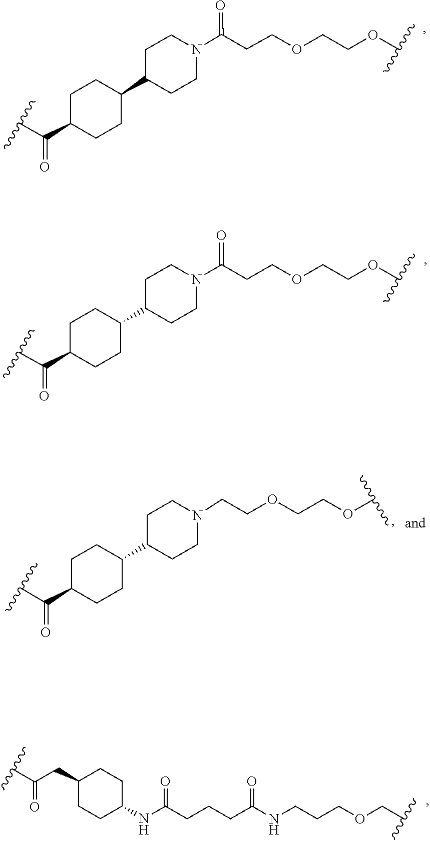 [SMILES: *OCCOCCC(=O)N1CCC([C@H]2CC[C@@H](C(*)=O)CC2)CC1 ; *OCCOCCC(=O)N1CCC([C@H]2CC[C@H](C(*)=O)CC2)CC1 ; *OCCOCCN1CCC([C@H]2CC[C@H](C(*)=O)CC2)CC1 ; *COCCCNC(=O)CCCC(=O)N[C@H]1CC[C@H](CC(*)=O)CC1]

In another embodiment, L is a divalent linker of Formula (L-i):

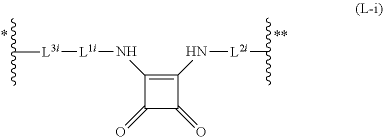

wherein:
L¹ⁱ is a bond, C₁₋₁₂ linear alkylene, or

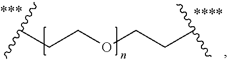

wherein n is 1, 2, 3, 4, or 5, and

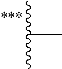

represents a covalent bond to L³ⁱ and

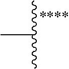

represents a covalent bond to NH;
L²ⁱ is a bond, C₁₋₁₂ linear alkylene, or

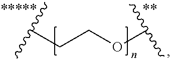

wherein n is 1, 2, 3, 4, or 5, and

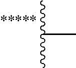

represents a covalent bond to HN; and
L³ⁱ is a bond or —C(O)—;
wherein

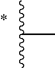

represents a covalent bond to the L′ group of Formula (I), or when L′ is a bond, a covalent bond to the Y group of Formula (I), or when both L′ and Y are a bond, a covalent bond to the T group of Formula (I), and

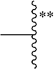

represents a covalent bond to the methylene group of Formula (I).

In another embodiment, L is a divalent linker of Formula (L-i) selected from the group consisting of:

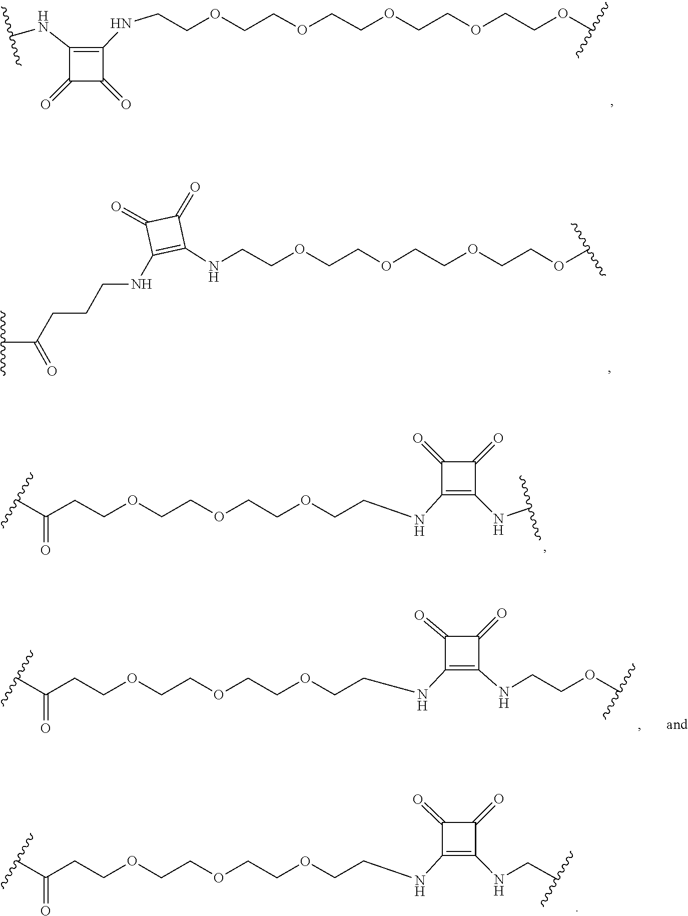 [SMILES: *Nc1c(NCCOCCOCCOCCOCCO*)c(=O)c1=O ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

In another embodiment, L is a divalent linker of Formula (L-j):

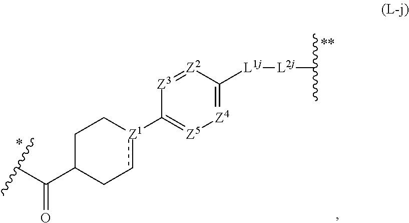

or a stereoisomer thereof,
wherein:
Z¹ is C, CH, or N;
each of Z², Z³, Z⁴ and Z⁵ is independently CH or N, provided that no more than two of Z², Z³, Z⁴ and Z⁵ are N;
L^{1j} is —NH—, —C(O)NH—, —NHC(O)—, or —O—;
L^{2j} is C₁₋₆ linear alkylene or

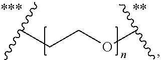

wherein n is 1 or 2, and

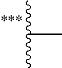

represents a covalent bond to L^{1j}; and
[symbol] represents a single bond or a double bond;
wherein

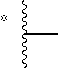

represents a covalent bond to the L′ group of Formula (I), or when L′ is a bond, a covalent bond to the Y group of Formula (I), or when both L′ and Y are a bond, a covalent bond to the T group of Formula (I), and

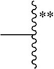

represents a covalent bond to the methylene group of Formula (I).

In another embodiment, L is a divalent linker of Formula (L-j) selected from the group consisting of:

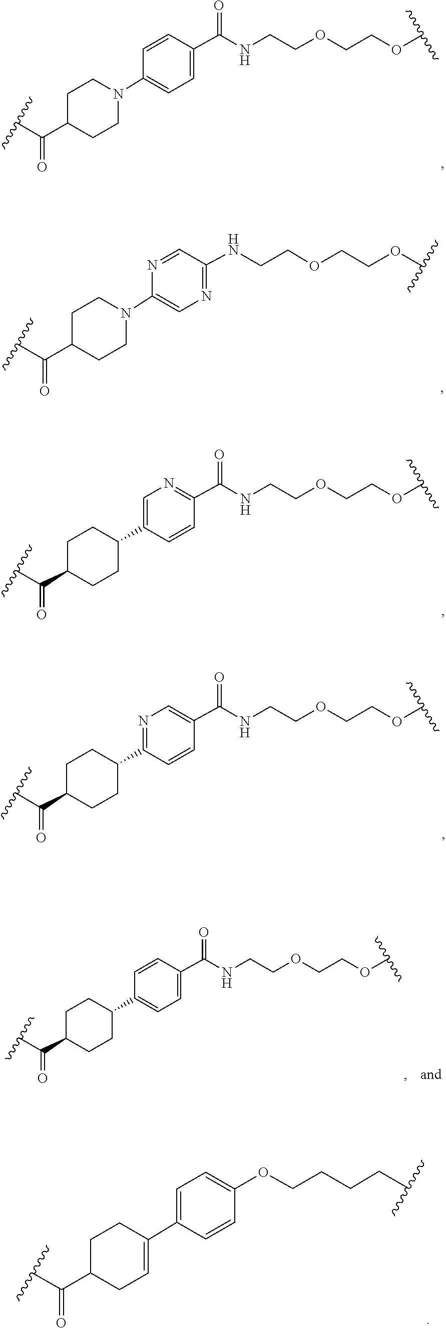 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

In another embodiment, L is a divalent linker of Formula (L-k):

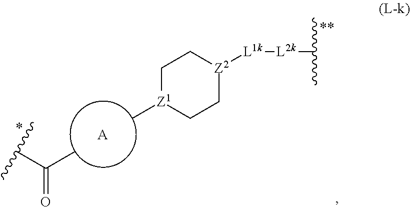

or a stereoisomer thereof,
wherein:
Ring A is phenyl or a 5 or 6 membered heteroarylene having 1 or 2 nitrogen ring atoms;
each of Z¹ and Z² is independently CH or N;
L^{1k} is a bond, —C(O)—, —C(O)NH— or —NHC(O)—; and
L^{2k} is a C₃₋₈ straight chain alkylene or

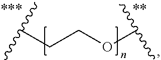

wherein n is 1, 2, or 3, and

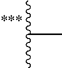

represents a covalent bond to L^{1k};
wherein

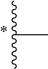

represents a covalent bond to the L′ group of Formula (I), or when L′ is a bond, a covalent bond to the Y group of Formula (I), or when both L′ and Y are a bond, a covalent bond to the T group of Formula (I), and

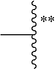

represents a covalent bond to the methylene group of Formula (I).

In another embodiment, L is a divalent linker of Formula (L-k) selected from the group consisting of:

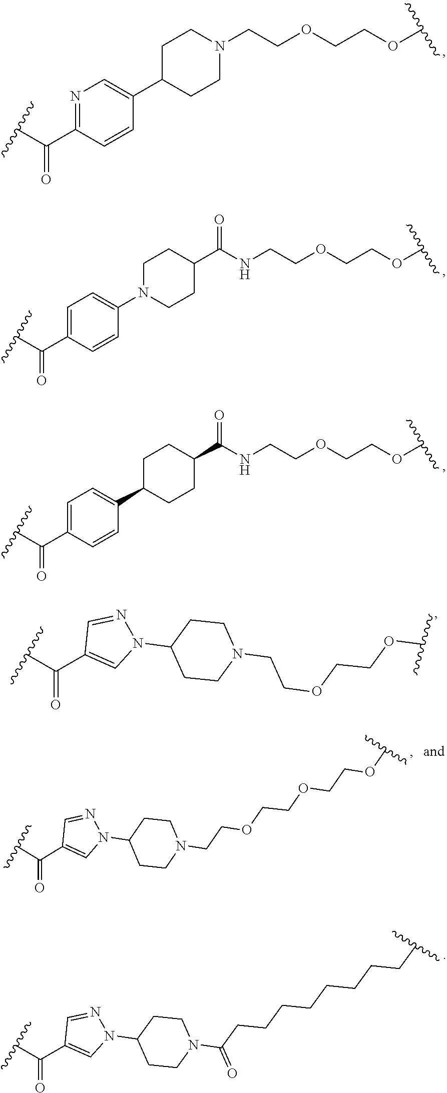 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

In another embodiment, L is a divalent linker of Formula (L-m):

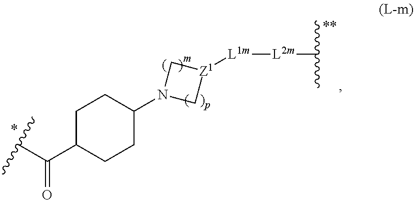

or a stereoisomer thereof,
wherein:
Z¹ is CH or N;
m is 1 or 2;
p is 1 or 2;
0, 1, or 2 hydrogen atoms of

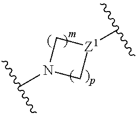

are replaced with F;
L^{1m} is a bond, —C(O)—, —C(O)NH—, —NHC(O)—, —S(O)₂NH— or —NHS(O)₂—; and
L^{2m} is C₃₋₆ linear alkylene, C₃₋₆ cycloalkylene, or

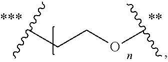

wherein n is 1 or 2, and

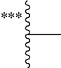

represents a covalent bond to L^{1m};
wherein

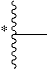

represents a covalent bond to the L′ group of Formula (I), or when L′ is a bond, a covalent bond to the Y group of Formula (I), or when both L′ and Y are a bond, a covalent bond to the T group of Formula (I), and

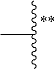

represents a covalent bond to the methylene group of Formula (I).

In another embodiment, L is a divalent linker of Formula (L-m) selected from the group consisting of:

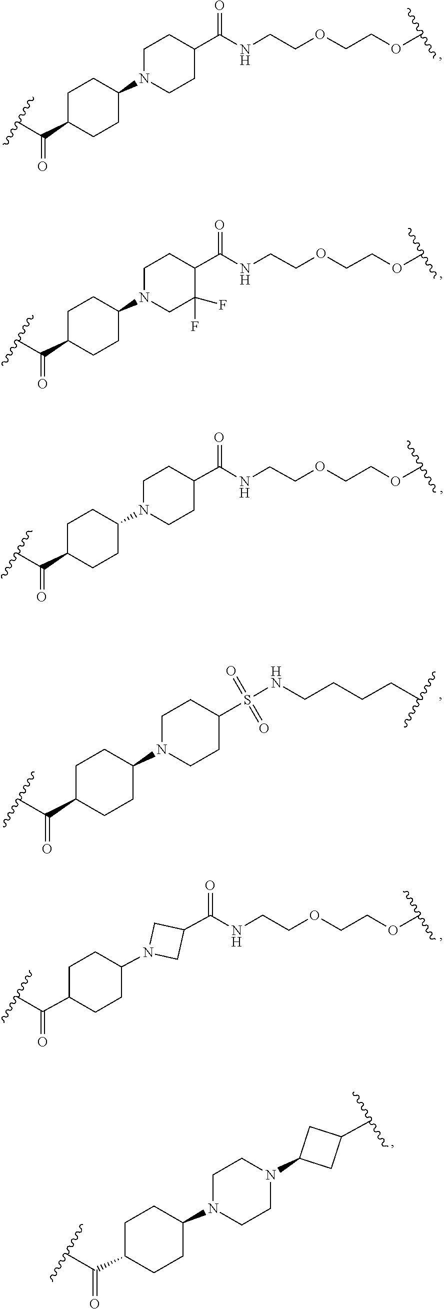

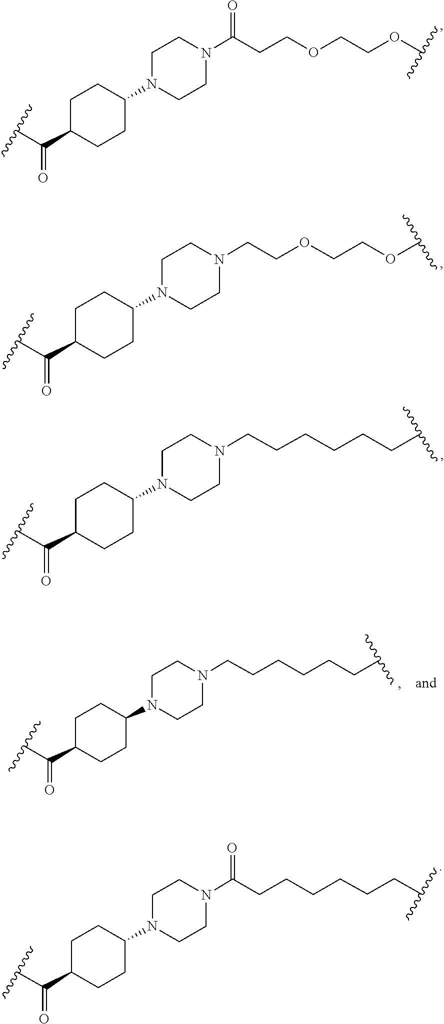 [SMILES: *OCCOCCC(=O)N1CCN([C@H]2CC[C@H](C(*)=O)CC2)CC1 ; *OCCOCCN1CCN([C@H]2CC[C@H](C(*)=O)CC2)CC1 ; *CCCCCCN1CCN([C@H]2CC[C@H](C(*)=O)CC2)CC1 ; *CCCCCCN1CCN([C@H]2CC[C@@H](C(*)=O)CC2)CC1 ; *CCCCCCC(=O)N1CCN([C@H]2CC[C@H](C(*)=O)CC2)CC1]

In another embodiment, L is a divalent linker of Formula (L-n-i):

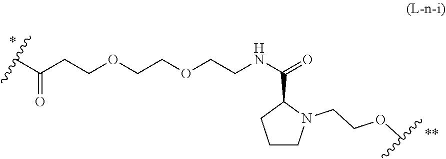 [SMILES: *OCCN1CCC[C@H]1C(=O)NCCOCCOCCC(*)=O]

wherein

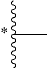

represents a covalent bond to the L′ group of Formula (I), or when L′ is a bond, a covalent bond to the Y group of Formula (I), or when both L′ and Y are a bond, a covalent bond to the T group of Formula (I), and

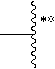

represents a covalent bond to the methylene group of Formula (I).

In another embodiment, L is a divalent linker of Formula (L-n-ii):

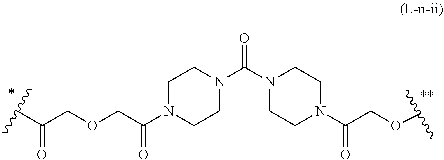 [SMILES: *OCC(=O)N1CCN(C(=O)N2CCN(C(=O)COCC(*)=O)CC2)CC1]

wherein

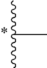

represents a covalent bond to the L′ group of Formula (I), or when L′ is a bond, a covalent bond to the Y group of Formula (I), or when both L′ and Y are a bond, a covalent bond to the T group of Formula (I), and

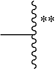

represents a covalent bond to the methylene group of Formula (I).

In another embodiment, L is a divalent linker of Formula (L-n-iii):

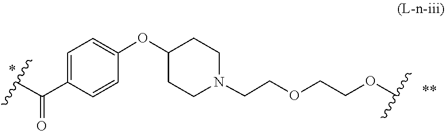 [SMILES: *OCCOCCN1CCC(Oc2ccc(C(*)=O)cc2)CC1]

wherein

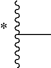

represents a covalent bond to the L′ group of Formula (I), or when L′ is a bond, a covalent bond to the Y group of Formula (I), or when both L′ and Y are a bond, a covalent bond to the T group of Formula (I), and

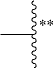

represents a covalent bond to the methylene group of Formula (I).

In another embodiment, L is a divalent linker of Formula (L-n-iv):

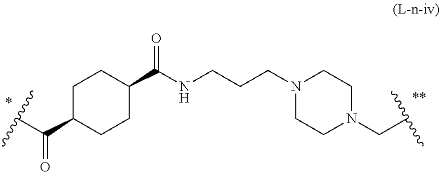 [SMILES: *CN1CCN(CCCNC(=O)[C@H]2CC[C@@H](C(*)=O)CC2)CC1]

wherein

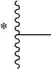

represents a covalent bond to the L′ group of Formula (I), or when L′ is a bond, a covalent bond to the Y group of Formula (I), or when both L′ and Y are a bond, a covalent bond to the T group of Formula (I), and

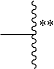

represents a covalent bond to the methylene group of Formula (I).

In another embodiment, L is a divalent linker of Formula (L-p):

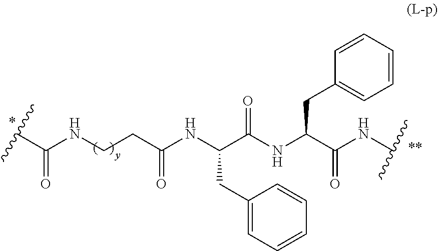

wherein:
y is an integer of 1 to 9;
wherein

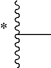

represents a covalent bond to the L′ group of Formula (I), or when L′ is a bond, a covalent bond to the Y group of Formula (I), or when both L′ and Y are a bond, a covalent bond to the T group of Formula (I), and

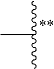

represents a covalent bond to the methylene group of Formula (I).

In another embodiment, y of Formula (L-p) is 2 to 8, 3 to 7, 4 to 7, or 5 to 7. In another embodiment, y of Formula (L-p) is 1, 2, 3, 4, 5, 6, 7, 8, or 9.

In another embodiment, L is a divalent linker of Formula (L-p) having the following structure:

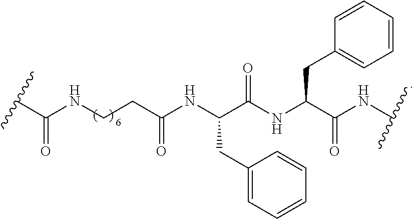

In one embodiment of the disclosure, Y is selected from a bond; —NH—; —(C₁₋₁₂ alkylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene.

In another embodiment, Y is selected from a bond; —NH—; —(C₁₋₆ alkylene)-O—; —(C₂₋₆ alkenylene)-O—; —(C₁₋₆ alkylene)-C(O)—; —(C₂₋₆ alkenylene)-C(O)—; phenylene; piperidinylene; —(C₁₋₆ alkylene)-O-phenylene-; —(C₂₋₆ alkenylene)-O-piperidinylene; —(C₁₋₅ alkylene)-NH—, wherein 0, 1, or 2 methylene units are replaced with —O—; —NH—(C₁₋₅ alkylene)-NH—; —(C₃₋₆ cycloalkylene)-NH—; —(C₃₋₆ cycloalkenylene)-NH—; —C(O)-piperazinylene-; —C(O)NH—(C₁₋₅ alkylene)-NH—; —C(O)NH—(C₂₋₅ alkenylene)-NH—; —C(O)NH—(C₃₋₆ cycloalkylene)-NH—; —C(O)NH—(C₃₋₆ cycloalkenylene)-NH—; or

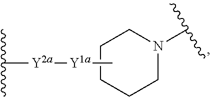

wherein Y^{1a} is a bond, —O—, —NH—, —NHC(O)—, —C(O)NH—, or C₁₋₃ alkylene;
and Y^{2a} is a bond, —O—, —NH—, —NHC(O)—, —C(O)NH—, or C₁₋₃ alkylene. In another embodiment, Y is —NH—.

In another embodiment, Y is selected from the group consisting of:

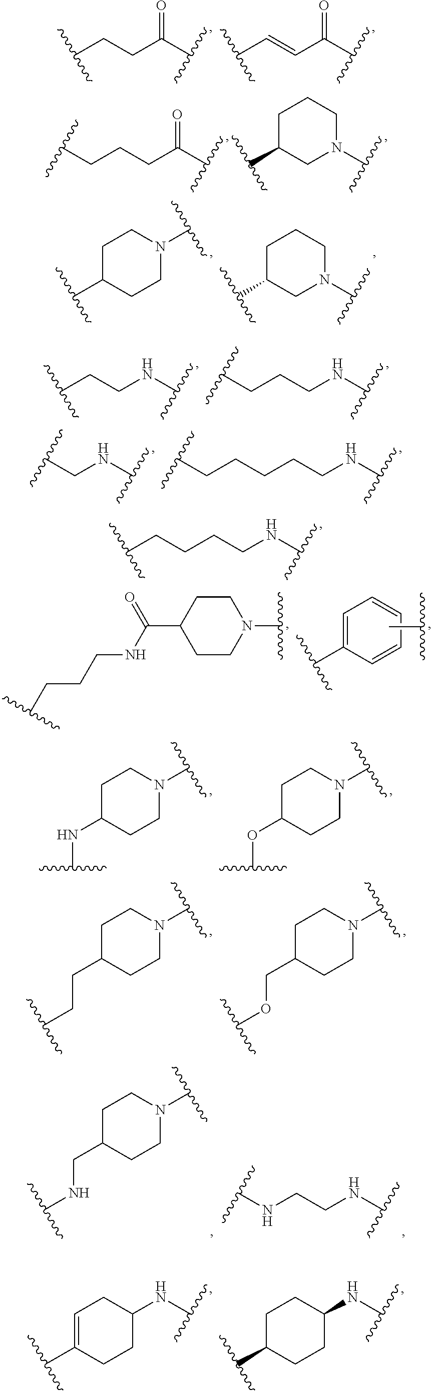

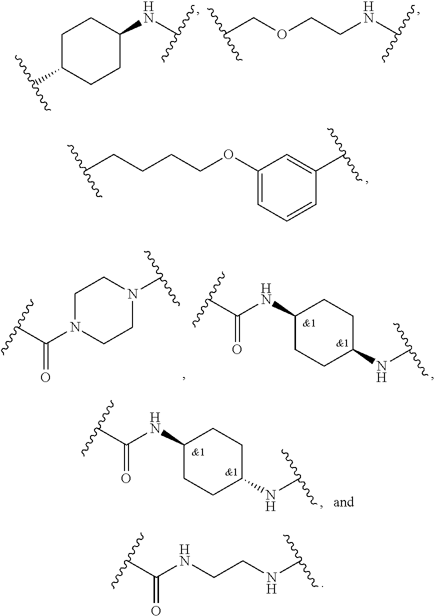

In another embodiment, R¹ is methyl, ethyl, 1-propyl, 2-propyl, 1-butyl, 2-butyl, or t-butyl. In another embodiment, R¹ is methyl. In another embodiment, R¹ is cyclopropyl, cyclobutyl, cyclopentyl, or cyclohexyl.

In another embodiment, y of L′ is 2 to 8, 3 to 7, 4 to 7, or 5 to 7. In another embodiment, y of L′ is 1, 2, 3, 4, 5, 6, 7, 8, or 9.

In another embodiment, w of L′ is 0 to 4, 0 to 3, 0 to 2, or 1 to 2. In another embodiment, w of L′ is 0, 1, 2, 3, 4, or 5.

In another embodiment, L′ is

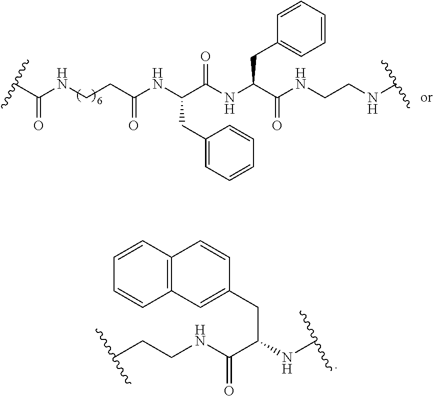

In another embodiment, the compound of Formula (I) is selected from a compound as listed in Table 1:

TABLE 1

| Ex | |
|---|---|
| No. | Structure |
| 1 | 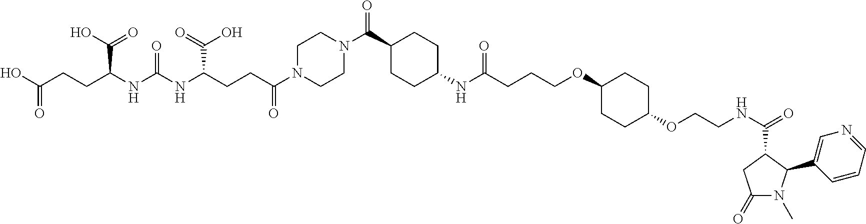 [SMILES: CN1C(=O)C[C@H](C(=O)NCCO[C@H]2CC[C@H](OCCCC(=O)N[C@H]3CC[C@H](C(=O)N4CCN(C(=O)CC[C@H](NC(=O)N[C@@H](CCC(=O)O)C(=O)O)C(=O)O)CC4)CC3)CC2)[C@H]1c1cccnc1] |
| 2 | 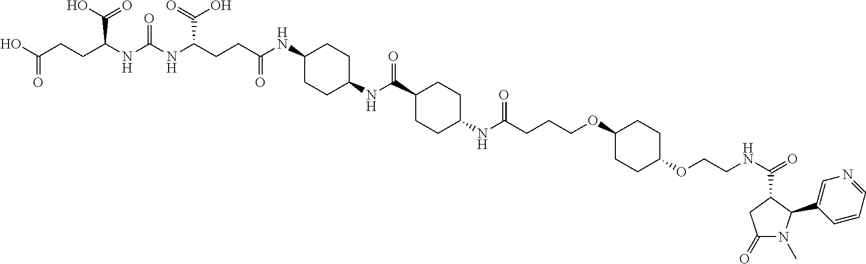 [SMILES: CN1C(=O)C[C@H](C(=O)NCCO[C@H]2CC[C@H](OCCCC(=O)N[C@H]3CC[C@H](C(=O)N[C@H]4CC[C@@H](NC(=O)CC[C@H](NC(=O)N[C@@H](CCC(=O)O)C(=O)O)C(=O)O)CC4)CC3)CC2)[C@H]1c1cccnc1] |
| 3 | 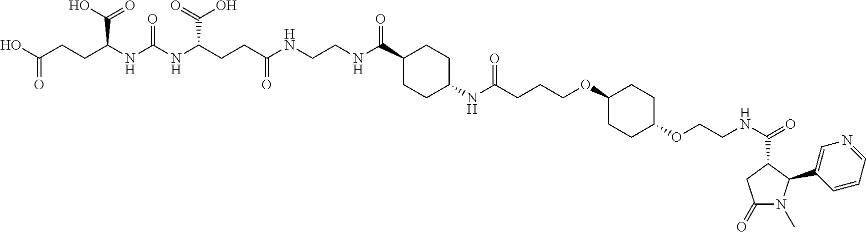 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)N[C@@H](CCC(=O)O)C(=O)O)C(=O)O)CC3)CC2)[C@H]1c1cccnc1] |
| 4 | 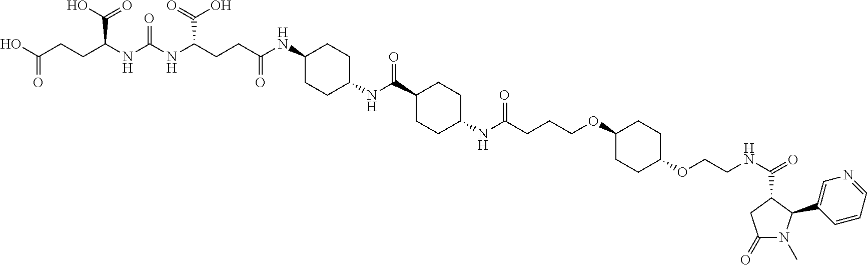 [SMILES: CN1C(=O)C[C@H](C(=O)NCCO[C@H]2CC[C@H](OCCCC(=O)N[C@H]3CC[C@H](C(=O)N[C@H]4CC[C@H](NC(=O)CC[C@H](NC(=O)N[C@@H](CCC(=O)O)C(=O)O)C(=O)O)CC4)CC3)CC2)[C@H]1c1cccnc1] |
| 5 | 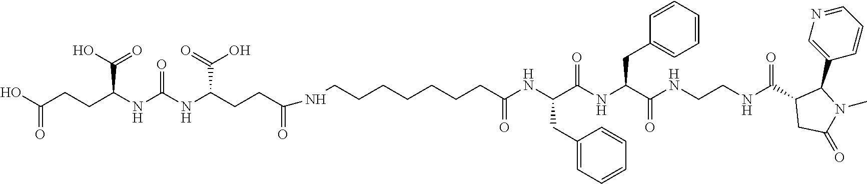 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| 6 | 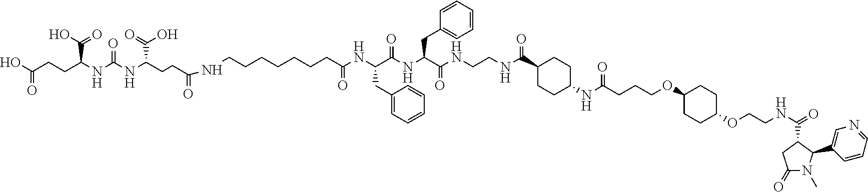 [SMILES: CN1C(=O)C[C@H](C(=O)NCCO[C@H]2CC[C@H](OCCCC(=O)N[C@H]3CC[C@H](C(=O)NCCNC(=O)[C@H](Cc4ccccc4)NC(=O)[C@H](Cc4ccccc4)NC(=O)CCCCCCCNC(=O)CC[C@H](NC(=O)N[C@@H](CCC(=O)O)C(=O)O)C(=O)O)CC3)CC2)[C@H]1c1cccnc1] |
| 7 | 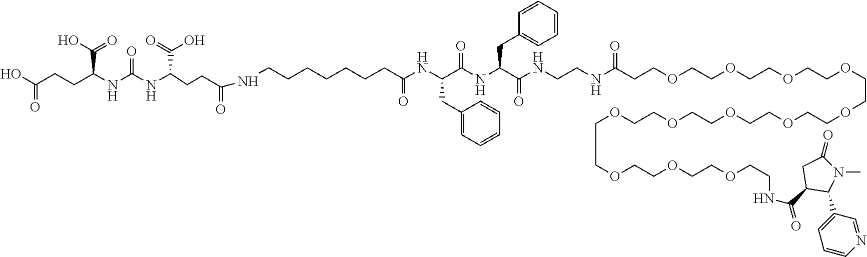 [SMILES: CN1C(=O)C[C@H](C(=O)NCCOCCOCCOCCOCCOCCOCCOCCOCCOCCOCCOCCOCCC(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| 8 | 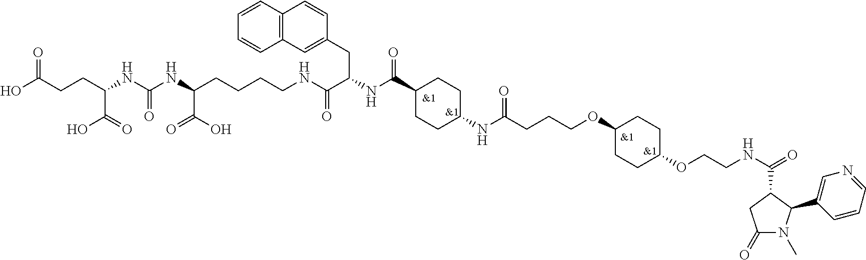 [SMILES: CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1] |
| 9 | 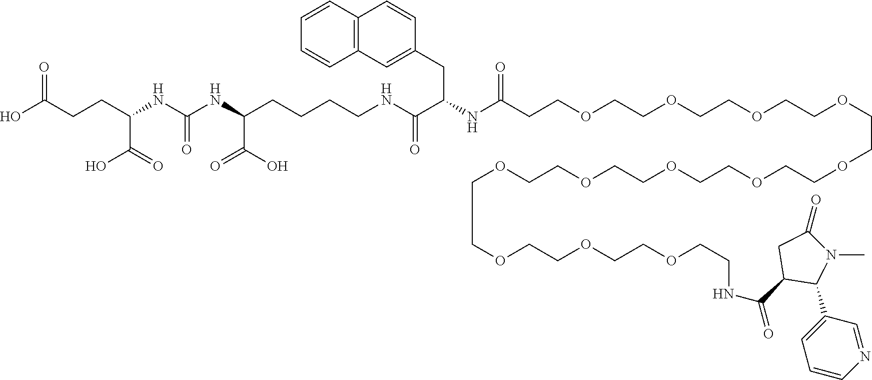 [SMILES: CN1C(=O)C[C@H](C(=O)NCCOCCOCCOCCOCCOCCOCCOCCOCCOCCOCCOCCOCCC(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1] |

In one aspect, the present disclosure provides a compound of Formula (II):

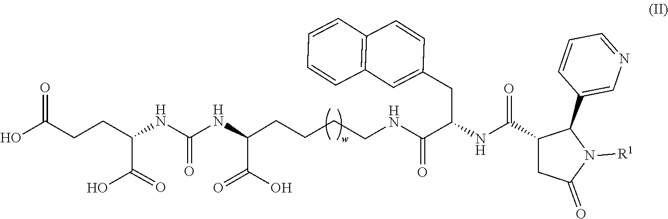

or a pharmaceutically acceptable salt thereof,
wherein:
R¹ is C₁₋₄ alkyl or C₃₋₆ cycloalkyl; and
w is an integer of 0 to 5

In another embodiment, R¹ is methyl, ethyl, 1-propyl, 2-propyl, 1-butyl, 2-butyl, or t-butyl. In another embodiment, R¹ is methyl. In another embodiment, R¹ is cyclopropyl, cyclobutyl, cyclopentyl, or cyclohexyl.

In another embodiment, w of Formula (II) is 0 to 4, 0 to 3, 0 to 2, or 1 to 2. In another embodiment, w of Formula (II) is 0, 1, 2, 3, 4, or 5.

In another embodiment, the compound of Formula (II) is:

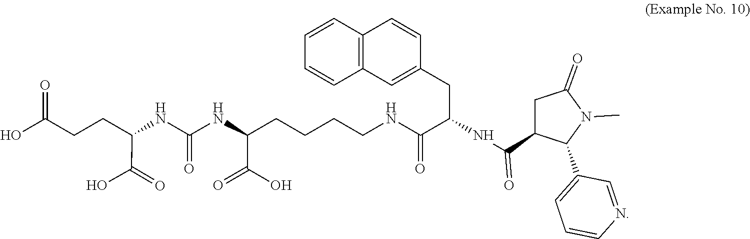 [SMILES: CN1C(=O)C[C@H](C(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1]

### Definitions

As used herein and in the claims, the singular forms “a” and “the” include plural reference unless the context clearly dictates otherwise.

As used herein and in the claims, the term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “pathogenic cells” includes a cell subset that causes or is capable of causing disease. Examples of pathogenic cells include, but are not limited to, cancer or tumor cells, or endothelial cells.

The term “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

The terms “effective amount” and “therapeutically effective amount” refer to an amount of a compound, or antibody, or antigen-binding portion thereof, according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The term “alkyl” represents a saturated, linear or branched hydrocarbon moiety having the specified number of carbon atoms. The term “C₁₋₃ alkyl” refers to an unsubstituted alkyl moiety containing 1, 2 or 3 carbon atoms; exemplary alkyls include methyl, ethyl and propyl.

The term “alkylene” represents a saturated, linear or branched hydrocarbon moiety having the specified number of carbon atoms, with two points of attachment. The two points of attachment can be from the same or different carbon atoms. The term “C₁₋₃ alkylene” refers to an unsubstituted alkyl moiety containing 1, 2 or 3 carbon atoms with two points of attachment; exemplary C₁₋₃ alkylene groups include methylene, ethylene and propylene.

The term “alkenyl” represents an unsaturated, linear or branched hydrocarbon moiety having the specified number of carbon atoms. The term “C₂₋₆ alkenyl” refers to an unsubstituted alkenyl moiety containing 2, 3, 4, 5, or 6 carbon atoms; exemplary alkenyls include propenyl, butenyl, pentenyl and hexenyl.

The term “alkenylene” represents an unsaturated, linear or branched hydrocarbon moiety having the specified number of carbon atoms, with two points of attachment. The two points of attachment can be from the same or different carbon atoms. The term “C₂₋₆ alkenylene” refers to an unsubstituted alkenyl moiety containing 2, 3, 4, 5, or 6 carbon atoms with two points of attachment; exemplary C₂₋₆ alkenylene groups include propenylene, butenylene, pentenylene and hexenylene.

The term “cycloalkyl” represents a saturated cyclic hydrocarbon moiety having the specified number of carbon atoms. The term “C₃₋₆ cycloalkyl” refers to an unsubstituted cycloalkyl moiety containing 3, 4, 5 or 6 carbon atoms; exemplary cycloalkyls include cyclopropyl, cyclobutyl, cyclopentyl, and cyclohexyl.

The term “cycloalkylene” represents a saturated cyclic hydrocarbon moiety having the specified number of carbon atoms, with two points of attachment. The two points of attachment can be from the same or different carbon atoms. The term “C₄₋₆ cycloalkylene” refers to an unsubstituted cycloalkylene moiety containing 4, 5, or 6 carbon atoms with two points of attachment. Exemplary cycloalkylene groups include cyclobutane-1,3-diyl, cyclopentane-1,3-diyl, cyclohexane-1,3-diyl, or cyclohexane-1,4-diyl.

The term “cycloalkenylene” represents an unsaturated cyclic hydrocarbon moiety having the specified number of carbon atoms, with two points of attachment. The two points of attachment can be from the same or different carbon atoms. The term “C₃₋₆ cycloalkenylene” refers to an unsubstituted cycloalkenylene moiety containing 3, 4, 5, or 6 carbon atoms with two points of attachment.

The term “heterocycloalkylene” refers to a saturated cyclic hydrocarbon moiety containing 1 or 2 heteroatoms independently selected from oxygen, sulphur or nitrogen atoms, with two points of attachment. The two points of attachment can be from the same or different carbon atoms. The term “3- to 6-membered heterocycloalkylene” refers to a 3- to 6-membered saturated cyclic moiety containing 2, 3, 4 or 5 carbon atoms in addition to 1 or 2 oxygen, sulphur or nitrogen atoms, with two points of attachment. Suitably, the 3- to 6-membered heterocycloalkylene group contains 1 oxygen or nitrogen atom. Suitably such group contains 3 carbon atoms and 1 oxygen or nitrogen atom, such as azetidindiyl or oxetandiyl. Suitably such group contains 4 or 5 carbon atoms and 1 oxygen or nitrogen atom, such as tetrahydrofurandiyl, tetrahydropyrandiyl, pyrrolidindiyl or piperidindiyl.

The term “bridged bicyclic cycloalkylene” refers to a saturated bicyclic hydrocarbon moiety having at least one bridge, with two points of attachment. A “bridge” is an unbranched chain of atoms or an atom or a valence bond connecting two bridgeheads, where a “bridgehead” is any skeletal atom of the ring system which is bonded to three or more skeletal atoms (excluding hydrogen). The two points of attachment can be from the same or different carbon atoms. The term “C₇₋₉ bridged bicyclic cycloalkylene” refers to an unsubstituted bridged bicyclic cycloalkylene moiety containing 7, 8, or 9 carbon atoms with two points of attachment.

The term “arylene” refers to a monocyclic or bicyclic ring system wherein at least one ring in the system is aromatic, with two points of attachment. Exemplary arylene groups include phenylene, biphenylene, naphthylene, and anthracylene.

The term “heteroarylene” refers to a monocyclic or bicyclic ring system wherein at least one ring in the system is aromatic, and having, in addition to carbon atoms, from one to five heteroatoms independently selected from oxygen, sulphur or nitrogen atoms, with two points of attachment. The term “5- to 6-membered heteroarylene” refers to a 5- to 6-membered cyclic aromatic moiety containing 2, 3, 4 or 5 carbon atoms in addition to 1, 2, or 3 heteroatoms independently selected from oxygen, sulphur or nitrogen atoms, with two points of attachment.

The skilled artisan will appreciate that salts, including pharmaceutically acceptable salts, of the compounds according to Formulae (I) and (II) may be prepared. Indeed, in certain embodiments of the invention, salts including pharmaceutically-acceptable salts of the compounds according to Formulae (I) and (II) may be preferred over the respective free or unsalted compound. Accordingly, the invention is further directed to salts, including pharmaceutically-acceptable salts, of the compounds according to Formulae (I) and (II). The invention is further directed to free or unsalted compounds of Formulae (I) and (II).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, trifluoroacetate, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), b/s-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formulae (I) and (II) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formulae (I) or (II), or in any chemical structure illustrated herein, if not specified the structure is intended to encompass all individual stereoisomers and all mixtures thereof. Thus, compounds according to Formulae (I) and (II) containing one or more chiral centers may be used as racemic mixtures, enantiomerically enriched mixtures, or as enantiomerically pure individual stereoisomers.

A mixture of stereoisomers in which the relative configuration of all of the stereocenters is known may be depicted using the symbol “&” together with an index number (e.g., “&1”). For example, a group of two stereogenic centers labeled with the symbol “&1” represents a mixture of two possible stereoisomers in which the two stereogenic centers have a relative configuration as depicted.

Divalent groups are groups having two points of attachment. For all divalent groups, unless otherwise specified, the orientation of the group is implied by the direction in which the formula or structure of the group is written.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range.

Concentrations described herein are determined at ambient temperature and pressure. This may be, for example, the temperature and pressure at room temperature or in a particular portion of a process stream. Preferably, concentrations are determined at a standard state of 25° C. and 1 bar of pressure.

### PSMA Target and PSMA-Binding Moieties

The compounds of Formulae (I) and (II) as disclosed herein are heterobifunctional synthetic agents designed such that one terminus interacts with a cell surface PSMA target, while the other terminus binds a specific antibody. More specifically, the ARM simultaneously binds the cell surface PSMA target as well as the specific antibody. This ternary complex directs immune surveillance to PSMA-expressing tissue/cells and unites the mechanisms of antibody function with the dose-control of small molecules. This mechanism may include antibody dependent cellular cytotoxicity (ADCC), antibody dependent cellular phagocytosis (ADCP), or complement dependant cytotoxicity (CDC), and preferably includes ADCC. The same Fc receptor expressing immune cells that initiate destruction of the ARM/antibody tagged cells also participate in presentation of endogenous antigens for the potential for long term cellular immunity.

The compounds of Formulae (I) and (II) as disclosed herein include a PSMA-binding moiety that is capable of binding PSMA present on the surface of a cell. In one embodiment, the PSMA is expressed on a pathogenic cell.

In a further embodiment, the pathogenic cell is a tumor cell or cancer cell, or endothelial cell associated with tumor neovasculature.

In a further embodiment, the tumor cells or cancer cells are solid tumor cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, renal cell cancer cells, bladder cancer cells, or breast cancer cells. In some embodiments, the tumor cells or cancer cells are mCRPC cells, breast cancer cells, lung cancer cells, or renal cell cancer cells.

In a further embodiment, the pathogenic cell is an endothelial cell associated with tumor neovasculature.

The present disclosure also provides a pharmaceutical composition comprising a compound of Formulae (I) or (II) as disclosed herein, and a pharmaceutically acceptable excipient, carrier, or diluent.

### Anti-Cotinine Antibodies

The present disclosure provides an antibody, or antigen-binding fragment thereof, that binds to a cotinine moiety. As used herein, the term “anti-cotinine antibody or antigen-binding fragment thereof” refers to an antibody, or antigen binding fragment thereof that binds to a cotinine moiety. Cotinine has the following structure:

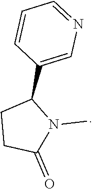

As used herein, the term “cotinine moiety” refers to cotinine or an analog of cotinine. Compounds of Formulae (I) and (II) described herein comprise a cotinine moiety linked via a linker to a PSMA-binding moiety. In one embodiment, the cotinine moiety has the following structure:

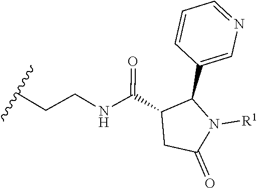

wherein R¹ is C₁₋₄ alkyl or C₃₋₆ cycloalkyl. In another embodiment, R¹ is methyl, ethyl, 1-propyl, 2-propyl, 1-butyl, 2-butyl, or t-butyl. In another embodiment, R¹ is methyl. In another embodiment, R¹ is cyclopropyl, cyclobutyl, cyclopentyl, or cyclohexyl.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)2, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, 23 (9): 1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or λ light chain. The majority of antibodies in the serum belong to the IgG class, there are four isotypes of human IgG (IgG1, IgG2, IgG3 and IgG4), the sequences of which differ mainly in their hinge region.

“CDRs” are defined as the complementarity determining region amino acid sequences of an antibody or antigen binding fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature, 1989, 342:877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 2 below represents one definition using each numbering convention for each CDR or binding unit. It should be noted that some of the CDR definitions may vary depending on the individual publication used.

TABLE 2

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

In a further embodiment, the anti-cotinine antibody is humanized. In a further embodiment, the Fc region of the anti-cotinine antibody is modified to increase ADCC activity, ADCP activity, and/or CDC activity, suitable modifications of which are provided below. In a further embodiment, the Fc region of the anti-cotinine antibody is modified to increase ADCC activity.

Fc engineering methods can be applied to modify the functional or pharmacokinetics properties of an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function. The in vivo half-life of an antibody can be altered by making mutations that affect binding of the Fc to the FcRn (neonatal Fc receptor).

The term “effector function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antibody, or antigen binding fragment thereof, of the present invention can be assessed for ADCC effector function in a Natural Killer cell assay. Examples of such assays can be found in Shields et al., The Journal of Biological Chemistry, 2001, 276:6591-6604; Chappel et al., The Journal of Biological Chemistry, 1993, 268:25124-25131; Lazar et al., PNAS, 2006, 103:4005-4010.

Examples of assays to determine CDC function include those described in J Imm Meth, 1995, 184:29-38.

The effects of mutations on effector functions (e.g., FcRn binding, FcγRs and C1q binding, CDC, ADCML, ADCC, ADCP) can be assessed, e.g., as described in Grevys et al., J Immunol., 2015, 194 (11): 5497-5508; Tam et al., Antibodies, 2017, 6 (3): 12; or Monnet et al., mAbs, 2014, 6 (2): 422-436.

Throughout this specification, amino acid residues in Fc regions, in antibody sequences or full-length antigen binding protein sequences, are numbered according to the EU index numbering convention.

Human IgG1 constant regions containing specific mutations have been shown to enhance binding to Fc receptors. In some cases these mutations have also been shown to enhance effector functions, such as ADCC and CDC, as described below. Antibodies, or antigen binding fragments thereof, of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science, 2014, 343:1260-1293) and E345R/E430G/S440Y results in preformed IgG hexamers (Wang et al., Protein Cell, 2018, 9(1): 63-73).

Enhanced ADCC: Fc engineering can be used to enhance ADCC. For example (with reference to IgG1), F243L/R292P/Y300L/V305I/P396L; S239D/I332E; and S298A/E333A/K334A increase FcγRIIIa binding; S239D/I332E/A330L increases FcγRIIIa binding and decreases FcγRIIb binding; G236A/S239D/I332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., mAbs, 2013, 5 (2): 229-236).

Enhanced ADCP: Fc engineering can be used to enhance ADCP. For example (with reference to IgG1), G236A/S239D/I332E increases FcγRIIa binding and increases FcγRIIIa binding (Richards, J. et al., Mol. Cancer Ther., 2008, 7:2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding Wang et al., Protein Cell, 2018, 9(1): 63-73).

In a further embodiment, an antibody, or antigen binding fragment thereof, of the present invention may comprise a heavy chain constant region with an altered glycosylation profile, such that the antibody, or antigen binding fragment thereof, has an enhanced effector function, e.g., enhanced ADCC, enhanced CDC, or both enhanced ADCC and CDC. Examples of suitable methodologies to produce an antibody, or antigen binding fragment thereof, with an altered glycosylation profile are described in WO 2003/011878, WO 2006/014679 and EP1229125.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al., J Biol Chem., 2002, 277(30): 26733-40 and Monnet et al., mAbs, 2014, 6(2): 422-436).

In one embodiment there is provided an antibody, or antigen binding fragment thereof, comprising a chimeric heavy chain constant region. In an embodiment, the antibody, or antigen binding fragment thereof, comprises an IgG1/IgG3 chimeric heavy chain constant region, such that the antibody, or antigen binding fragment thereof, has an enhanced effector function, for example enhanced ADCC or enhanced CDC, or enhanced ADCC and CDC functions. For example, a chimeric antibody, or antigen binding fragment thereof, of the invention may comprise at least one CH2 domain from IgG3. In one such embodiment, the antibody, or antigen binding fragment thereof, comprises one CH2 domain from IgG3 or both CH2 domains may be from IgG3. In a further embodiment, the chimeric antibody, or antigen binding fragment thereof, comprises an IgG1 CH1 domain, an IgG3 CH2 domain, and an IgG3 CH3 domain. In a further embodiment, the chimeric antibody, or antigen binding fragment thereof, comprises an IgG1 CH1 domain, an IgG3 CH2 domain, and an IgG3 CH3 domain except for position 435 that is histidine.

In a further embodiment, the chimeric antibody, or antigen binding fragment thereof, comprises an IgG1 CH1 domain and at least one CH2 domain from IgG3. In an embodiment, the chimeric antibody, or antigen binding fragment thereof, comprises an IgG1 CH1 domain and the following residues, which correspond to IgG3 residues, in a CH2 domain: 274Q, 276K, 296F, 300F and 339T. In an embodiment, the chimeric antibody, or antigen binding fragment thereof, also comprises 356E, which corresponds to an IgG3 residue, within a CH3 domain. In an embodiment, the antibody, or antigen binding fragment thereof, also comprises one or more of the following residues, which correspond to IgG3 residues within a CH3 domain: 358M, 384S,392N, 397M, 422I, 435R, and 436F.

Also provided is a method of producing an antibody, or antigen binding fragment thereof, according to the invention comprising the steps of:
a) culturing a recombinant host cell comprising an expression vector comprising a nucleic acid sequence encoding a chimeric Fc region having both IgG1 and IgG3 Fc region amino acid residues (e.g. as described above); and
b) recovering the antibody, or antigen binding fragment thereof.

Such methods for the production of antibody, or antigen binding fragment thereof, with chimeric heavy chain constant regions can be performed, for example, using the COMPLEGENT technology system available from BioWa, Inc. (Princeton, NJ) and Kyowa Hakko Kirin Co., Ltd. The COMPLEGENT system comprises a recombinant host cell comprising an expression vector in which a nucleic acid sequence encoding a chimeric Fc region having both IgG1 and IgG3 Fc region amino acid residues is expressed to produce an antibody, or antigen binding fragment thereof, having enhanced CDC activity, i.e. CDC activity is increased relative to an otherwise identical antibody, or antigen binding fragment thereof, lacking such a chimeric Fc region, as described in WO 2007/011041 and US 2007/0148165, each of which are incorporated herein by reference. In an alternative embodiment, CDC activity may be increased by introducing sequence specific mutations into the Fc region of an IgG chain. Those of ordinary skill in the art will also recognize other appropriate systems.

The present invention also provides a method of producing an antibody, or antigen binding fragment thereof, according to the invention comprising the steps of:
a) culturing a recombinant host cell comprising an expression vector comprising a nucleic acid encoding the antibody, or antigen binding fragment thereof, optionally wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO 00/61739 and WO 02/31240, all of which are incorporated herein by reference. Those of ordinary skill in the art will also recognize other appropriate systems.

In one embodiment, the antibody, or antigen binding fragment thereof, is produced in a host cell in which the FUT8 gene has been inactivated. In a further embodiment, the antibody, or antigen binding fragment thereof, is produced in a −/−FUT8 host cell. In a further embodiment, the antibody, or antigen binding fragment thereof, is afucosylated at Asn297 (IgG1).

It will be apparent to those skilled in the art that such modifications may not only be used alone but may be used in combination with each other in order to further enhance effector function.

In one such embodiment, there is provided an antibody, or antigen binding fragment thereof, comprising a heavy chain constant region that comprises a both a mutated and chimeric heavy chain constant region, individually described above. For example, an antibody, or antigen binding fragment thereof, comprising at least one CH2 domain from IgG3 and one CH2 domain from IgG1, and wherein the IgG1 CH2 domain has one or more mutations at positions selected from 239, 332 and 330 (for example the mutations may be selected from S239D, 1332E and A330L), such that the antibody, or antigen binding fragment thereof, has enhanced effector function, e.g. enhanced ADCC or enhanced CDC, or enhanced ADCC and enhanced CDC in comparison to an equivalent antibody, or antigen binding fragment thereof, with an IgG1 heavy chain constant region lacking said mutations. In one embodiment, the IgG1 CH2 domain has the mutations S239D and 1332E. In another embodiment, the IgG1 CH2 domain has the mutations S239D, A330L, and 1332E.

In an alternative embodiment, there is provided an antibody, or antigen binding fragment thereof, comprising both a chimeric heavy chain constant region and an altered glycosylation profile, as individually described above. In an embodiment, the antibody, or antigen binding fragment thereof, comprises an altered glycosylation profile such that the ratio of fucose to mannose is 0.8:3 or less. In one such embodiment, the heavy chain constant region comprises at least one CH2 domain from IgG3 and one CH2 domain from IgG1 and has an altered glycosylation profile such that the ratio of fucose to mannose is 0.8:3 or less, for example wherein the antibody, or antigen binding fragment thereof, is defucosylated. Said antibody, or antigen binding fragment thereof, has an enhanced effector function, e.g. enhanced ADCC or enhanced CDC, or enhanced ADCC and enhanced CDC, in comparison to an equivalent antibody, or antigen binding fragment thereof, with an IgG1 heavy chain constant region lacking said glycosylation profile.

In an alternative embodiment, the antibody, or antigen binding fragment thereof, has at least one IgG3 heavy chain CH2 domain and at least one heavy chain constant domain from IgG1 wherein both IgG CH2 domains are mutated in accordance with the limitations described herein.

In one aspect, there is provided a method of producing an antibody, or antigen binding fragment thereof, according to the invention described herein comprising the steps of:
a) culturing a recombinant host cell containing an expression vector comprising a nucleic acid sequence encoding a chimeric Fc domain having both IgG1 and IgG3 Fc domain amino acid residues (e.g. as described above); and wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the ACCRETAMAB technology system available from BioWa, Inc. (Princeton, NJ) that combines the POTELLIGENT and COMPLEGENT technology systems to produce an antibody, or antigen binding fragment thereof, having both enhanced ADCC and CDC activity relative to an otherwise identical monoclonal antibody that lacks a chimeric Fc domain and that is fucosylated.

In another embodiment, there is provided an antibody, or antigen binding fragment thereof, comprising a mutated and chimeric heavy chain constant region wherein said antibody, or antigen binding fragment thereof, has an altered glycosylation profile such that the antibody, or antigen binding fragment thereof, has enhanced effector function, e.g. enhanced ADCC or enhanced CDC, or both enhanced ADCC and CDC. In one embodiment the mutations are selected from positions 239, 332 and 330, e.g. S239D, 1332E and A330L. In a further embodiment the heavy chain constant region comprises at least one CH2 domain from IgG3 and one CH1 domain from IgG1. In one embodiment the heavy chain constant region has an altered glycosylation profile such that the ratio of fucose to mannose is 0.8:3 or less, e.g. the antibody, or antigen binding fragment thereof, is defucosylated, such that said antibody, or antigen binding fragment thereof, has an enhanced effector function in comparison with an equivalent non-chimeric antibody, or antigen binding fragment thereof, lacking said mutations and lacking said altered glycosylation profile.

In a further embodiment, the anti-cotinine antibody, or antigen binding fragment thereof, comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In a further embodiment, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/I332E or S239D/I332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody, or antigen binding fragment thereof, comprises a heavy chain variable region (VH) having SEQ ID NO: 7, a light chain variable region (VL) having SEQ ID NO: 8. In a further embodiment, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/I332E or S239D/I332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody has a heavy chain comprising SEQ ID NO: 9 and a light chain comprising SEQ ID NO: 10.

The present disclosure also provides a pharmaceutical composition comprising an anti-cotinine antibody, or antigen binding fragment thereof as disclosed herein, and a pharmaceutically acceptable excipient, carrier, or diluent.

The present disclosure also provides a combination comprising the compound of Formulae (I) or (II) as disclosed herein, preferably a compound of Formula (I) as disclosed herein, and an anti-cotinine antibody, or antigen-binding fragment thereof as disclosed herein. The compound of Formulae (I) or (II) and anti-cotinine antibody, or antigen binding fragment thereof can be present in the same composition or in separate compositions. In one embodiment, a combination comprises a pharmaceutical composition comprising the compound of Formulae (I) or (II) as disclosed herein, preferably a compound of Formula (I) as disclosed herein, and an anti-cotinine antibody, or antigen binding fragment thereof as disclosed herein, and a pharmaceutically acceptable carrier, diluent, or excipient. In another embodiment, a combination comprises a first pharmaceutical composition comprising a compound of Formulae (I) or (II) as disclosed herein, preferably a compound of Formula (I) as disclosed herein, and a pharmaceutically acceptable carrier, diluent, or excipient; and a second pharmaceutical composition comprising an anti-cotinine antibody or antigen binding fragment thereof as disclosed herein, and a pharmaceutically acceptable carrier, excipient, or diluent.

### STATEMENT OF USE

The compounds of Formula (I) and pharmaceutically acceptable salts thereof are capable of simultaneously binding a cell surface-expressed PSMA and an anti-cotinine antibody, or antigen binding fragment thereof to form a ternary complex for the treatment and/or prevention of diseases or disorders associated with PSMA-expressing cells.

In one embodiment, the present disclosure provides a method of treating and/or preventing a disease or disorder in a patient in need thereof comprising administering to the patient a therapeutically effective amount of the compound of Formula (I), or a pharmaceutically acceptable salt thereof, and an anti-cotinine antibody, or antigen-binding fragment thereof, wherein the disease or disorder is selected from a cancer.

In a further embodiment, the compound and the antibody, or antigen-binding fragment thereof, are administered simultaneously. In a further embodiment, the compound and the antibody, or antigen-binding fragment thereof, are administered simultaneously from a single composition, including as a fixed-dose composition or by pre-mixing the compound and the antibody, or antigen-binding fragment thereof, prior to administration. For example, the compound and the antibody, or antigen-binding fragment thereof, can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the antibody, or antigen-binding fragment thereof, are administered simultaneously from two separate compositions.

In a further embodiment, the compound and the antibody, or antigen-binding fragment thereof, are administered sequentially.

In certain embodiments, the compound and the antibody, or antigen-binding fragment thereof, whether administered simultaneously or sequentially, may be administered by the same route or may be administered by different routes. In one embodiment, the compound and the antibody, or antigen-binding fragment thereof, are both administered intravenously or subcutaneously, in the same composition or in separate compositions. In another embodiment, the compound is administered orally and the antibody or antigen-binding fragment thereof is administered intravenously or subcutaneously.

In a further embodiment, the compound and the antibody, or antigen-binding fragment thereof, are administered in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are present as a combination in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are administered at a dosage of compound of 0.0001 mg/kg to 1 mg/kg and antibody of 0.01 mg/kg to 100 mg/kg. For example, in a further embodiment, the compound is administered at a dosage of about 0.0001 mg/kg to about 0.0002 mg/kg, about 0.0002 mg/kg to about 0.0003 mg/kg, about 0.0003 mg/kg to about 0.0004 mg/kg, about 0.0004 mg/kg to about 0.0005 mg/kg, about 0.0005 mg/kg to about 0.001 mg/kg, about 0.001 mg/kg to about 0.002 mg/kg, about 0.002 mg/kg to about 0.003 mg/kg, about 0.003 mg/kg to about 0.004 mg/kg, about 0.004 mg/kg to about 0.005 mg/kg, about 0.005 mg/kg to about 0.01 mg/kg, about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, and/or about 0.5 mg/kg to about 1 mg/kg, and the antibody, or antigen-binding fragment thereof, is administered at a dosage of about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, and/or about 90 mg/kg to about 100 mg/kg.

In a further embodiment, the compound and the antibody, or antigen-binding fragment thereof, are administered at a dosage of compound of 0.007 mg to 70 mg and antibody of 0.7 mg to 7000 mg. For example, in a further embodiment, the compound is administered at a dosage of about 0.007 mg to about 0.01 mg, about 0.01 mg to about 0.02 mg, about 0.02 mg to about 0.03 mg, about 0.03 mg to about 0.04 mg, about 0.04 mg to about 0.05 mg, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, and/or about 60 mg to about 70 mg, and the antibody, or antigen-binding fragment thereof, is administered at a dosage of about 0.7 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 100 mg, about 100 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, and/or about 6500 mg to about 7000 mg.

In a further embodiment, the compound and the antibody, or antigen-binding fragment thereof, are administered in a molar ratio and/or dosage as described herein once every week, once every two weeks, once every three weeks, once every four weeks, once every five weeks, or once every six weeks for a period of one week to one year, such as a period of one week, one month, two months, three months, four months, five months, six months, seven months, eight months, nine months, ten months, eleven months, or twelve months.

In a further embodiment, the present disclosure provides a therapeutically effective amount of the compound of Formula (I), or a pharmaceutically acceptable salt thereof, and an anti-cotinine antibody, or antigen-binding fragment thereof for use in therapy. The compound of Formula (I), or a pharmaceutically acceptable salt thereof, and anti-cotinine antibody, or antigen-binding fragment thereof can be used in treating or preventing a disease or disorder selected from a cancer.

In a further embodiment, the present disclosure provides a therapeutically effective amount of the compound of Formula (I), or a pharmaceutically acceptable salt thereof, and an anti-cotinine antibody, or antigen-binding fragment thereof for the manufacture of a medicament. The medicament can be used in treating or preventing a disease or disorder selected from a cancer.

In a further embodiment, the disease or disorder is mediated by PSMA and/or is associated with PSMA-positive pathogenic cells. In a further embodiment, PSMA-positive cell types are identified by testing for expression of PSMA by immunohistochemistry or flow cytometry.

In a further embodiment, the disease or disorder is a cancer selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, renal cell cancer, bladder cancer, or breast cancer, preferably mCRPC, breast cancer, lung cancer, colorectal cancer, or renal cell cancer.

In a further embodiment, the disease or disorder is a solid tumor. In a further embodiment, the disease or disorder is a solid tumor selected from lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, renal cell cancer, bladder cancer, or breast cancer, preferably mCRPC, breast cancer, lung cancer, colorectal cancer, or renal cell cancer.

In a further embodiment, the disease or disorder is a PD-1 relapsed or refractory cancer, such as a PD-1 relapsed or refractory lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, renal cell cancer, bladder cancer, or breast cancer, preferably the PD-1 relapsed or refractory cancer is a mCRPC, breast cancer, lung cancer, colorectal cancer, or renal cell cancer.

In a further embodiment, the disease or disorder is a non-solid cancer. In a further embodiment, the disease or disorder is a leukemia, a lymphoma, or a myeloma.

In one embodiment, the present disclosure provides a method of increasing antibody-dependent cell cytotoxicity (ADCC) of PSMA-expressing cells comprising contacting the cells with an effective amount of the compound of Formula (I), or pharmaceutically acceptable salt thereof, and an anti-cotinine antibody, or antigen-binding fragment thereof, wherein the PSMA-binding moiety of the compound binds the PSMA expressed on the cells.

In one embodiment, the present disclosure provides a method of increasing antibody dependent cellular phagocytosis (ADCP) of PSMA-expressing cells comprising contacting the cells with an effective amount of the compound of Formula (I), or pharmaceutically acceptable salt thereof, and an anti-cotinine antibody, or antigen-binding fragment thereof, wherein the PSMA-binding moiety of the compound binds the PSMA expressed on the cells.

In one embodiment, the present disclosure provides a method of increasing complement dependant cytotoxicity (CDC) of PSMA-expressing cells comprising contacting the cells with an effective amount of the compound of Formula (I), or pharmaceutically acceptable salt thereof, and an anti-cotinine antibody, or antigen-binding fragment thereof, wherein the PSMA-binding moiety of the compound binds the PSMA expressed on the cells.

In one embodiment, the present disclosure provides a method of conditioning a patient for therapy with a chimeric antigen receptor (CAR) T cell therapy, comprising administering to a patient an effective amount of the compound of Formula (I), or pharmaceutically acceptable salt thereof, and an anti-cotinine antibody, or antigen-binding fragment thereof. In some embodiments, the compound of Formula (I), or pharmaceutically acceptable salt thereof, and an anti-cotinine antibody, or antigen-binding fragment thereof are administered in combination with the CAR-T cell therapy. A compound of Formula (I), or pharmaceutically acceptable salt thereof, and an anti-cotinine antibody, or antigen-binding fragment thereof may be administered as a conditioning therapy or combination therapy to improve efficacy in treatment of solid tumor cancers. In other embodiments, a compound of Formula (I), or pharmaceutically acceptable salt thereof, and an anti-cotinine antibody, or antigen-binding fragment thereof may be administered as a neoadjuvant treatment for other therapies, including but not limited to immunotherapy, surgical resection, radiation, and/or chemotherapy.

In one embodiment, the present disclosure provides a method of depleting PSMA-expressing cells comprising contacting the cells with the compound of Formula (I), or pharmaceutically acceptable salt thereof, and an anti-cotinine antibody, or antigen-binding fragment thereof, wherein the PSMA-binding moiety of the compound binds the PSMA expressed on the cells.

In a further embodiment, the PSMA-expressing cells are pathogenic cells.

In a further embodiment, the pathogenic cell is a tumor cell or cancer cell, or endothelial cell.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, renal cell cancer cells, bladder cancer cells, or breast cancer cells. In a further embodiment, the tumor cells or cancer cells are mCRPC cells, breast cancer cells, lung cancer cells, colorectal cancer cells, or renal cell cancer cells.

In a further embodiment, the pathogenic cell is an endothelial cell associated with tumor neovasculature.

### Combination Therapies

The compounds of the invention may be employed alone or in combination with other therapeutic agents. Combination therapies according to the present invention thus comprise the administration of at least one compound of Formula (I) or a pharmaceutically acceptable salt thereof, and the use of at least one other pharmaceutically active agent. The compounds of the invention and the other pharmaceutically active agents may be administered together in a single pharmaceutical composition or separately and, when administered separately this may occur simultaneously or sequentially in any order. The amounts of the compounds of the invention and the other pharmaceutically active agents and the relative timings of administration will be selected in order to achieve the desired combined therapeutic effect.

It will be appreciated that when the compound of the present invention is administered in combination with one or more other therapeutically active agents normally administered by the inhaled, intravenous, oral, intranasal, ocular topical or other route, that the resultant pharmaceutical composition may be administered by the same route. Alternatively, the individual components of the composition may be administered by different routes.

In one embodiment, the compounds and pharmaceutical composition disclosed herein are used in combination with, or include, one or more additional therapeutic agents. In a further embodiment, the additional therapeutic agent is a checkpoint inhibitor or an immune modulator.

In a further embodiment, the checkpoint inhibitor is selected from a PD-1 inhibitor (e.g., an anti-PD-1 antibody including, but not limited to, pembrolizumab, nivolumab, cemiplimab, or dostarlimab), a PD-L1 inhibitor (e.g., an anti-PD-L1 antibody including, but not limited to, atezolizumab, avelumab, or durvalumab), or a CTLA-4 inhibitor (e.g., an anti-CTLA-4 antibody including, but not limited to, ipilimumab or tremilumumab).

In a further embodiment, the checkpoint inhibitor is selected from a CD226 axis inhibitor, including but not limited to a TIGIT inhibitor (e.g., an anti-TIGIT antibody), a CD96 inhibitor (e.g., an anti-CD96 antibody), and/or a PVRIG inhibitor (e.g., an anti-PVRIG antibody).

In a further embodiment, the immune modulator is an ICOS agonist (e.g., an anti-ICOS antibody including, but not limited to feladilimab), a PARP inhibitor (e.g., niraparib, olaparib), or a STING agonist.

### Pharmaceutical Compositions, Dosages, and Dosage Forms

For the purposes of administration, in certain embodiments, the ARMs described herein are administered as a raw chemical or are formulated as pharmaceutical compositions. Pharmaceutical compositions disclosed herein include an ARM and one or more of: a pharmaceutically acceptable carrier, diluent or excipient. An ARM is present in the composition in an amount which is effective to treat a particular disease, disorder or condition of interest. The activity of the ARM can be determined by one skilled in the art, for example, as described in the biological assays described below. Appropriate concentrations and dosages can be readily determined by one skilled in the art. In certain embodiments, the ARM is present in the pharmaceutical composition in an amount from about 25 mg to about 500 mg. In certain embodiments, the ARM is present in the pharmaceutical composition in an amount of about 0.01 mg to about 300 mg. In certain embodiments, ARM is present in the pharmaceutical composition in an amount of about 0.01 mg, 0.1 mg, 1 mg, 5 mg, 10 mg, 25 mg, 50 mg, 100 mg, 200 mg, 300 mg, 400 mg or about 500 mg.

Administration of the compounds of the invention, or their pharmaceutically acceptable salts, in pure form or in an appropriate pharmaceutical composition, is carried out via any of the accepted modes of administration of agents for serving similar utilities. The pharmaceutical compositions of the invention are prepared by combining a compound of the invention with an appropriate pharmaceutically acceptable carrier, diluent or excipient, and in specific embodiments are formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral (e.g., intramuscular, subcutaneous, intravenous, or intradermal), sublingual, buccal, rectal, vaginal, and intranasal. Pharmaceutical compositions of the invention are formulated so as to allow the active ingredients contained therein to be bioavailable upon administration of the composition to a patient.

Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia. College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings described herein.

The pharmaceutical compositions disclosed herein are prepared by methodologies well known in the pharmaceutical art. For example, in certain embodiments, a pharmaceutical composition intended to be administered by injection is prepared by combining a compound of the invention with sterile, distilled water so as to form a solution. In some embodiments, a surfactant is added to facilitate the formation of a homogeneous solution or suspension. Surfactants are compounds that non-covalently interact with the compound of the invention so as to facilitate dissolution or homogeneous suspension of the compound in the aqueous delivery system.

Traditional antibody therapeutics have several disadvantages that are addressed by the ARMs approach described herein including difficulties in managing adverse events via adjusting dose and dose frequency of administration, challenges in generating antibodies to certain classes of drug targets (e.g., GPCRs, ion channels, and enzymes), and a new cell line for development is required for each new antibody which can be slow and costly. Moreover, different formats of biologics (e.g., bispecifics) can be challenging to manufacture. In contrast, the ARMs approach provides the following advantages: uniting the pharmacology of antibodies with the dose-control of small molecules, dose controlled PK/PD allowing temporal cell depletion, simpler multimerization, and rapid reversal of cell depletion through dosing of the antibody-binding component (e.g., cotinine hapten) which can uncouple therapeutic effects from potential adverse events.

### EXAMPLES

The following examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made. References to preparations carried out in a similar manner to, or by the general method of, other preparations, may encompass variations in routine parameters such as time, temperature, workup conditions, minor changes in reagent amounts etc. Chemical names for all title compounds were generated using ChemDraw Plug-in version 16.0.1.13c (90) or ChemDraw desktop version 16.0.1.13 (90). A person of ordinary skill in the art will recognize that compounds of the invention may have alternative names when different naming software is used.

### Compound Synthesis

The compounds according to Formulae (I) and (II) are prepared using conventional organic synthetic methods. A suitable synthetic route is depicted below in the following general reaction schemes. All the starting materials are commercially available or are readily prepared from commercially available starting materials by those of skill in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Organic Synthesis (4th ed.), John Wiley & Sons, NY (2006). In some instances, a substituent may be specifically selected to be reactive under the reaction conditions used. Under these circumstances, the reaction conditions convert the selected substituent into another substituent that is either useful as an intermediate compound or is a desired substituent in a target compound.

### Scheme 1

### Intermediate 1: (2S,3S)-1-Methyl-5-oxo-2-(pyridin-3-yl)pyrrolidine-3-carboxylic acid

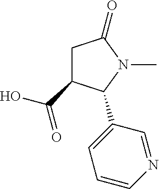 [SMILES: CN1C(=O)C[C@H](C(=O)O)[C@H]1c1cccnc1]

Commercially available, racemic trans-4-cotininecarboxylic acid (304 g, 1.38 mol) was purified by chiral prep HPLC (61 injections) on Chiralpak 1A 20u 101×210 mm at 500 mL/min eluting with 50% acetonitrile in methanol containing 0.1% formic acid. The desired fractions were collected and were concentrated at 45° C. The solid residue was stirred in acetonitrile, was filtered, and was dried under reduced pressure for 18 h to provide the title compound as a white solid (143.6 g, 652 mmol, 94.5% yield). Analytical chiral HPLC: 95% ee at ret. time 2.5 min, Chiralpak AD-H 5u 4.6×150 mm, methanol with 0.1% formic acid at 1.0 mL/min; Alpha D=+58 deg (c=0.3, CH₃OH); VCD analysis was used to assign absolute stereochemistry. LC-MS m/z 221.1 (M+H)⁺. ¹H NMR (400 MHZ, DMSO-d₆) δ ppm 2.48-2.49 (m, 2H) 2.53-2.61 (m, 1H) 2.71-2.80 (m, 1H) 3.06-3.15 (m, 1H) 3.34 (br s, 1H) 4.79 (d, J=6.3 Hz, 1H) 7.35-7.57 (m, 1H) 7.74 (dt, J=7.9, 2.0 Hz, 1H) 8.54 (d, J=1.8 Hz, 1H) 8.57 (dd, J=4.7, 1.7 Hz, 1H) 12.78 (br s, 1H).

### Intermediate 2: (1R,4r)-4-(4-(((1S,4R)-4-(2-((2S,3S)-1-Methyl-5-oxo-2-(pyridin-3-yl)pyrrolidine-3-carboxamido)ethoxy)cyclohexyl)oxy)butanamido)cyclohexane-1-carboxylic acid, Hydrochloride salt

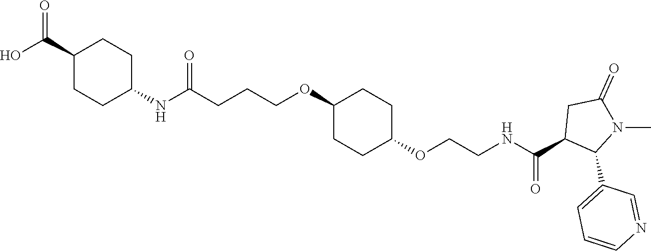 [SMILES: CN1C(=O)C[C@H](C(=O)NCCO[C@H]2CC[C@H](OCCCC(=O)N[C@H]3CC[C@H](C(=O)O)CC3)CC2)[C@H]1c1cccnc1]

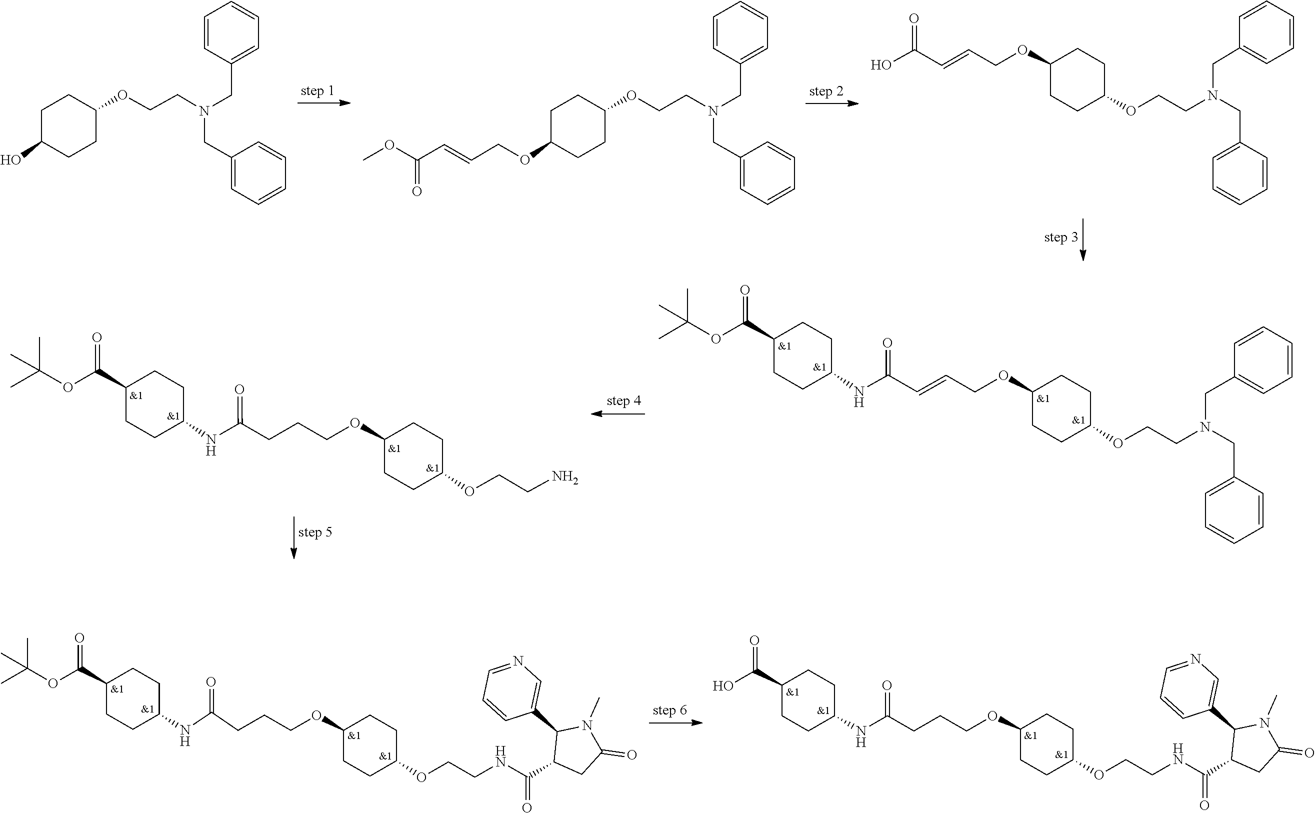

### Step 1: methyl (E)-4-(((1r,4r)-4-(2-(Dibenzylamino)ethoxy)cyclohexyl)oxy)but-2-enoate

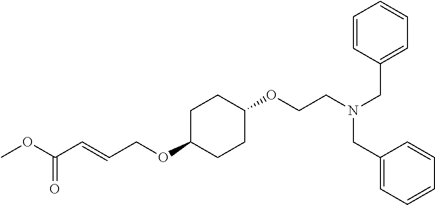 [SMILES: COC(=O)/C=C/CO[C@H]1CC[C@H](OCCN(Cc2ccccc2)Cc2ccccc2)CC1]

To a stirred solution of (1r,4r)-4-(2-(dibenzylamino)ethoxy)cyclohexan-1-ol (250 g, 736 mmol) in toluene (2500 mL) were added methyl but-2-ynoate (140 g, 1423 mmol), triphenylphosphine (19.32 g, 73.6 mmol), and acetic acid (16.86 mL, 295 mmol) at RT and the resulting solution was stirred at 115° C. for 16 h. The reaction mixture was cooled to room temperature and concentrated under reduced pressure to the crude compound. The crude compound was adsorbed on silicagel (500 g, 60-120 mesh), and purified by manual column chromatography (1.5 kg, 100-200 mesh) eluted with 15% EtOAc in pet-ether to afford methyl (E&Z)-4-(((1r,4r)-4-(2-(dibenzylamino)ethoxy)cyclohexyl)oxy)but-2-enoate (350 g) as a mixture of E/Z isomers (52.48% and 21.15%). To separate both isomers, the compound was adsorbed on silica gel (500 g, 100-200 mesh), and purified by manual column chromatography (1.5 kg, 100-200 mesh) eluted with 15% EtOAc in pet-ether to afford the title compound (240 g, 463 mmol, 62.9% yield, 84.45% purity) as a pale yellow liquid. LC-MS m/z 438.3 (M+H)⁺.

### Step 2: (E)-4-(((1,4-trans)-4-(2-(Dibenzylamino)ethoxy)cyclohexyl)oxy)but-2-enoic acid

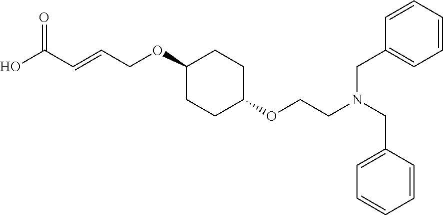 [SMILES: O=C(O)/C=C/CO[C@H]1CC[C@H](OCCN(Cc2ccccc2)Cc2ccccc2)CC1]

Methyl (E)-4-(((1,4-trans)-4-(2-(dibenzylamino)ethoxy)cyclohexyl)oxy)but-2-enoate (9.03 g, 20.64 mmol) was dissolved in tetrahydrofuran (THF) (25 mL) and aqueous 5.089 Molar sodium hydroxide (4.87 mL, 24.76 mmol) was added. The homogenous pale-yellow reaction was heated at reflux for 1 hour. Additional 5.089 M sodium hydroxide (1.217 mL, 6.19 mmol) was added and the reaction was refluxed for 50 minutes. The reaction was cooled over 60 minutes and concentrated in vacuo. The residue was azeotroped twice with toluene in order to aid in removal of water. The residue was pumped under high vacuum to afford the title compound (9.9 g, 22.17 mmol, 107% yield, 82% purity, E/Z mixture) as a yellow solid. LC-MS m/z 424.4 (M+H)⁺.

### Step 3: tert-butyl (1R,4r)-4-((E)-4-(((1r,4R)-4-(2-(Dibenzylamino)ethoxy) cyclohexyl)oxy)but-2-enamido)cyclohexane-1-carboxylate

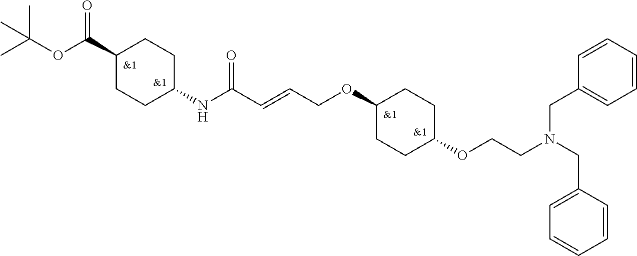 [SMILES: CC(C)(C)OC(=O)[C@H]1CC[C@H](NC(=O)/C=C/CO[C@H]2CC[C@H](OCCN(Cc3ccccc3)Cc3ccccc3)CC2)CC1]

(E)-4-(((1,4-trans)-4-(2-(dibenzylamino)ethoxy)cyclohexyl)oxy)but-2-enoic acid, sodium salt (9.2 g, 20.60 mmol) was suspended in dry DMF (40 ml) with stirring. HATU (8.62 g, 22.66 mmol) was added as a solid and a partially dissolved mixture was observed. The mixture was stirred for 30 minutes to give a partially dissolved greenish solution. tert-butyl (1,4-trans)-4-aminocyclohexane-1-carboxylate (4.11 g, 20.60 mmol) was added as a solution in DMF (10 ml) followed by addition of a solution of DIEA (10.80 mL, 61.8 mmol) in DMF (10 ml). An additional 10 ml of DMF was added and the heterogeneous mixture was stirred for 15 hours at room temperature. Additional HATU (1.724 g, 4.53 mmol) was added and the almost homogeneous reaction was stirred for 60 minutes. The cloudy reaction was stirred for an additional 60 minutes. The reaction was diluted with 200 ml of EtOAc and 200 ml of water and stirred for 10 minutes. The resulting homogeneous biphasic mixture was transferred to a separatory funnel and the layers were separated. The aqueous layer was extracted twice more with 150 ml EtOAc and the combined EtOAc layers were washed 4× with water and 2× with saturated NaCl in order to remove DMF. The EtOAc extracts were dried over sodium sulfate, filtered, and concentrated in vacuo, and pumped under high vacuum to give an orange syrup which was purified via silica-gel chromatography (Isco Combiflash, 330 gram gold column, 0-80% EtOAc:heptane over 45 minutes, 150 ml/min flow rate, loaded as a solution in DCM) to give the title compound (4.55 g, 7.52 mmol, 36.5% yield) as a white foamy solid. LC-MS m/z 605.5 (M+H)⁺.

### Step 4: tert-butyl (1R,4r)-4-(4-(((1r,4R)-4-(2-Aminoethoxy)cyclohexyl)oxy)butanamido)cyclohexane-1-carboxylate

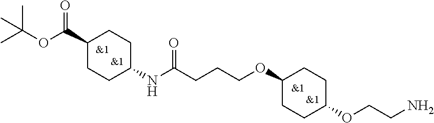 [SMILES: CC(C)(C)OC(=O)[C@H]1CC[C@H](NC(=O)CCCO[C@H]2CC[C@H](OCCN)CC2)CC1]

A 500 ml 3-necked RB flask was charged with tert-butyl (1R,4r)-4-((E)-4-(((1r,4R)-4-(2-(dibenzylamino)ethoxy)cyclohexyl)oxy)but-2-enamido)cyclohexane-1-carboxylate (6.40 g, 10.58 mmol) and isopropanol (120 mL) and the suspension was stirred until a homogeneous solution was obtained. 10% wet Pd—C (0.640 g, 6.01 mmol) was added and the flask was evacuated and placed under 2 balloons of hydrogen attached to the end necks of the flask. The middle neck was covered with a rubber septum. The reaction was stirred at room temperature for 16 hours. The reaction was degassed and filtered 2× through celite. The filtrate was concentrated in vacuo and pumped under high vacuum to give a waxy grey solid with a slight odor of isopropanol. The waxy solid was dissolved in DCM and concentrated in vacuo at 54 degrees C. for 20 minutes in order to aid in removal of isopropanol. The residue was pumped under high vacuum to afford the title compound (4.44 g, 10.41 mmol, 98% yield) as a waxy grey solid. LC-MS m/z 427.4 (M+H)⁺.

### Step 5: tert-butyl (1R,4r)-4-(4-(((1S,4R)-4-(2-((2S,3S)-1-Methyl-5-oxo-2-(pyridin-3-yl)pyrrolidine-3-carboxamido)ethoxy)cyclohexyl)oxy)butanamido)cyclohexane-1-carboxylate

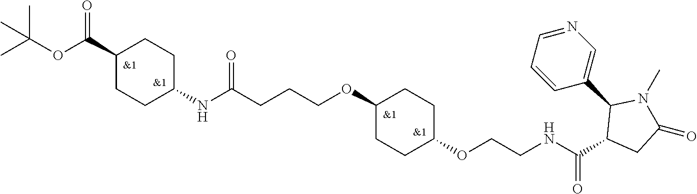 [SMILES: CN1C(=O)C[C@H](C(=O)NCCO[C@H]2CC[C@H](OCCCC(=O)N[C@H]3CC[C@H](C(=O)OC(C)(C)C)CC3)CC2)[C@H]1c1cccnc1]

(2S,3S)-1-Methyl-5-oxo-2-(pyridin-3-yl)pyrrolidine-3-carboxylic acid (Intermediate 1) (2.287 g, 10.38 mmol) was suspended in 30 ml DCM with stirring in a 500 ml RB flask at room temperature. HATU (4.34 g, 11.42 mmol) was added and the suspension was stirred for 15 minutes. A solution of tert-butyl (1R,4r)-4-(4-(((1r,4R)-4-(2-aminoethoxy)cyclohexyl)oxy)butanamido)cyclohexane-1-carboxylate (4.43 g, 10.38 mmol) in DCM (20 ml) was added dropwise via pipet over 15 minutes. After addition was complete, a solution of DIEA (5.44 mL, 31.2 mmol) in DCM (10 ml) was added dropwise over 10 minutes and the resulting homogeneous dark solution was stirred at room temperature for 16 hours. The reaction was concentrated in vacuo in order to remove DCM and DIEA. The residue was dissolved in 100 ml DCM and transferred to a separatory funnel. 20 ml of saturated sodium bicarbonate was added The layers were separated and the DCM layer was washed with saturated NaCl, dried over sodium sulfate, filtered, concentrated in vacuo, and pumped under high vacuum to give an orange syrup which was purified via silica gel chromatography (Isco Combiflash, 0-10% MeOH:DCM over 60 minutes, 330 gram gold column, 150 ml/min flow rate, loaded as a solution in 30 ml DCM) to afford the title compound (4.13 g, 6.57 mmol, 63.2% yield) as a white solid. LC-MS m/z 629.3 (M+H)⁺.

### Step 6: (1R,4r)-4-(4-(((1S,4R)-4-(2-((2S,3S)-1-Methyl-5-oxo-2-(pyridin-3-yl)pyrrolidine-3-carboxamido)ethoxy)cyclohexyl)oxy)butanamido)cyclohexane-1-carboxylic acid, Hydrochloride salt

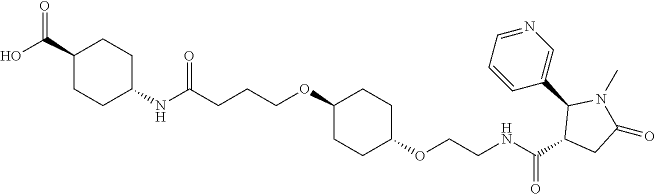 [SMILES: CN1C(=O)C[C@H](C(=O)NCCO[C@H]2CC[C@H](OCCCC(=O)N[C@H]3CC[C@H](C(=O)O)CC3)CC2)[C@H]1c1cccnc1]

tert-Butyl (1R,4r)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylate (4.13 g, 6.57 mmol) was dissolved in dry 1,4-dioxane (13 ml) with stirring in a 250 ml RB flask. 4 M anhydrous HCl (39 mL, 156 mmol) in 1,4-dioxane was added and the mixture was stirred at room temperature. Upon HCl addition, an insoluble oil was observed. The mixture was stirred for 90 minutes at room temperature. The reaction was concentrated in vacuo (at 60 degrees bath temperature) and pumped under high vacuum for 15 hours to afford the title compound (4.187 g, 6.87 mmol, 105% yield) as a white solid. LC-MS m/z 573.4 (M+H)⁺.

### Intermediate 3: 1-((2S,3S)-1-Methyl-5-oxo-2-(pyridin-3-yl)pyrrolidin-3-yl)-1-oxo-5,8,11,14,17,20,23,26,29,32,35,38-dodecaoxa-2-azahentetracontan-41-oic acid, Ammonia salt

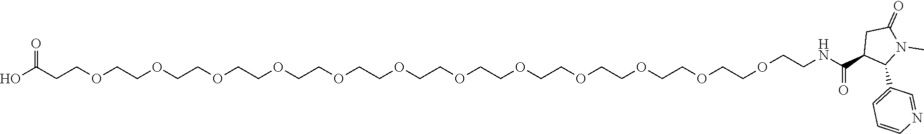 [SMILES: CN1C(=O)C[C@H](C(=O)NCCOCCOCCOCCOCCOCCOCCOCCOCCOCCOCCOCCOCCC(=O)O)[C@H]1c1cccnc1]

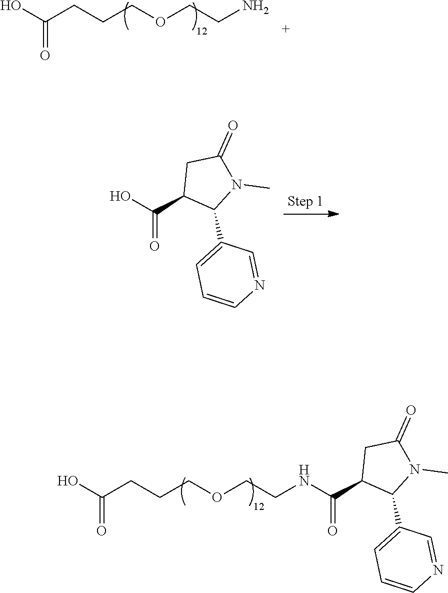

### Step 1: 1-((2S,3S)-1-Methyl-5-oxo-2-(pyridin-3-yl)pyrrolidin-3-yl)-1-oxo-5,8,11,14,17,20,23,26,29,32,35,38-dodecaoxa-2-azahentetracontan-41-oic acid, Ammonia salt

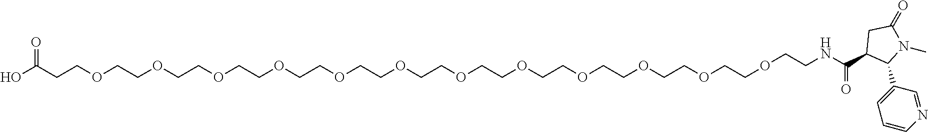 [SMILES: CN1C(=O)C[C@H](C(=O)NCCOCCOCCOCCOCCOCCOCCOCCOCCOCCOCCOCCOCCC(=O)O)[C@H]1c1cccnc1]

In a round bottom flask, (2S,3S)-1-methyl-5-oxo-2-(pyridin-3-yl)pyrrolidine-3-carboxylic acid (Intermediate 1) (0.357 g, 1.619 mmol) was dissolved in acetonitrile (10 ml). TSTU (0.487 g, 1.619 mmol) and DIPEA (1.131 ml, 6.48 mmol) were then added and the reaction was stirred at RT. Upon activation of the acid, 1-amino-3,6,9, 12, 15, 18,21,24,27,30,33,36-dodecaoxanonatriacontan-39-oic acid (1 g, 1.619 mmol) was added as a solid and N,N-dimethylformamide (DMF) (3 ml) was added to aid in the solubility of the acid. The reaction was stirred at RT and monitored by LCMS. Upon completion, the reaction was concentrated to afford a viscous oil. The crude material was purified by reverse phase chromatography (loading as a solution in ˜6 mL of 10 mM aqueous ammonium bicarbonate in H20 adjusted to pH 10 with ammonia, C18 100 g Gold column, 60 mL/min, gradient 5-50% 10 mM aqueous ammonium bicarbonate in H20 adjusted to pH 10 with ammonia to MeCN over 23 min) to afford the title compound (1.3 g, 1.351 mmol, 83% yield) as a colorless oil (87% purity by NMR, the material contains CH₂Cl₂). LC-MS m/z 820.1 (M+H)⁺.

### Intermediate 4: Tri-tert-butyl (5S,8S,22S,26S)-1-amino-5,8-dibenzyl-4,7,10,19,24-pentaoxo-3,6,9,18,23,25-hexaazaoctacosane-22,26,28-tricarboxylate

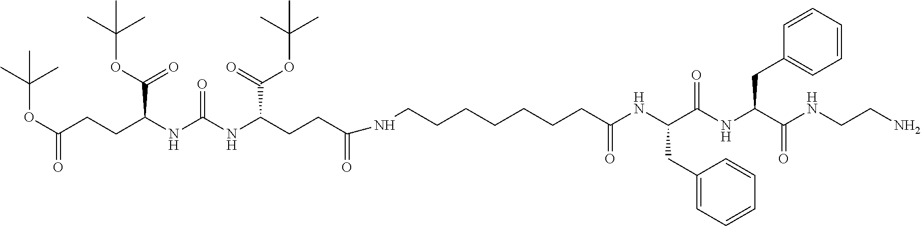 [SMILES: CC(C)(C)OC(=O)CC[C@H](NC(=O)N[C@@H](CCC(=O)NCCCCCCCC(=O)N[C@@H](Cc1ccccc1)C(=O)N[C@@H](Cc1ccccc1)C(=O)NCCN)C(=O)OC(C)(C)C)C(=O)OC(C)(C)C]

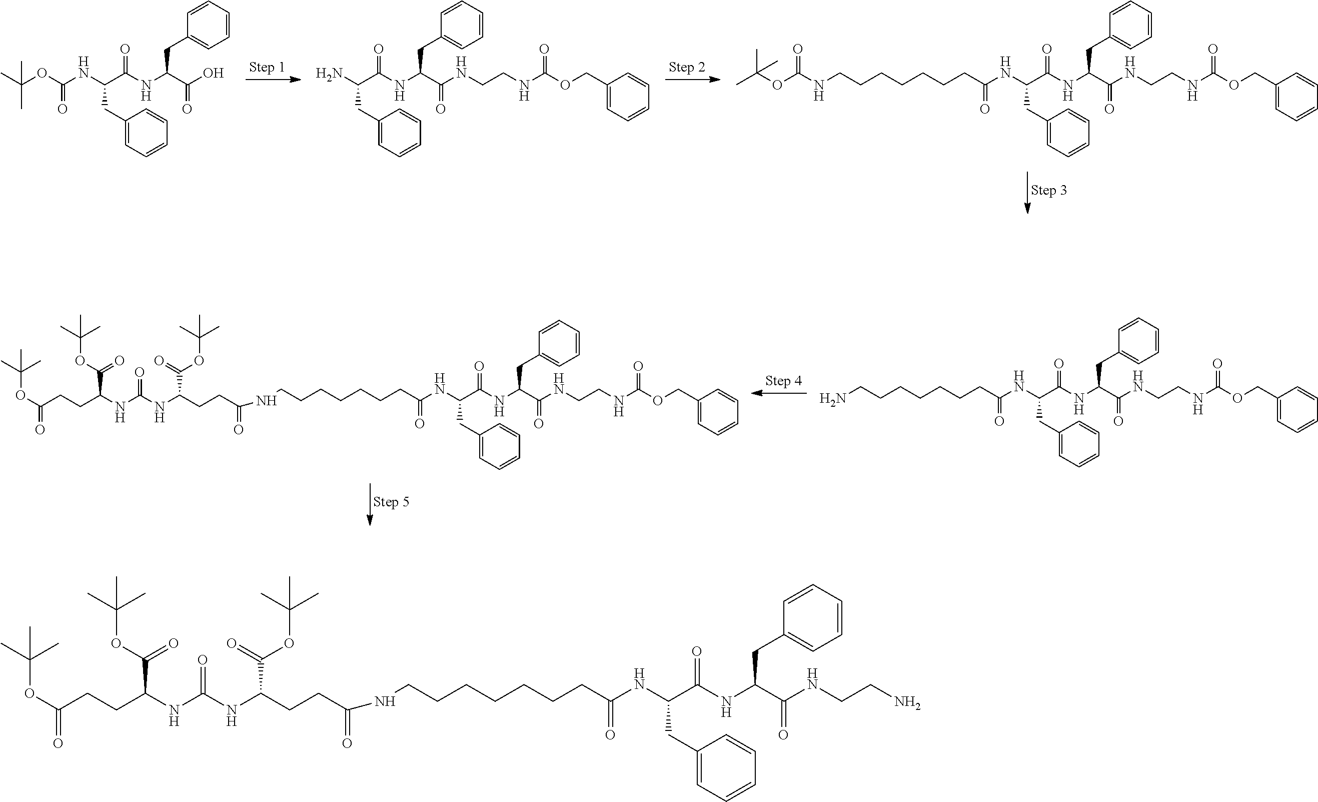

### Step 1: Benzyl (2-((S)-2-((S)-2-amino-3-phenylpropanamido)-3-phenyl propanamido)ethyl)carbamate

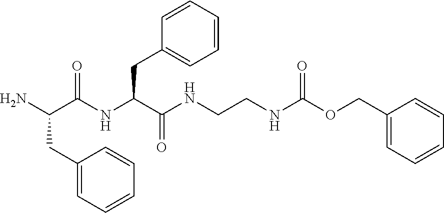 [SMILES: N[C@@H](Cc1ccccc1)C(=O)N[C@@H](Cc1ccccc1)C(=O)NCCNC(=O)OCc1ccccc1]

To a solution of (tert-butoxycarbonyl)-L-phenylalanyl-L-phenylalanine (2 g, 4.85 mmol), benzyl (2-aminoethyl)carbamate (0.942 g, 4.85 mmol), and HATU (2.397 g, 6.30 mmol) in anhydrous N,N-Dimethylformamide (DMF) (9.70 ml) was added N, N-diisopropylethylamine (1.694 ml, 9.70 mmol). The reaction was stirred at RT for 1 h at which point the reaction had solidified. DCM (20 mL) was added and let continue at RT (20 h). Concentrated to remove DCM, then added 20 mL H₂O. Collected solid via filtration, washing with additional H₂O (2×10 mL). Dried in vacuum oven at 50° C. over weekend. Extracted filtrate with 3×30 mL EtOAc. Washed organics with 2×30 mL sat NaHCO3, 30 ml brine. Dried over sodium sulfate, filtered, and concentrated. To a suspension of the resultant residue in DCM (20 ml) was added TFA (3.74 ml, 48.5 mmol). The reaction became a clear yellow solution and was stirred at RT 22.5 h then concentrated and redissolved in 10% MeOH/DCM (150 mL). Added sat NaHCO3 to pH=8-10, then extracted aqueous portion 3×50 mL with 10% MeOH/DCM. Dried combined organics over sodium sulfate, filtered, and concentrated. Purified on 80 g ISCO gold silica gel column, eluting with 0-10% MeOH/DCM. Desired fractions were combined and concentrated, resulting in the title compound (1.854 g, 78.3% yield) as an off white solid. LC-MS m/z (M+H)⁺ 489.1

### Step 2: tert-butyl ((9S,12S)-9,12-dibenzyl-3,8,11,14-tetraoxo-1-phenyl-2-oxa-4,7,10,13-tetraazahenicosan-21-yl)carbamate

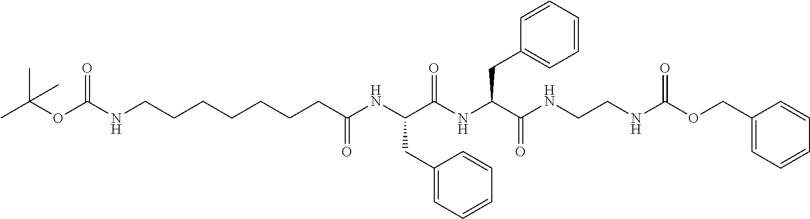 [SMILES: CC(C)(C)OC(=O)NCCCCCCCC(=O)N[C@@H](Cc1ccccc1)C(=O)N[C@@H](Cc1ccccc1)C(=O)NCCNC(=O)OCc1ccccc1]

To a solution of benzyl (2-((S)-2-((S)-2-amino-3-phenylpropanamido)-3-phenylpropanamido)ethyl)carbamate (1 g, 2.047 mmol), 8-((tert-butoxycarbonyl)amino)octanoic acid (0.557 g, 2.149 mmol), and HATU (1.012 g, 2.66 mmol) in anhydrous Dichloromethane (DCM) (20.47 ml) was added N, N-diisopropylethylamine (0.715 ml, 4.09 mmol). The reaction was stirred at RT for 1 h, then DCM (30 mL) was added to facilitate stirring. Let continue at RT

4 d. Concentrated to remove DCM. Washed solid with 2×20 mL sat NaHCO₃, 20 mL H₂O, dried over anhydrous sodium sulfate, filtered, and concentrated resulting in the title compound (2.0125 g, >theoretical) as an orange solid. LC-MS m/z (M+H)⁺ 730.3.

### Step 3: Benzyl (2-((S)-2-((S)-2-(8-aminooctanamido)-3-phenylpropanamido)-3-phenylpropanamido)ethyl)carbamate

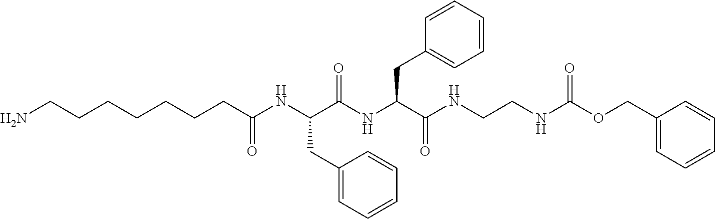 [SMILES: NCCCCCCCC(=O)N[C@@H](Cc1ccccc1)C(=O)N[C@@H](Cc1ccccc1)C(=O)NCCNC(=O)OCc1ccccc1]

To a solution/suspension of benzyl ((15S,18S)-15,18-dibenzyl-2,2-dimethyl-4,13,16,19-tetraoxo-3-oxa-5,14,17,20-tetraazadocosan-22-yl)carbamate (1.494 g, 2.047 mmol) in Dichloromethane (DCM) (20.47 mL) was added TFA (1.58 ml, 20.5 mmol). The reaction was stirred at RT for 24 h. Additional TFA (0.473 mL, 6.14 mmol) was added, and the mixture was stirred at RT for 4 h. Water (20 mL) was added, and the resultant beige precipitate was collected via filtration, washed with DCM (2×10 mL) and water (2×10 mL), and dried in a 50° C. drying oven to provide the title compound as a beige solid (1.44 g, 2.46 mmol, 95.0% yield). LC-MS m/z 630.3 (M+H)⁺.

### Step 4: Tri-tert-butyl (9S,12S,26S,30S)-9,12-dibenzyl-3,8,11,14,23,28-hexaoxo-1-phenyl-2-oxa-4,7,10,13,22,27,29-heptaazadotriacontane-26,30,32-tricarboxylate

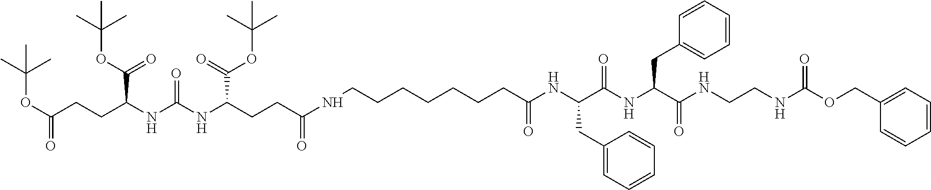 [SMILES: CC(C)(C)OC(=O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=O)OCc1ccccc1)C(=O)OC(C)(C)C)C(=O)OC(C)(C)C]

To a solution of (S)-5-(tert-butoxy)-4-(3-((S)-1,5-di-tert-butoxy-1,5-dioxopentan-2-yl)ureido)-5-oxopentanoic acid (269 mg, 0.551 mmol), benzyl (2-((S)-2-((S)-2-(8-aminooctanamido)-3-phenylpropanamido)-3-phenylpropanamido)ethyl)carbamate 2,2,2-trifluoroacetate (410 mg, 0.551 mmol), and HATU (272 mg, 0.717 mmol) in N,N-Dimethylformamide (3.7 mL) was added DIPEA (289 μl, 1.65 mmol). The mixture was stirred at RT for 2 h. The mixture was diluted with EtOAc (50 mL) and washed with saturated aqueous sodium bicarbonate (2×30 mL) and brine (30 mL). To the gelatinous organic layer was added MeOH (50 mL). The resultant white precipitate was removed via filtration. The filtrate was dried over anhydrous sodium sulfate, filtered, and concentrated. The resultant residue was purified by ISCO CombiFlash® chromatography eluting with a gradient of 0 to 10% methanol in dichloromethane to provide the title compound (469.3 mg, 0.444 mmol, 77.3% yield). LC-MS m/z 1100.8 (M+H)⁺.

### Step 5: Tri-tert-butyl (5S,8S,22S,26S)-1-amino-5,8-dibenzyl-4,7,10,19,24-pentaoxo-3,6,9,18,23,25-hexaazaoctacosane-22,26,28-tricarboxylate

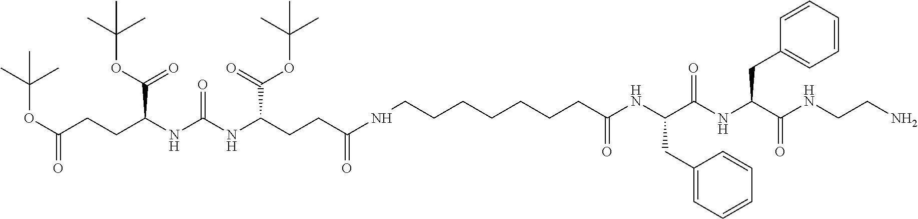 [SMILES: CC(C)(C)OC(=O)CC[C@H](NC(=O)N[C@@H](CCC(=O)NCCCCCCCC(=O)N[C@@H](Cc1ccccc1)C(=O)N[C@@H](Cc1ccccc1)C(=O)NCCN)C(=O)OC(C)(C)C)C(=O)OC(C)(C)C]

To a solution of tri-tert-butyl (9S,12S,26S,30S)-9,12-dibenzyl-3,8,11,14,23,28-hexaoxo-1-phenyl-2-oxa-4,7,10,13,22,27,29-heptaazadotriacontane-26,30,32-tricarboxylate (425 mg, 0.386 mmol) in methanol (3.9 mL) under nitrogen was added Pd—C (41.1 mg, 0.039 mmol). The flask was evacuated and back-filled with a hydrogen gas balloon and stirred at RT for 3 nights. The reaction was evacuated and back-filled with nitrogen gas. The mixture was then filtered through a celite pad, washing with additional MeOH (2×20 mL). The filtrate was concentrated in vacuo to provide the title compound as a white solid (366 mg, 0.379 mmol, 98.1% yield). LC-MS m/z 966.8 (M+H)⁺.

### Intermediate 5: di-tert-butyl (((S)-6-((S)-2-amino-3-(naphthalen-2-yl)propanamido)-1-(tert-butoxy)-1-oxohexan-2-yl)carbamoyl)-L-glutamate

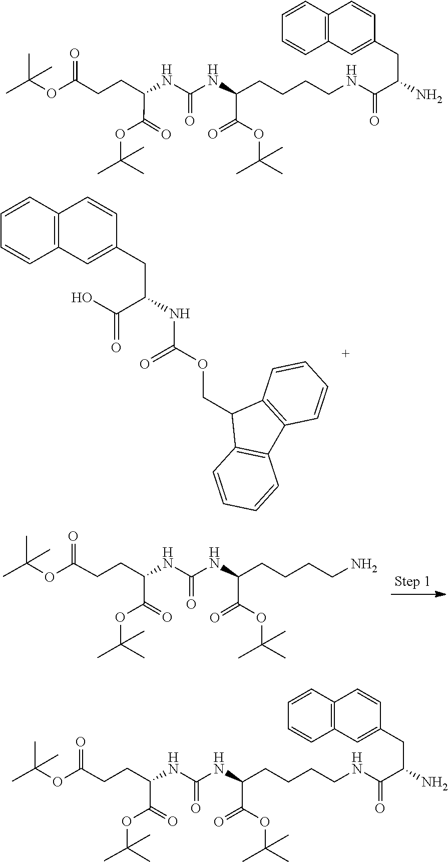

### Step 1: Di-tert-butyl (((S)-6-((S)-2-amino-3-(naphthalen-2-yl)propanamido)-1-(tert-butoxy)-1-oxohexan-2-yl)carbamoyl)-L-glutamate

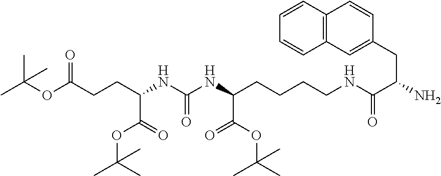 [SMILES: CC(C)(C)OC(=O)CC[C@H](NC(=O)N[C@@H](CCCCNC(=O)[C@@H](N)Cc1ccc2ccccc2c1)C(=O)OC(C)(C)C)C(=O)OC(C)(C)C]

To a solution of (S)-2-((((9H-fluoren-9-yl)methoxy)carbonyl)amino)-3-(naphthalen-2-yl)propanoic acid (269 mg, 0.615 mmol) and HATU (304 mg, 0.800 mmol) in anhydrous N,N-dimethylformamide (6.2 mL) was added DIPEA (215 μl, 1.23 mmol). After 10 minutes, di-tert-butyl (((S)-6-amino-1-(tert-butoxy)-1-oxohexan-2-yl)carbamoyl)-L-glutamate (300 mg, 0.615 mmol) was added and the reaction was stirred at RT for 23 h. Piperidine (122 μl, 1.23 mmol) was added, and the mixture was stirred at RT for 3 h. The reaction was diluted with EtOAc (50 mL), washed with saturated aqueous sodium bicarbonate (2×30 mL) and brine (30 mL), dried over anhydrous sodium sulfate, filtered, and concentrated in vacuo. The resultant residue was purified by ISCO CombiFlash® chromatography eluting with a gradient of 0 to 20% ethyl acetate in hexanes to provide the title compound as a yellow solid (261 mg, 381 mmol, 61.9% yield). LC-MS m/z 685.4 (M+H)⁺.

### Example 1

### (((S)-1-Carboxy-4-(4-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carbonyl)piperazin-1-yl)-4-oxobutyl)carbamoyl)-L-glutamic acid

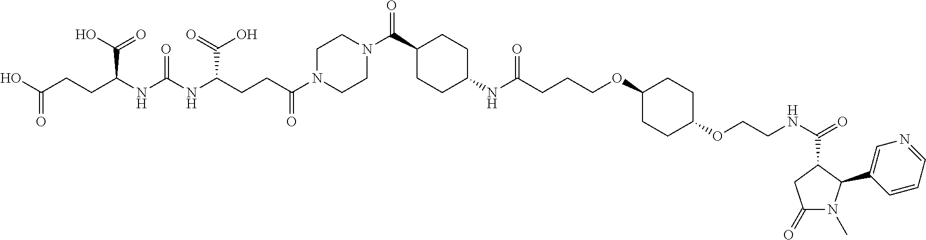 [SMILES: CN1C(=O)C[C@H](C(=O)NCCO[C@H]2CC[C@H](OCCCC(=O)N[C@H]3CC[C@H](C(=O)N4CCN(C(=O)CC[C@H](NC(=O)N[C@@H](CCC(=O)O)C(=O)O)C(=O)O)CC4)CC3)CC2)[C@H]1c1cccnc1]

### Preparation

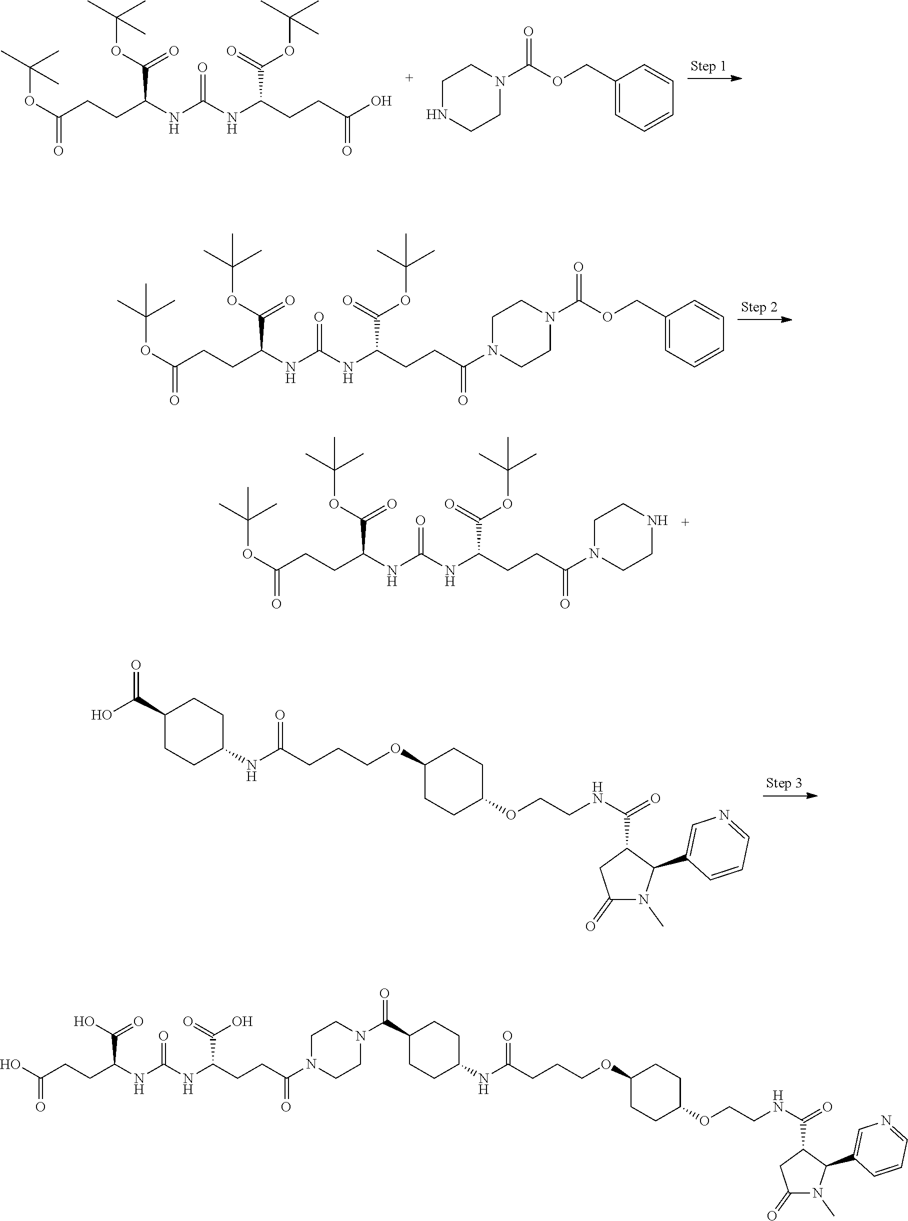

### Step 1: Di-tert-butyl (((S)-5-(4-((benzyloxy)carbonyl)piperazin-1-yl)-1-(tert-butoxy)-1,5-dioxopentan-2-yl)carbamoyl)-L-glutamate

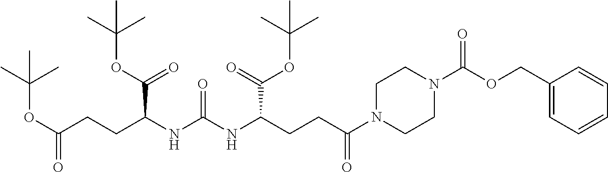 [SMILES: CC(C)(C)OC(=O)CC[C@H](NC(=O)N[C@@H](CCC(=O)N1CCN(C(=O)OCc2ccccc2)CC1)C(=O)OC(C)(C)C)C(=O)OC(C)(C)C]

To a solution of (S)-5-(tert-butoxy)-4-(3-((S)-1,5-di-tert-butoxy-1,5-dioxopentan-2-yl)ureido)-5-oxopentanoic acid (128 mg, 0.262 mmol) and HATU (114 mg, 0.299 mmol) in acetonitrile (1.8 mL) was added DIPEA (0.087 mL, 0.499 mmol). After 10 min, the mixture was added to a vial containing benzyl piperazine-1-carboxylate (55.0 mg, 0.250 mmol). After 1 h, the mixture was purified by ISCO CombiFlash® chromatography eluting with a gradient of 0 to 100% ethyl acetate in hexanes to provide the title compound as a yellow oil (174 mg, 0.269 mmol, >theoretical yield) LC-MS m/z 691.4 (M+H)⁺.

### Step 2: Di-tert-butyl (((S)-1-(tert-butoxy)-1,5-dioxo-5-(piperazin-1-yl)pentan-2-yl)carbamoyl)-L-glutamate

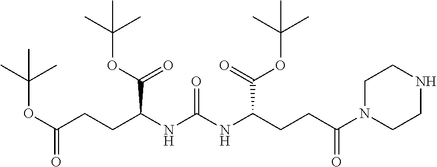 [SMILES: CC(C)(C)OC(=O)CC[C@H](NC(=O)N[C@@H](CCC(=O)N1CCNCC1)C(=O)OC(C)(C)C)C(=O)OC(C)(C)C]

To a solution of di-tert-butyl (((S)-5-(4-((benzyloxy)carbonyl)piperazin-1-yl)-1-(tert-butoxy)-1,5-dioxopentan-2-yl)carbamoyl)-L-glutamate (145 mg, 0.210 mmol) in methanol (4 mL) under an atmosphere of nitrogen was added Pd—C (21.27 mg, 0.200 mmol). The mixture was evacuated and back-filled with a hydrogen gas balloon and allowed to stir at RT for 20 h. The flask was evacuated and back-filled with nitrogen gas. Additional Pd—C (21.27 mg, 0.200 mmol) was added. The mixture was evacuated and back-filled with a hydrogen gas balloon and allowed to stir at RT for 22 h. The flask was evacuated and back-filled with nitrogen gas. The mixture was then filtered through a celite plug, concentrated in vacuo, and dried under high vac to provide the crude title compound as a clear oil that was used without further purification (141 mg, 0.253 mmol, >theoretical yield). LC/MS m/z 557.5 (M+H)⁺.

### Step 3: (((S)-1-Carboxy-4-(4-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexane-1-carbonyl)piperazin-1-yl)-4-oxobutyl)carbamoyl)-L-glutamic acid

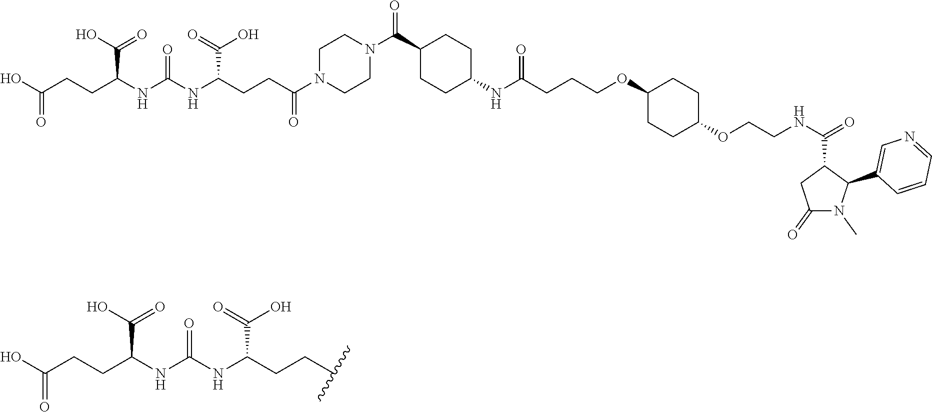 [SMILES: CN1C(=O)C[C@H](C(=O)NCCO[C@H]2CC[C@H](OCCCC(=O)N[C@H]3CC[C@H](C(=O)N4CCN(C(=O)CC[C@H](NC(=O)N[C@@H](CCC(=O)O)C(=O)O)C(=O)O)CC4)CC3)CC2)[C@H]1c1cccnc1 ; *CC[C@H](NC(=O)N[C@@H](CCC(=O)O)C(=O)O)C(=O)O]

To a solution of (1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylic acid (Intermediate 2) (40 mg, 0.070 mmol) in N,N-Dimethylformamide (1.4 mL) was added HATU (34.5 mg, 0.091 mmol) and DIPEA (24.40 μl, 0.140 mmol). Di-tert-butyl (((S)-1-(tert-butoxy)-5-(4-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carbonyl)piperazin-1-yl)-1,5-dioxopentan-2-yl)carbamoyl)-L-glutamate (38.9 mg, 0.70 mmol) was added and the mixture was stirred at RT for 2.5 h. The reaction was diluted with EtOAc (20 mL), washed with saturated aqueous sodium bicarbonate (20 mL) and brine (20 mL), dried over anhydrous sodium sulfate, filtered, and concentrated in vacuo. To a solution of the resultant residue in DCM (1.404 mL) was added TFA (541 μl, 7.02 mmol). The reaction was stirred at RT for 18 h, then concentrated in vacuo. The resultant residue was purified via MDAP (XSelect™ CSH C18 column, 40 mL/min) eluting with a gradient of 5 to 35% acetonitrile in water containing formic acid (0.1%) to provide the title compound as an off-white solid (29.7 mg, 0.0315 mmol, 44.9% yield). LC-MS m/z (M+H)+943.3. ¹H NMR (400 MHZ, METHANOL-d₄) δ ppm 1.19-1.41 (m, 6H) 1.53-1.69 (m, 2H) 1.80-1.86 (m, 4H) 1.89-2.04 (m, 8H) 2.12-2.21 (m, 1H) 2.23-2.31 (m, 3H) 2.40-2.53 (m, 3H) 2.54-2.75 (m, 7H) 2.80-2.92 (m, 1H) 3.02-3.13 (m, 1H) 3.24-3.41 (m, 5H) 3.43-3.58 (m, 6H) 3.59-3.73 (m, 6H) 4.26-4.40 (m, 2H) 7.70-7.79 (m, 1H) 8.03-8.12 (m, 5H) 8.66 (s 1H) 8.70 (br d, J=4.9 Hz, 1H).

The following compounds were or could be prepared with procedures analogous to that described in Example 1:

| | | LC- | |
|---|---|---|---|
| Ex. | Compound | MS m/z | NMR |
| 2 | 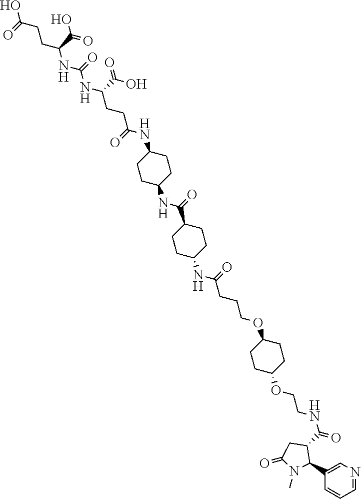 [SMILES: CN1C(=O)C[C@H](C(=O)NCCO[C@H]2CC[C@H](OCCCC(=O)N[C@H]3CC[C@H](C(=O)N[C@H]4CC[C@@H](NC(=O)CC[C@H](NC(=O)N[C@@H](CCC(=O)O)C(=O)O)C(=O)O)CC4)CC3)CC2)[C@H]1c1cccnc1] | 971.7 (M + H)+ | ¹H NMR (400 MHz, METHANOL-d₄) δ ppm 1.18-1.41 (m, 6 H) 1.52- 1.64 (m, 2 H) 1.79- 2.03 (m, 12 H) 2.11- 2.51 (m, 9 H) 2.66-2.75 (m, 4 H) 2.82-2.93 (m, 1 H) 3.00-3.16 (m, 1 H) 3.23-3.41 (m, 11 H) 3.44-3.53 (m, 4 H) 3.58- 3.70 (m, 1 H) 3.73- 3.87 (m, 2 H) 4.23-4.42 (m, 2 H) 4.93-4.99 (m, 2 H) 7.77-7.84 (m, 1 H) 8.05-8.11 (m, 1 H) 8.12- 8.19 (m, 1 H) 8.67- 8.70 (m, 1 H) 8.71-8.76 (m, 1 H). |
| | (((S)-1-Carboxy-4-(((1S,4S)-4-((1R,4S)-4-(4- | | |
| | (((1S,4R)-4-(2-((2S,3S)-1-methyl-5-oxo-2-(pyridin-3- | | |
| | yl)pyrrolidine-3-carboxamido)ethoxy) | | |
| | cyclohexyl)oxy)butanamido)cyclohexane-1- | | |
| | carboxamido)cyclohexyl)amino)-4-oxobutyl) | | |
| | carbamoyl)-L-glutamic acid | | |
| 3 | 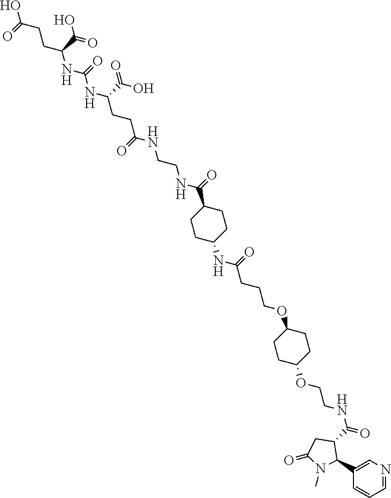 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)N[C@@H](CCC(=O)O)C(=O)O)C(=O)O)CC3)CC2)[C@H]1c1cccnc1] | 917.3 (M + H)+ | ¹H NMR (400 MHz, METHANOL-d₄) δ ppm 1.19-1.35 (m, 6 H) 1.52- 1.67 (m, 2 H) 1.76- 2.03 (m, 13 H) 2.11- 2.39 (m, 7 H) 2.41-2.51 (m, 2 H) 2.65-2.76 (m, 4 H) 2.81-2.94 (m, 1 H) 3.03-3.26 (m, 4 H) 3.26- 3.31 (m, 3 H) 3.35- 3.42 (m, 2 H) 3.44-3.53 (m, 4 H) 3.57-3.71 (m, 1 H) 4.26-4.38 (m, 2 H) 4.94-4.96 (m, 1 H) 7.78- 7.84 (m, 1 H) 8.12- 8.17 (m, 1 H) 8.67-8.70 (m, 1 H) 8.71-8.75 (m, 1 H). |
| | (9S,13S)-1-((1R,4R)-4-(4-(((1S,4R)-4-(2-((2S,3S)-1- | | |
| | Methyl-5-oxo-2-(pyridin-3-yl)pyrrolidine-3- | | |
| | carboxamido)ethoxy)cyclohexyl)oxy)butanamido) | | |
| | cyclohexyl)-1,6,11-trioxo-2,5,10,12- | | |
| | tetraazapentadecane-9,13,15-tricarboxylic acid | | |

### Example 4

### (((S)-1-Carboxy-4-(((1R,4R)-4-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxamido)cyclohexyl)amino)-4-oxobutyl)carbamoyl)-L-glutamic acid

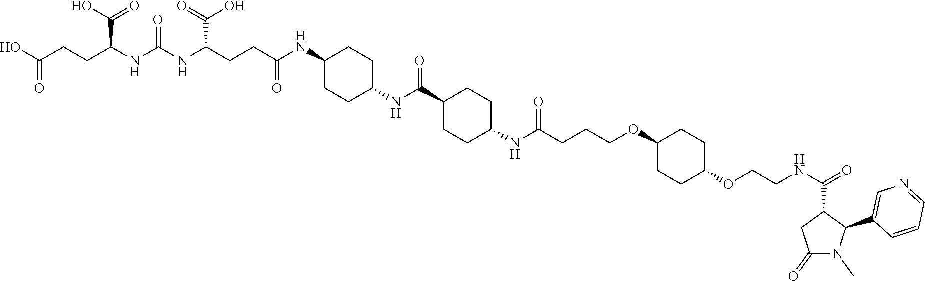 [SMILES: CN1C(=O)C[C@H](C(=O)NCCO[C@H]2CC[C@H](OCCCC(=O)N[C@H]3CC[C@H](C(=O)N[C@H]4CC[C@H](NC(=O)CC[C@H](NC(=O)N[C@@H](CCC(=O)O)C(=O)O)C(=O)O)CC4)CC3)CC2)[C@H]1c1cccnc1]

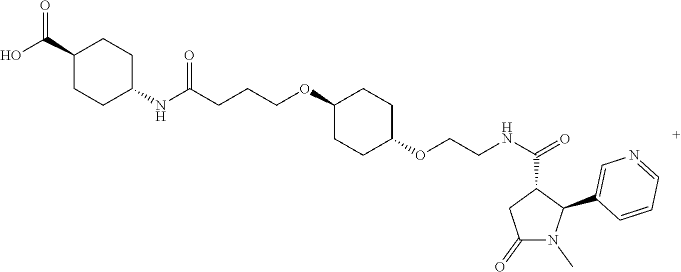 [SMILES: CN1C(=O)C[C@H](C(=O)NCCO[C@H]2CC[C@H](OCCCC(=O)N[C@H]3CC[C@H](C(=O)O)CC3)CC2)[C@H]1c1cccnc1]

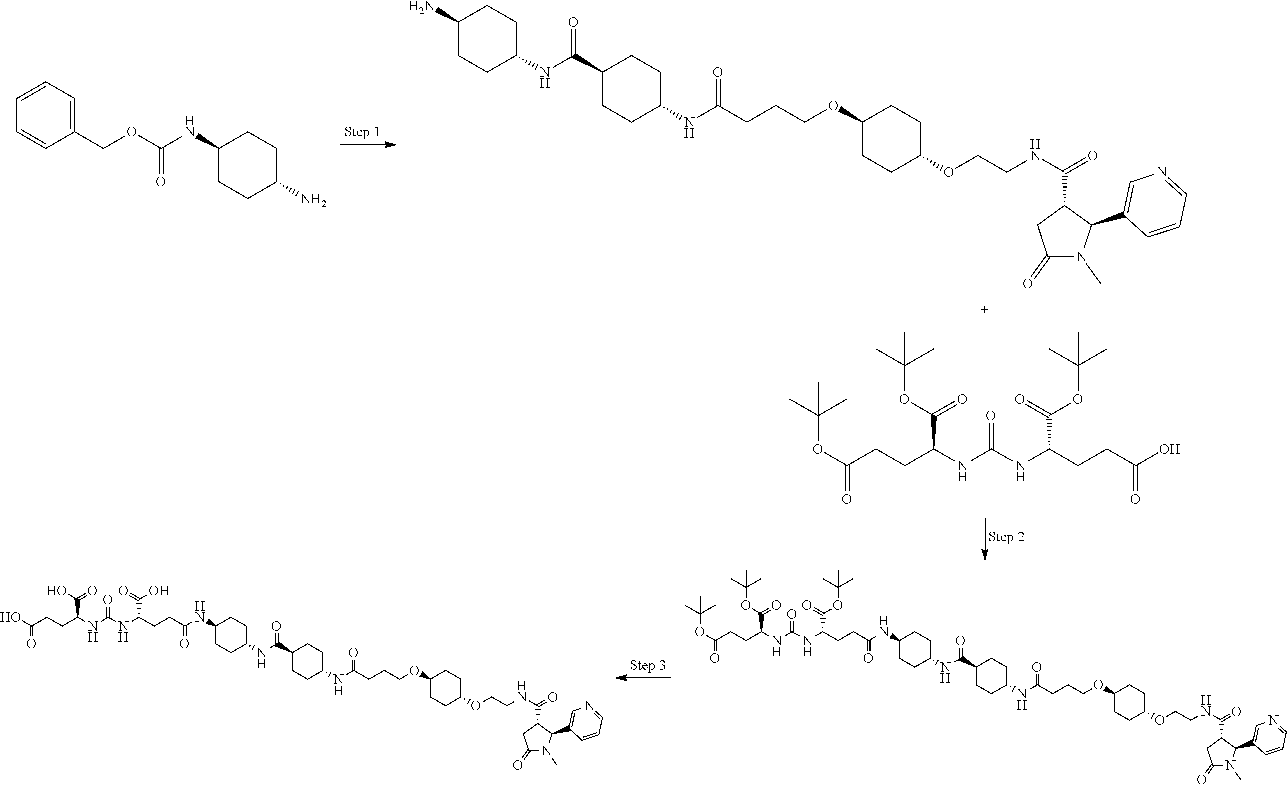

### Step 1: (2S,3S)—N-(2-(((1R,4S)-4-(4-(((1R,4R)-4-(((1R,4R)-4-aminocyclohexyl) carbamoyl)cyclohexyl)amino)-4-oxobutoxy)cyclohexyl)oxy)ethyl)-1-methyl-5-oxo-2-(pyridin-3-yl)pyrrolidine-3-carboxamide

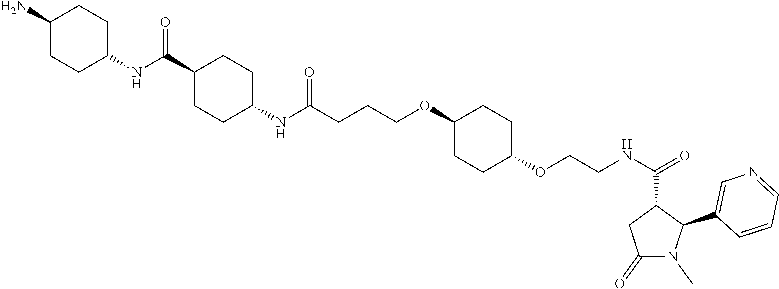

Multi-step reaction: To a solution of benzyl ((1R,4R)-4-aminocyclohexyl)carbamate (81 mg, 0.326 mmol), (1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexane-1-carboxylic acid (Intermediate 2) (178 mg, 0.311 mmol), and HATU (154 mg, 0.404 mmol) in N,N-Dimethylformamide (3.1 mL) was added DIPEA (162 μl, 0.932 mmol). The mixture was stirred at RT for 25 h, then diluted with EtOAc (20 mL) and saturated aqueous sodium bicarbonate (20 mL). The resultant solids were collected via filtration, washed with 10% MeOH in DCM (20 mL), and dried in a 50° C. drying oven for 16 hours to provide a white solid. The solid was suspended in methanol (3.0 mL) and under nitrogen was added Pd/C (31.8 mg, 0.030 mmol). The reaction flask was evacuated and back-filled with a hydrogen balloon and allowed to stir at RT for 5.5 h. The suspension was filtered and concentrated in vacuo to provide the crude title compound as a white solid that was used without further purification (316 mg, 0.472 mmol, >theoretical yield). LCMS m/z 669.5 (M+H)⁺.

### Step 2: Di-tert-butyl (((S)-1-(tert-butoxy)-5-(((1R,4R)-4-((1R,4R)-4-(4-(((1S,4R)-4-(2-((2S,3S)-1-methyl-5-oxo-2-(pyridin-3-yl)pyrrolidine-3-carboxamido)ethoxy) cyclohexyl)oxy)butanamido)cyclohexane-1-carboxamido)cyclohexyl)amino)-1,5-dioxopentan-2-yl)carbamoyl)-L-glutamate

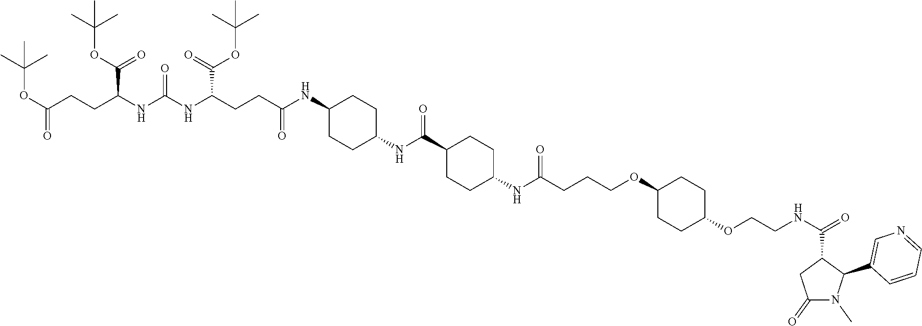 [SMILES: CN1C(=O)C[C@H](C(=O)NCCO[C@H]2CC[C@H](OCCCC(=O)N[C@H]3CC[C@H](C(=O)N[C@H]4CC[C@H](NC(=O)CC[C@H](NC(=O)N[C@@H](CCC(=O)OC(C)(C)C)C(=O)OC(C)(C)C)C(=O)OC(C)(C)C)CC4)CC3)CC2)[C@H]1c1cccnc1]

To a solution of (S)-5-(tert-butoxy)-4-(3-((S)-1,5-di-tert-butoxy-1,5-dioxopentan-2-yl)ureido)-5-oxopentanoic acid (50 mg, 0.102 mmol), (2S,3S)—N-(2-(((1R,4S)-4-(4-(((1R,4R)-4-(((1R,4R)-4-aminocyclohexyl)carbamoyl)cyclohexyl)amino)-4-oxobutoxy)cyclohexyl)oxy)ethyl)-1-methyl-5-oxo-2-(pyridin-3-yl)pyrrolidine-3-carboxamide (68.5 mg, 0.102 mmol), and HATU (50.6 mg, 0.133 mmol) in N,N-Dimethylformamide (3.4 mL) was added DIPEA (50.2 μl, 0.307 mmol). The reaction was stirred at RT for 3 nights, then filtered and purified by MDAP (XSelect™ CSH C18 column, 40 mL/min) eluting with a gradient of 30 to 85% acetonitrile in water containing formic acid (0.1%) to provide the title compound as an off-white solid (14.7 mg, 0.0129 mmol, 12.6% yield). LC-MS m/z (M+H)⁺ 1139.8.

### Step 3: (((S)-1-Carboxy-4-(((1R,4R)-4-((1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy) butanamido)cyclohexane-1-carboxamido)cyclohexyl)amino)-4-oxobutyl)carbamoyl)-L-glutamic acid

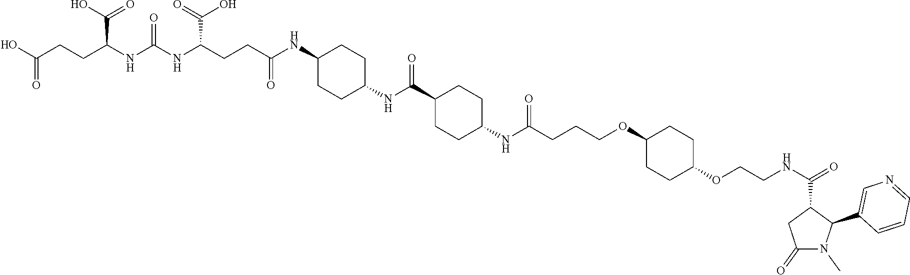 [SMILES: CN1C(=O)C[C@H](C(=O)NCCO[C@H]2CC[C@H](OCCCC(=O)N[C@H]3CC[C@H](C(=O)N[C@H]4CC[C@H](NC(=O)CC[C@H](NC(=O)N[C@@H](CCC(=O)O)C(=O)O)C(=O)O)CC4)CC3)CC2)[C@H]1c1cccnc1]

To a solution of di-tert-butyl (((S)-1-(tert-butoxy)-5-(((1R,4R)-4-((1R,4R)-4-(4-(((1S,4R)-4-(2-((2S,3S)-1-methyl-5-oxo-2-(pyridin-3-yl)pyrrolidine-3-carboxamido)ethoxy) cyclohexyl)oxy)butanamido)cyclohexane-1-carboxamido)cyclohexyl)amino)-1,5-dioxopentan-2-yl)carbamoyl)-L-glutamate (19 mg, 0.017 mmol) in Dichloromethane (1.8 mL) was added TFA (0.064 mL, 0.834 mmol). The reaction was stirred at RT for 26 h. Additional TFA (0.064 mL, 0.834 mmol) was added, and the mixture was stirred at RT for 22 h. The reaction was concentrated in vacuo, and the resultant residue was residue was purified by MDAP (XSelect™ CSH C18 column, 40 mL/min) eluting with a gradient of 5 to 35% acetonitrile in water containing formic acid (0.1%) to provide the title compound as a white solid (8.4 mg, 0.00865 mmol, 51.9% yield). LC-MS m/z (M+H)⁺ 971.7. ¹H NMR (400 MHZ, METHANOL-d₄) δ ppm 1.11-1.40 (m, 7H) 1.50-1.68 (m, 2H) 1.76-2.02 (m, 11H) 2.05-2.34 (m, 5H) 2.35-2.46 (m, 2H) 2.58-2.75 (m, 3H) 2.77-2.91 (m, 1H) 2.98-3.13 (m, 1H) 3.20-3.41 (m, 16H) 3.44-3.54 (m, 3H) 3.56-3.75 (m, 3H) 4.16-4.32 (m, 2H) 4.81-4.86 (m, 1H) 7.50-7.59 (m, 1H) 7.72-7.90 (m, 3H) 8.48-8.54 (m, 1H) 8.56-8.62 (m, 1H).

### Example 5: (7S,10S,24S,28S)-7,10-Dibenzyl-1-((2S,3S)-1-methyl-5-oxo-2-(pyridin-3-yl)pyrrolidin-3-yl)-1,6,9,12,21,26-hexaoxo-2,5,8,11,20,25,27-heptaazatriacontane-24,28,30-tricarboxylic acid

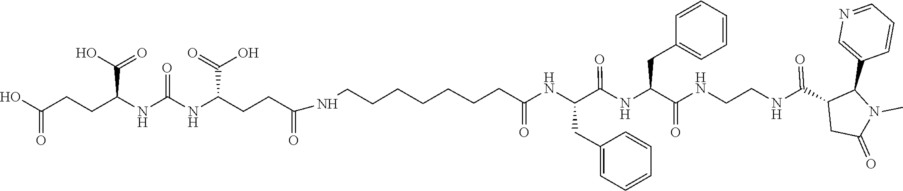 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1]

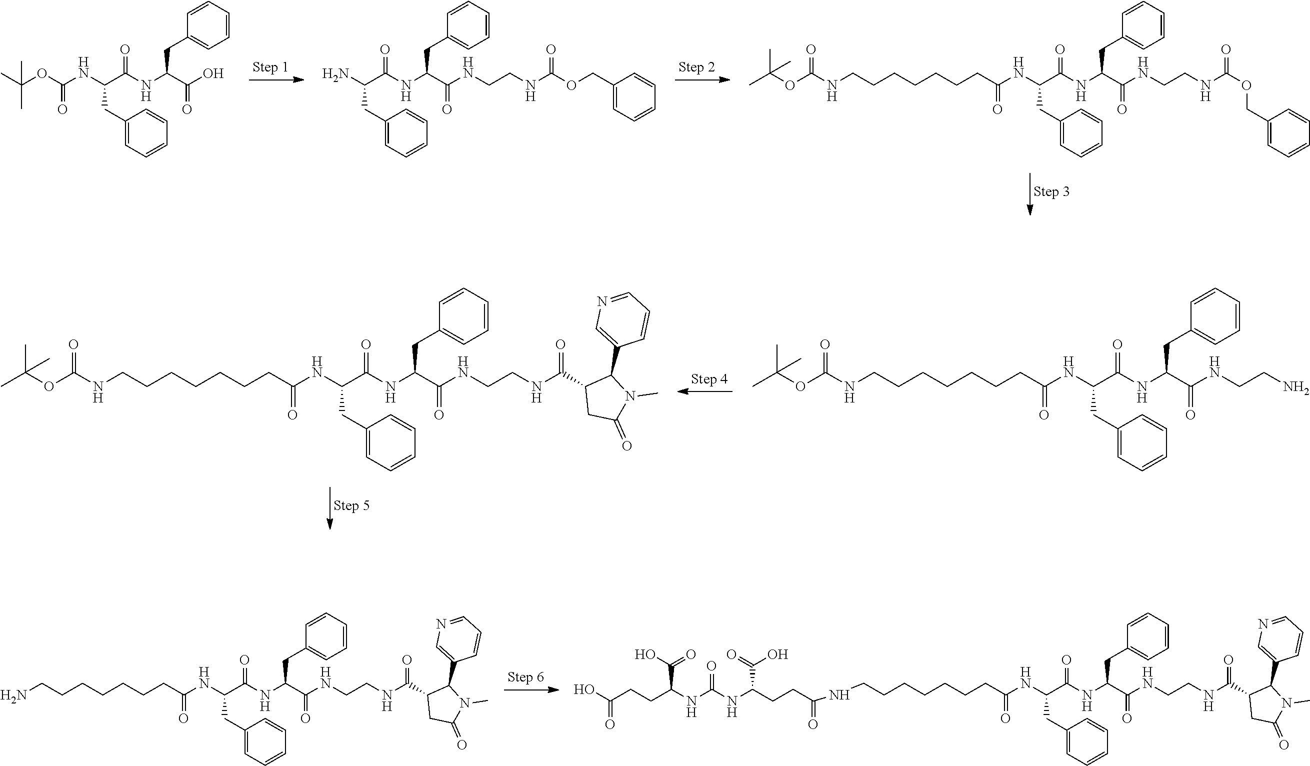

### Step 1: Benzyl (2-((S)-2-((S)-2-amino-3-phenylpropanamido)-3-phenylpropanamido)ethyl)carbamate

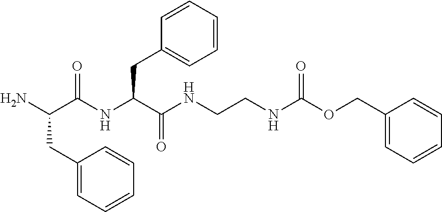 [SMILES: N[C@@H](Cc1ccccc1)C(=O)N[C@@H](Cc1ccccc1)C(=O)NCCNC(=O)OCc1ccccc1]

To a solution of (tert-butoxycarbonyl)-L-phenylalanyl-L-phenylalanine (1.00 g, 2.42 mmol), benzyl (2-aminoethyl)carbamate (0.494 g, 2.55 mmol), and HATU (1.198 g, 3.15 mmol) in anhydrous N,N-dimethylformamide (8.08 mL) was added DIPEA (0.847 ml, 4.85 mmol). The reaction was stirred at RT for 3.5 h, then diluted with EtOAc (100 mL). A beige solid was collected via filtration, washed with water (50 mL) and EtOAc (50 mL), and dried in a vacuum oven at 50° C. for 3 nights. The filtrate was separated and the organic layer was washed with saturated aqueous sodium bicarbonate (2×30 mL) and brine (30 mL), dried over anhydrous sodium sulfate, filtered, and concentrated in vacuo. The resultant residue was combined with the isolated solid, and the mixture was suspended in dichloromethane (12.1 mL). TFA (1.86 mL, 24.1 mmol) was added. The reaction became a clear yellow solution and was stirred at RT for 3 days. Additional TFA (1.86 mL, 24.1 mmol) was added, and the mixture was stirred another 24 h. The reaction was concentrated in vacuo, and the resultant residue was redissolved in 10% MeOH in DCM (100 mL). Saturated aqueous sodium bicarbonate was added to pH=8-10. The phases were separated, and the aqueous layer was extracted with 10% MeOH in DCM (3×50 mL). The combined organics were dried over sodium sulfate, filtered, and concentrated in vacuo. The resultant residue was purified by ISCO CombiFlash® chromatography eluting with a gradient of 0 to 10% methanol in dichloromethane to provide the title compound as a white solid (916.2 mg, 1.875 mmol, 77.7% yield). LCMS m/z 489.3 (M+H)⁺.

### Step 2: Benzyl ((15S,18S)-15,18-dibenzyl-2,2-dimethyl-4,13,16,19-tetraoxo-3-oxa-5,14,17,20-tetraazadocosan-22-yl)carbamate

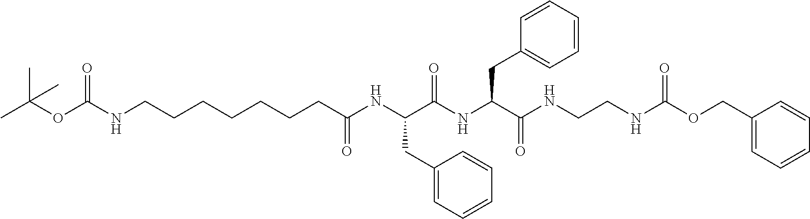 [SMILES: CC(C)(C)OC(=O)NCCCCCCCC(=O)N[C@@H](Cc1ccccc1)C(=O)N[C@@H](Cc1ccccc1)C(=O)NCCNC(=O)OCc1ccccc1]

To a solution of benzyl (2-((S)-2-((S)-2-amino-3-phenylpropanamido)-3-phenylpropanamido)ethyl)carbamate (1 g, 2.047 mmol), 8-((tert-butoxycarbonyl)amino)octanoic acid (0.557 g, 2.149 mmol), and HATU (1.01 g, 2.66 mmol) in DCM (20.5 mL) was added DIPEA (0.715 ml, 4.09 mmol). The reaction was stirred at RT for 1 h. To the gelatinous suspension was added DCM (30 mL). The mixture was stirred vigorously at RT for 4 nights. The mixture was concentrated to remove the organics, and the resultant solid was washed with aqueous sodium bicarbonate (2×20 mL) and water (20 mL) to provide the crude title compound as an orange solid (2.012 g, 2.76 mmol, >theoretical yield). LC-MS m/z 730.3 (M+H)⁺.

### Step 3: tert-Butyl (8-(((S)-1-(((S)-1-((2-aminoethyl)amino)-1-oxo-3-phenylpropan-2-yl)amino)-1-oxo-3-phenylpropan-2-yl)amino)-8-oxooctyl)carbamate

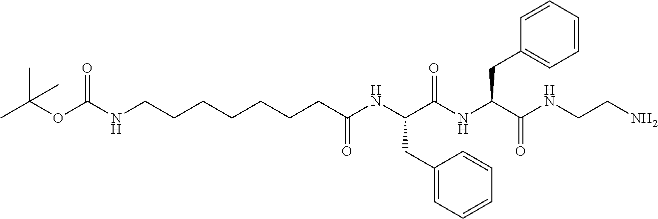 [SMILES: CC(C)(C)OC(=O)NCCCCCCCC(=O)N[C@@H](Cc1ccccc1)C(=O)N[C@@H](Cc1ccccc1)C(=O)NCCN]

To a suspension of benzyl ((15S,18S)-15,18-dibenzyl-2,2-dimethyl-4,13,16,19-tetraoxo-3-oxa-5,14,17,20-tetraazadocosan-22-yl)carbamate (583 mg, 0.799 mmol) in anhydrous methanol (8 mL) under nitrogen gas was added Pd—C (85.0 mg, 0.799 mmol). The reaction was evacuated, back-filled with hydrogen gas (balloon), and stirred at RT for 3 nights. The flask was evacuated and back-filled with nitrogen gas and allowed to sit over the weekend. Then additional methanol (8 mL) and Pd—C (85.0 mg, 0.799 mmol) were added. The flask was evacuated and back-filled with hydrogen gas (balloon), and stirred at RT for 24 hours. The reaction was evacuated and back-filled with nitrogen gas, then filtered through a celite plug, washing with additional methanol (2×30 mL). The combined filtrates were concentrated in vacuo and dried on high vac to provide the crude title compound as an off-white solid that was used without further purification (908 mg, 1.52 mmol, >theoretical yield) LC-MS m/z 596.3 (M+H)⁺.

### Step 4: tert-Butyl ((7S,10S)-7,10-dibenzyl-1-((2S,3S)-1-methyl-5-oxo-2-(pyridin-3-yl)pyrrolidin-3-yl)-1,6,9,12-tetraoxo-2,5,8,11-tetraazanonadecan-19-yl)carbamate

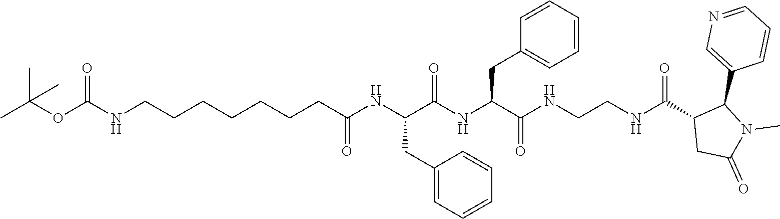 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)OC(C)(C)C)[C@H]1c1cccnc1]

To a solution of (2S,3S)-1-methyl-5-oxo-2-(pyridin-3-yl)pyrrolidine-3-carboxylic acid (Intermediate 1) (100 mg, 0.454 mmol) and HATU (259 mg, 0.681 mmol) in anhydrous N,N-dimethylformamide (4.5 mL) was added DIPEA (238 μl, 1.36 mmol). After 10 minutes, tert-butyl (8-(((S)-1-(((S)-1-((2-aminoethyl)amino)-1-oxo-3-phenylpropan-2-yl)amino)-1-oxo-3-phenylpropan-2-yl)amino)-8-oxooctyl)carbamate (˜50% pure, 542 mg, 0.454 mmol) was added and the mixture was stirred at RT for 17 h. The reaction was diluted with EtOAc (50 mL), washed with saturated aqueous sodium bicarbonate (2×30 mL) and brine (30 mL), dried over anhydrous sodium sulfate, filtered, and concentrated in vacuo. The resultant residue was purified by ISCO CombiFlash® chromatography eluting with a gradient of 0 to 15% methanol in dichloromethane to provide the title compound as a yellow solid (285.5 mg, 0.358 mmol, 78.9% yield). LC-MS m/z 798.5 (M+H)⁺.

### Step 5: (2S,3S)—N-(2-((S)-2-((S)-2-(8-Aminooctanamido)-3-phenyl propanamido)-3-phenylpropanamido)ethyl)-1-methyl-5-oxo-2-(pyridin-3-yl)pyrrolidine-3-carboxamide

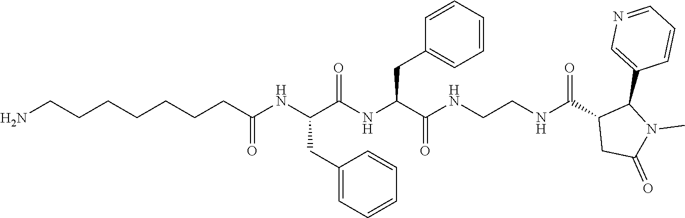 [SMILES: CN1C(=O)C[C@H](C(=O)NCCNC(=O)[C@H](Cc2ccccc2)NC(=O)[C@H](Cc2ccccc2)NC(=O)CCCCCCCN)[C@H]1c1cccnc1]

To a solution of tert-butyl ((7S,10S)-7,10-dibenzyl-1-((2S,3S)-1-methyl-5-oxo-2-(pyridin-3-yl)pyrrolidin-3-yl)-1,6,9,12-tetraoxo-2,5,8,11-tetraazanonadecan-19-yl)carbamate (248 mg, 0.311 mmol) in Dichloromethane (3.1 mL) was added TFA (1.20 mL, 15.5 mmol). The reaction was stirred at RT for 3 hours, then concentrated in vacuo to provide the title compound (assumed theoretical yield) that was used without further purification. LC-MS m/z 698.4 (M+H)⁺.

### Step 6: (7S,10S,24S,28S)-7,10-dibenzyl-1-((2S,3S)-1-methyl-5-oxo-2-(pyridin-3-yl)pyrrolidin-3-yl)-1,6,9,12,21,26-hexaoxo-2,5,8,11,20,25,27-heptaazatriacontane-24,28,30-tricarboxylic acid

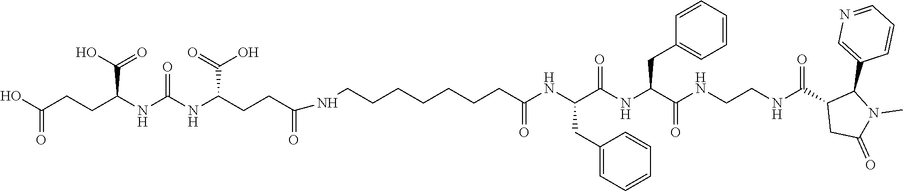 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1]

To a solution of (S)-5-(tert-butoxy)-4-(3-((S)-1,5-di-tert-butoxy-1,5-dioxopentan-2-yl)ureido)-5-oxopentanoic acid (70.0 mg, 0.143 mmol) and HATU (70.8 mg, 0.186 mmol) in N,N-dimethylformamide (1.8 mL) was added DIPEA (0.050 mL, 0.287 mmol). After 10 minutes, a solution of (2S,3S)—N-(2-((S)-2-((S)-2-(8-aminooctanamido)-3-phenylpropanamido)-3-phenyl propanamido)ethyl)-1-methyl-5-oxo-2-(pyridin-3-yl)pyrrolidine-3-carboxamide (100 mg, 0.143 mmol) and DIPEA (0.050 mL, 0.287 mmol) in N,N-Dimethylformamide (1.8 mL) was added. After 10 min, additional DIPEA (0.050 mL, 0.287 mmol) was added, and the mixture was stirred at RT for 3 nights. The reaction was diluted with EtOAc (30 mL) and washed with saturated aqueous sodium bicarbonate (2×20 mL) and brine (20 mL), dried over anhydrous sodium sulfate, filtered, and concentrated in vacuo. The resultant residue was redissolved in DCM (5 mL) and TFA (0.552 mL, 7.16 mmol) and stirred at RT for 24 h. Additional TFA (0.552 mL, 7.16 mmol) was added. The mixture was stirred at RT for 24 h, then concentrated in vacuo. The resultant residue was purified by MDAP (XSelect™ CSH C₁₈ column, 40 mL/min) eluting with a gradient of 30 to 55% acetonitrile in water containing formic acid (0.1%) to provide the title compound as a off-white solid (38.6 mg, 0.0386 mmol, 26.9% yield). LC-MS m/z (M+H)⁺ 1000.6. ¹H NMR (400 MHZ, METHANOL-d₄) δ ppm 1.10-1.20 (m, 2H) 1.21-1.35 (m, 4H) 1.37-1.53 (m, 4H) 1.83-2.01 (m, 2H) 2.05-2.17 (m, 4H) 2.19-2.29 (m, 1H) 2.31 (br dd, J=7.1, 3.2 Hz, 2H) 2.65-2.67 (m, 3H) 2.67-2.69 (m, 1H) 2.70-2.78 (m, 1H) 2.78-2.97 (m, 3H) 2.99-3.08 (m, 2H) 3.08-3.20 (m, 4H) 3.20-3.28 (m, 2H) 3.28-3.32 (m, 1H) 4.10-4.22 (m, 2H) 4.46-4.60 (m, 2H) 4.81-4.89 (m, 1H) 7.06-7.37 (m, 11H) 7.51 (dd, J=7.8, 4.9 Hz, 1H) 7.81 (dt, J=7.8, 2.0 Hz, 1H) 8.43-8.65 (m, 2H).

### Example 6

### (7S,10S,24S,28S)-7,10-dibenzyl-1-((1R,4S)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexyl)-1,6,9,12,21,26-hexaoxo-2,5,8,11,20,25,27-heptaazatriacontane-24,28,30-tricarboxylic acid

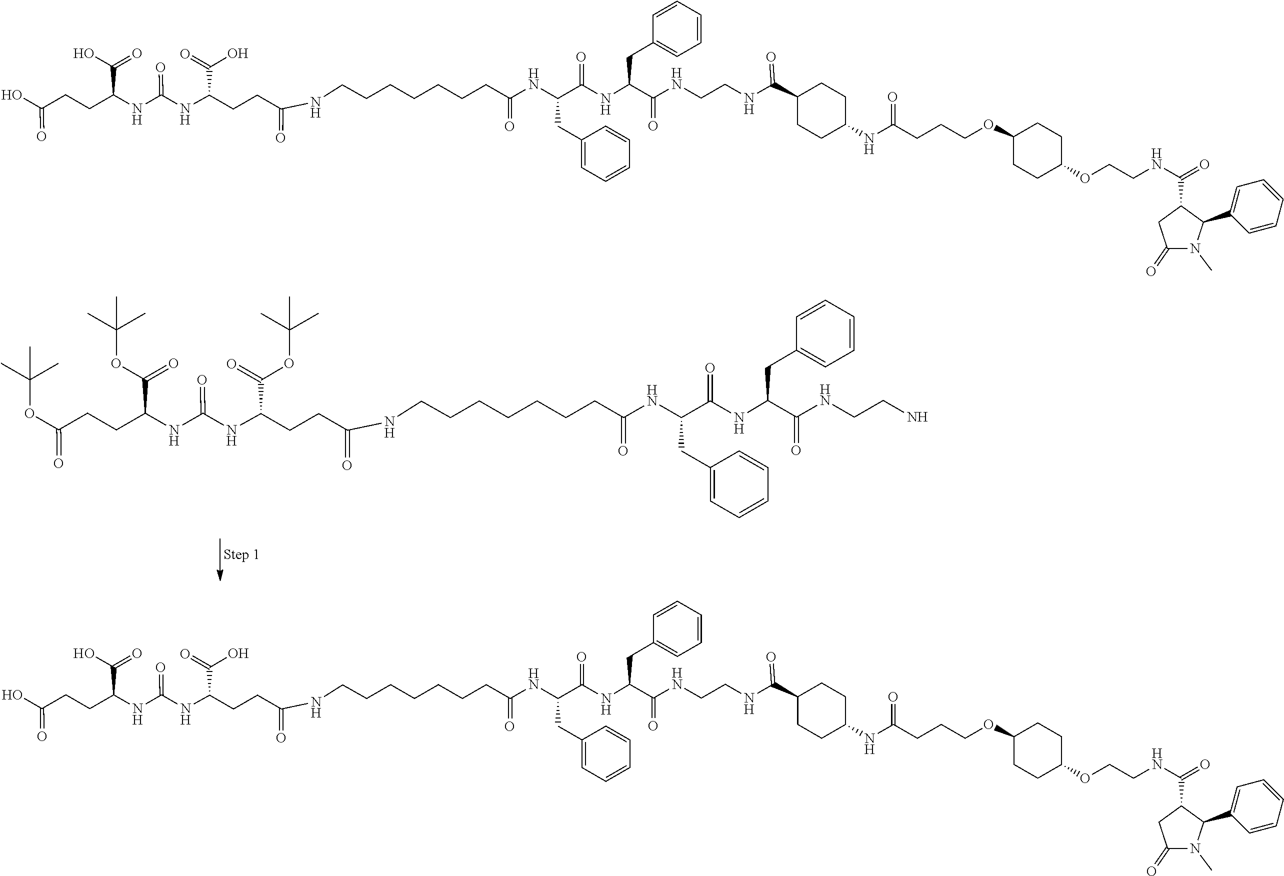

### Step 1: Tri-tert-butyl (7S,10S,24S,28S)-7,10-dibenzyl-1-((1R,4S)-4-(4-((4-(2-((2S,3S)-1-methyl-5-oxo-2-(pyridin-3-yl)pyrrolidine-3-carboxamido)ethoxy)cyclohexyl)oxy) butanamido)cyclohexyl)-1,6,9,12,21,26-hexaoxo-2,5,8,11,20,25,27-heptaazatriacontane-24,28,30-tricarboxylate

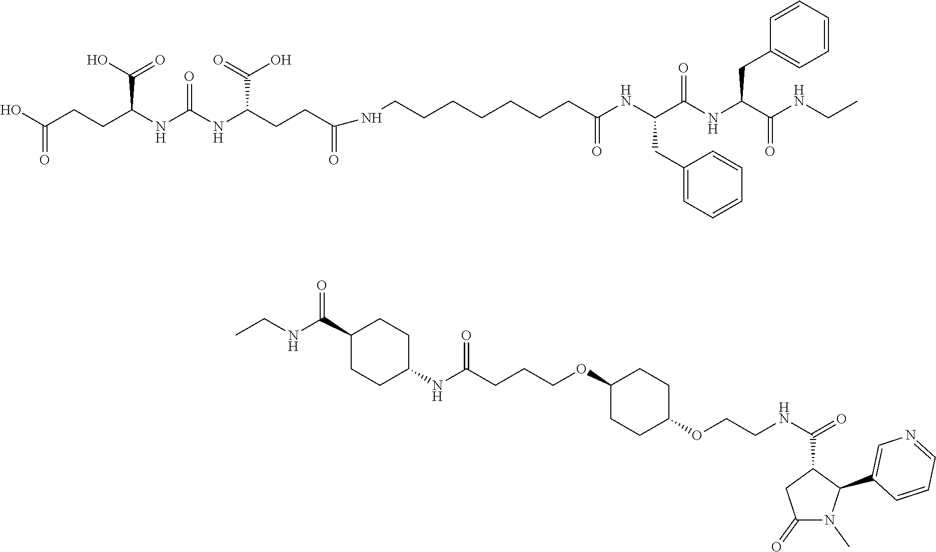

Multi-step reaction: To a solution of tri-tert-butyl (5S,8S,22S,26S)-1-amino-5,8-dibenzyl-4,7,10,19,24-pentaoxo-3,6,9,18,23,25-hexaazaoctacosane-22,26,28-tricarboxylate (Intermediate 4) (79 mg, 0.082 mmol) and (1R,4R)-4-(4-(((1S,4R)-4-(2-((2,3S)-1-methyl-5-oxo-2-(pyridin-3-yl)pyrrolidine-3-carboxamido)ethoxy)cyclohexyl)oxy)butanamido)cyclohexane-1-carboxylic acid hydrochloride (Intermediate 2) (50 mg, 0.082 mmol) in N,N-Dimethylformamide (1.6 mL) was added HATU (40.6 mg, 0.107 mmol) and DIPEA (43.0 μl, 0.246 mmol). The reaction was stirred at RT for 20 h. TFA (63.2 μl, 0.821 mmol) was added, and the mixture was stirred at RT for 4 h. Additional DCM (2 mL) and TFA (63.2 μl, 0.821 mmol) were added, and the reaction was stirred at RT for 21 h. Additional DCM (2 mL) and TFA (0.5 mL) was added, and the gelatinous mixture was stirred at RT another 24 h. Additional TFA (1 mL) was added, and after 3 hours, the mixture was concentrated in vacuo. The resultant residue was resuspended in 4N HCl in dioxane (0.20 mL, 0.82 mmol), and the mixture was stirred at RT for 3 nights. Additional 4N HCl in dioxane (0.5 mL, 2.0 mmol) was added, and the mixture was stirred at RT for 24 h. The mixture was concentrated in vacuo and the resultant residue was redissolved in 4N HCl in dioxane (1.0 mL, 4.0 mmol), and the mixture was stirred at RT for 18 h. Additional 4N HCl in dioxane (3.0 mL, 12.0 mmol) was added, and the mixture was stirred at RT for 7 h. Additional 4N HCl in dioxane (2.0 mL, 8.0 mmol) was added, and the mixture was stirred at RT for 19 h. The reaction was concentrated in vacuo, and the resultant residue was purified twice by MDAP (XSelect™ CSH C18 column, 40 mL/min) eluting with a gradient of 15 to 55% acetonitrile in water containing formic acid (0.1%) to provide the title compound as a white solid (5.8 mg, 0.0043 mmol, 5.22% yield). LC-MS m/2z (M+2H/2)⁺⁺ 677.2. ¹H NMR (400 MHZ, METHANOL-d₄) δ ppm 1.05-1.37 (m, 14H) 1.41-1.63 (m, 5H) 1.75-2.02 (m, 12H) 2.07-2.18 (m, 4H) 2.20-2.34 (m, 4H) 2.47 (s, 2H) 2.65-2.67 (m, 3H) 2.76-2.88 (m, 2H) 2.91-3.00 (m, 1H) 3.03-3.13 (m, 4H) 3.13-3.20 (m, 4H) 3.21-3.28 (m, 7H) 3.43-3.53 (m, 4H) 3.59-3.72 (m, 1H) 4.22-4.38 (m, 2H) 4.47-4.65 (m, 2H) 7.14-7.38 (m, 10H) 7.54 (dd, J=7.82, 4.89 Hz, 1H) 7.75-7.86 (m, 1H) 8.51 (s, 1H) 8.57-8.67 (m, 1H).

The following compounds were or could be prepared with procedures analogous to that described in Example 6:

| | | LC- | |
|---|---|---|---|
| Ex. | Compound | MS m/z | NMR |
| 7 | 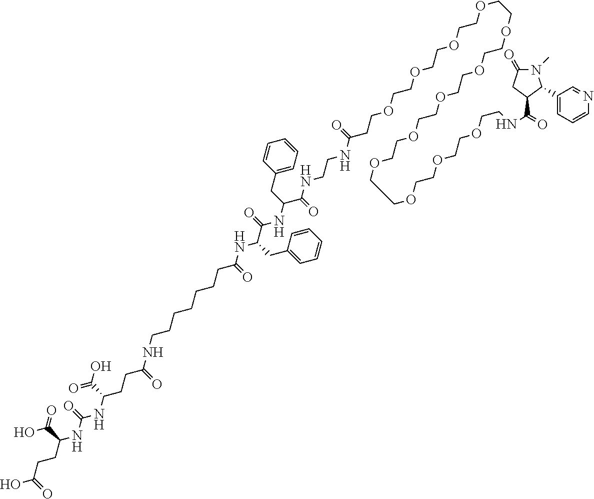 [SMILES: CN1C(=O)C[C@H](C(=O)NCCOCCOCCOCCOCCOCCOCCOCCOCCOCCOCCOCCOCCC(=O)NCCNC(=O)C(Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] | LCMS m/z (M + 2H)⁺⁺ 800.78. | ¹H NMR (400 MHz, METHANOL-d₄) δ ppm 1.11-1.21 (m, 2 H) 1.23- 1.37 (m, 5 H) 1.41- 1.55 (m, 4 H) 1.83-1.99 (m, 2 H) 2.07-2.24 (m, 4 H) 2.26-2.38 (m, 2 H) 2.41-2.52 (m, 3 H) 2.65- 2.71 (m, 13 H) 2.73- 2.93 (m, 3 H) 2.95-3.04 (m, 5 H) 3.06-3.07 (m, 1 H) 3.06-3.21 (m, 4 H) 3.22-3.28 (m, 2 H) 3.37- 3.45 (m, 2 H) 3.46- 3.56 (m, 2 H) 3.56-3.69 (m, 28 H) 3.70-3.82 (m, 3 H) 4.26-4.39 (m, 2 H) 4.42-4.57 (m, 2 H) 4.59- 4.67 (m, 1 H) 4.72- 4.79 (m, 1 H) 7.13-7.38 (m, 10 H) 7.52-7.61 (m, 1 H) 7.80-7.89 (m, 1 H) 8.08-8.18 (m, 1 H) 8.51- 8.56 (m, 1 H) 8.57- 8.64 (m, 1 H). |
| | (47S,50S,64S,68S)-47,50-Dibenzyl-1-((2S,3S)-1- | | |
| | methyl-5-oxo-2-(pyridin-3-yl)pyrrolidin-3-yl)- | | |
| | 1,41,46,49,52,61,66-heptaoxo-5,8,11,14,17, | | |
| | 20,23,26,29,32,35,38-dodecaoxa-2,42,45,48,51, | | |
| | 60,65,67-octaazaheptacontane-64,68,70- | | |
| | tricarboxylic acid | | |

### Example 8

### (((S)-1-Carboxy-5-((S)-2-((1R,4S)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxamido)-3-(naphthalen-2-yl)propanamido)pentyl)carbamoyl)-L-glutamic acid

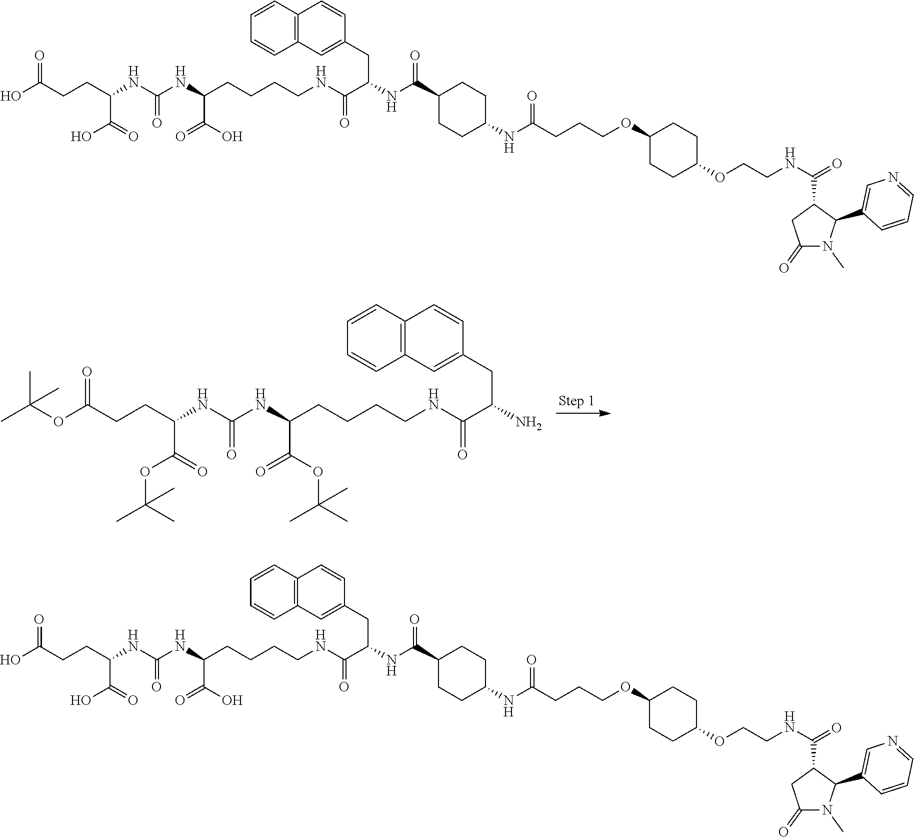 [SMILES: CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1 ; CC(C)(C)OC(=O)CC[C@H](NC(=O)N[C@@H](CCCCNC(=O)[C@@H](N)Cc1ccc2ccccc2c1)C(=O)OC(C)(C)C)C(=O)OC(C)(C)C ; CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1]

### Example 8: (((S)-1-Carboxy-5-((S)-2-((1R,4S)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido) cyclohexane-1-carboxamido)-3-(naphthalen-2-yl)propanamido)pentyl)carbamoyl)-L-glutamic acid

To a solution of (1R,4R)-4-(4-(((1S,4R)-4-(2-((2S,3S)-1-methyl-5-oxo-2-(pyridin-3-yl)pyrrolidine-3-carboxamido)ethoxy)cyclohexyl)oxy)butanamido)cyclohexane-1-carboxylic acid hydrochloride (Intermediate 2) (53.4 mg, 0.088 mmol), di-tert-butyl (((S)-6-((S)-2-amino-3-(naphthalen-2-yl)propanamido)-1-(tert-butoxy)-1-oxohexan-2-yl)carbamoyl)-L-glutamate (Intermediate 5) (60 mg, 0.088 mmol), and HATU (43.3 mg, 0.114 mmol) in N,N-Dimethylformamide (876 μl) was added DIPEA (45.9 μl, 0.263 mmol). The mixture was stirred at RT for 2 h. The reaction was diluted with EtOAc (50 mL), washed with saturated sodium bicarbonate (2×30 mL) and brine (30 mL), dried over anhydrous sodium sulfate, filtered, and concentrated in vacuo. To solution of resultant residue in DCM (5 mL) was added TFA (337 μl, 4.38 mmol). The reaction was stirred at RT for 3 nights. Additional TFA (337 μl, 4.38 mmol) was added. The reaction was stirred at RT for 27 h, then concentrated in vacuo. The resultant residue was purified by MDAP (XSelect™ CSH C18 column, 40 mL/min) eluting with a gradient of 15 to 55% acetonitrile in water containing formic acid (0.1%) to provide the title compound as an off-white solid (31.8 mg, 0.0297 mmol, 33.9% yield). LCMS m/z (M/2+H)⁺ 536.4. ¹H NMR (400 MHz, METHANOL-d₄) δ ppm 1.11-1.42 (m, 10H) 1.43-1.74 (m, 4H) 1.77-1.86 (m, 4H) 1.87-2.01 (m, 6H) 2.10-2.19 (m, 2H) 2.20-2.25 (m, 2H) 2.39-2.47 (m, 2H) 2.64-2.67 (m, 3H) 2.68-2.75 (m, 1H) 2.79-2.89 (m, 1H) 3.03-3.18 (m, 4H) 3.22-3.31 (m, 4H) 3.32-3.35 (m, 1H) 3.35-3.43 (m, 1H) 3.43-3.53 (m, 4H) 3.53-3.62 (m, 1H) 4.20 (dd, J=8.3, 4.9 Hz, 1H) 4.69 (dd, J=8.3, 6.9 Hz, 1H) 4.84 (d, J=6.8, 1H) 7.41 (dd, J=8.3, 1.5 Hz, 1H) 7.43-7.50 (m, 2H) 7.54 (dd, J=7.8, 4.9 Hz, 1H) 7.70 (s, 1H) 7.76-7.86 (m, 4H) 7.99 (br t, J=5.4 Hz, 1H) 852 (d, J=2.0 Hz, 1H) 8.59 (dd, J=4.9, 1.5 Hz, 1H).

The following compound was or could be prepared with procedures analogous to that described in Example 8:

| | | LC- | |
|---|---|---|---|
| Ex. | Compound | MS m/z | NMR |
| 9 | 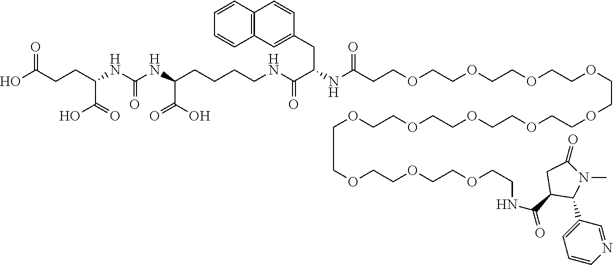 [SMILES: CN1C(=O)C[C@H](C(=O)NCCOCCOCCOCCOCCOCCOCCOCCOCCOCCOCCOCCOCCC(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1] | 660.0 (M + 2H)⁺⁺ | ¹H NMR (400 MHz, METHANOL-d₄) δ ppm 1.24-1.47 (m, 4 H) 1.52- 1.64 (m, 1 H) 1.66- 1.79 (m, 1 H) 1.85-1.97 (m, 1 H) 2.10-2.21 (m, 1 H) 2.33-2.51 (m, 4 H) 2.62-2.76 (m, 4 H) 2.80- 2.91 (m, 1 H) 3.00- 3.20 (m, 4 H) 3.25-3.31 (m, 1 H) 3.35-3.46 (m, 2 H) 3.46-3.54 (m, 6 H) 3.54-3.67 (m, 45 H) 3.68-3.83 (m, 1 H) 4.18- 4.24 (m, 1 H) 4.30- 4.35 (m, 1 H) 4.67-4.74 |
| | (((S)-1-Carboxy-5-((S)-2-(1-((2S,3S)-1- | | (m, 1 H) 4.81-4.86 (m, |
| | methyl-5-oxo-2-(pyridin-3-yl)pyrrolidin-3-yl)-1-oxo- | | 1 H) 7.39-7.50 (m, 3 H) |
| | 5,8,11,14,17,20,23,26,29,32,35,38-dodecaoxa-2- | | 7.51-7.60 (m, 1 H) 7.70- |
| | azahentetracontan-41-amido)-3-(naphthalen-2- | | 7.77 (m, 1 H) 7.79- |
| | yl)propanamido)pentyl)carbamoyl)-L-glutamic acid | | 7.87 (m, 4 H) 7.91-7.99 |
| | | | (m, 1 H) 8.10-8.19 (m, |
| | | | 1 H) 8.50-8.56 (m, 1 H) |
| | | | 8.56-8.62 (m, 1 H). |
| 10 | 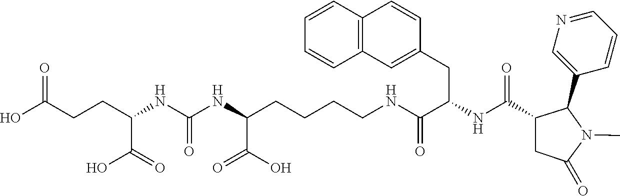 [SMILES: CN1C(=O)C[C@H](C(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1] | 719.33 (M + H)⁺ | ¹H NMR (400 MHz, METHANOL-d₄) δ ppm 1.26-1.47 (m, 4 H) 1.53-1.65 (m, 1 H) 1.66- 1.80 (m, 1 H) 1.84- 1.97 (m, 1 H) 2.09-2.23 (m, 1 H) 2.35-2.50 (m, 3 H) 2.59-2.66 (m, 3 H) 2.67-2.82 (m, 1 H) 3.00- 3.13 (m, 3 H) 3.16- 3.28 (m, 2 H) 4.17-4.27 (m, 1 H) 4.28-4.37 (m, |
| | (((S)-1-Carboxy-5-((S)-2-((2S,3S)-1-methyl-5-oxo- | | 1 H) 4.63-4.78 (m, 1 H) |
| | 2-(pyridin-3-yl)pyrrolidine-3-carboxamido)-3- | | 4.86-4.90 (m, 1 H) 7.37- |
| | (naphthalen-2-yl)propanamido)pentyl) | | 7.42 (m, 1 H) 7.43- |
| | carbamoyl)-L-glutamic acid | | 7.53 (m, 2 H) 7.66-7.70 |
| | | | (m, 1 H) 7.73-7.87 (m, |
| | | | 4 H) 8.03-8.08 (m, 1 H) |
| | | | 8.31-8.37 (m, 1 H) 8.61- |
| | | | 8.65 (m, 1 H) 8.67- |
| | | | 8.71 (m, 1 H) |

### BIOLOGICAL ASSAYS

Example Compounds 1-10 which are compounds of Formulae (I) and (II) having a PSMA binding moiety were tested in various biological assays as described in more detail below.

### Example 11: Antibody Dependent Cellular Cytotoxicity Reporter Assay

An antibody dependent cellular cytotoxicity (ADCC) reporter assay was conducted using the following four assay components: (i) ARM compound of Formula (I) targeting PSMA (concentrations ranging from 1 pM to 10 μM); (ii) anti-cotinine antibody having a heavy chain sequence of SEQ ID NO: 11 and a light chain sequence of SEQ ID NO: 12 (rabbit variable region with human IgG1 Fc domain containing DE mutation (S239D/I332E)) (concentrations ranging from 0.01 μg/mL-200 μg/mL); (iii) Target cells: LNCaP cells (typically 1000-20,000 cells per well); and (iv) Reporter cells—Jurkat cells engineered to express FcγRIIIa with a reporter gene luciferase under the control of the NFAT promoter (typically 3000-75,000 cells per well). Reagents were combined in final volume of 20 μL in 384—well tissue culture treated plate. All four assay components were incubated together for about 12-18 hours. Thereafter, BioGlo Detection reagent (Promega) was added to the wells to lyse the cells and provide a substrate for the luciferase reporter protein.

Luminescence signal was measured on a microplate reader capable of measuring luminescence and signal: background was calculated by dividing the signal of a test well by the signal obtained when no compound of formula (I) was included in the assay. EC50 calculations were done using Graphpad Prism Software, specifically a nonlinear regression curve fit (Y=Bottom+(Top−Bottom)/(1+10\hat{ }((Log EC50−X)*HillSlope))).

ARMs compounds of Formula (I) were tested for ADCC activity in the above assay in one or more experimental runs and the results are shown in Table 3 below. Potency of the compounds of Formula (I) is reported as a pEC50 values. The pEC50 value is the negative log of the EC50 value, wherein the EC50 value is half maximal effective concentration measured in molar (M). For compounds tested in more than one experimental run, the pEC50 value is reported as an average.

TABLE 3
Results for ADCC Reporter Assay (Example 11)

| Compound Example No. | ADCC Reporter Assay pEC50 |
|---|---|
| 1 | 7.3 |
| 2 | 7.3 |
| 3 | 6.9 |
| 4 | 7.3 |
| 5 | 7.4 |
| 6 | 7.4 |
| 7 | 6.9 |
| 8 | 8.65 |
| 9 | 8.05 |
| 10 | Inactive¹ |

¹inactive = pEC50 < 5

### Example 12: PK Analysis

Mice (C57BL6) were dosed intravenously with a PBS solution containing a compound of Formula (I) of Example 8 and Example 9. Peripheral blood from IV dosed mice was analyzed to determine PK properties of the ARM compounds of Formula (I).

Formulations preparation: On the day of experiment, stock solution of the compound of formula (I) was removed from storage at −20° C. and thawed at room temperature. Anti-cotinine antibody having a heavy chain sequence of SEQ ID NO: 13 and a light chain sequence of SEQ ID NO: 14 (rabbit variable region sequence with mouse IgG2a Fc domain), if required was removed from storage at −80° C. and thawed at room temperature. Antibody vials were immediately transferred into wet ice after thawing. Compounds of formula (I) were further diluted in DMSO as per experimental requirements.

Formulation composition: The formulation composition was Saline: DMSO: PBS. Saline was added based on the quantity required and then stock solution of the compound of formula (I) prepared in DMSO, followed by addition of antibody in PBS. Formulations were incubated at room temperature for 30 minutes before administration to the mouse. DMSO was used at 1 to 2% (v/v) in the final formulation.

Administration to Animal: Solution formulation of antibody and compound of formula (I) was injected (bolus injection) to the restrained mouse in the right/left lateral tail vein.

Collection of Blood for PK: Blood was collected at various time points, typically ranging from 0.033 hrs to 72 hours following administration (50 μL/time point) through retro-orbital bleeding under mild isoflurane anesthesia.

Terminal bleeding at end of experiment (72 hr): Approximately 250 μL of blood in K₂EDTA tube and approximately 250 μL of blood in SST (serum separation tube) was collected from each mouse through retro-orbital bleeding under deep isoflurane anesthesia. After bleeding, each mouse was sacrificed by cervical dislocation. The blood distribution at termination was determined as follows: 50 μL of K₂EDTA blood was transferred to another tube for PK

Blood drug concentration analysis: Drug concentration in blood samples was determined by an LC-MS/MS-based bioanalytical method developed at Syngene. Samples were analyzed on Q-Trap, API-5500 LC-MS/MS system coupled with Exion UHPLC system from SCIEX, USA operated in multiple reaction monitoring mode employing electrospray ionization technique in positive polarity. Analyte and internal standard peaks were resolved on Synergi Polar, 75×2.0 mm, 4μ column using mobile phase 10 mM Ammonium acetate in Milli-Q water as phase A and 0.1% Formic acid in acetonitrile as Phase B. Gradient elution was performed with initial composition 95% Phase A at 0.0 min, holding it for 0.2 minutes, ramping to 5% by 1.0 minute, keeping the same for next 0.5 minutes and coming back to 95% by 1.6 minutes. The total run time was 2 minutes.

Working dilutions for calibration curve and quality control standards were prepared by serially diluting 20 mg/mL stock solution with DMSO. Spiked concentrations for calibration curve in the whole blood ranged from 1 ng/mL to 1000 ng/ml. The working solution of internal standard (Verapamil, 25 ng/ml) was prepared in acetonitrile. 10 μL of the study sample and calibration curve, quality control, and blank whole blood samples were aliquoted in 96 deep well plates for processing. 10 μL of Milli-Q water was added to all the samples and briefly vortexed to initiate complete hemolysis. 10 μL of 20 mM dithiothreitol (DTT) was added to all the samples and incubated for 30 minutes at 37° C. The addition of DTT enhanced the recovery of ARM compounds of formula (I) from the biological matrix. 300 μL of working internal standard solution was added to all samples except total blank and wash samples, where 300 μL of acetonitrile was added. All the samples were vortex mixed for 5 minutes, followed by centrifugation at 4000 rpm for 10 minutes at 4° C. Supernatants were transferred to the loading plate and injected 3 μL to LC-MS/MS system for analysis.

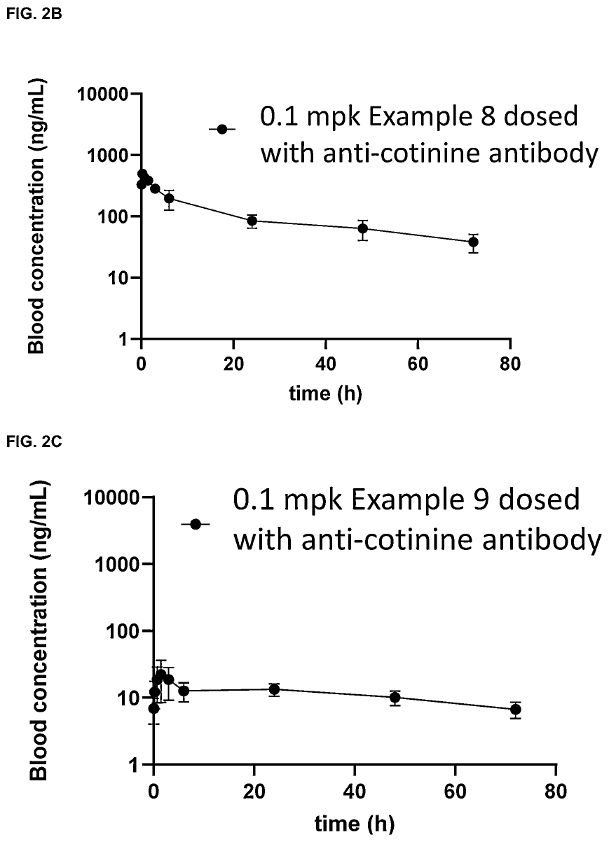

The results are shown in FIGS. 2A-2C. The results demonstrate that dosing the ARM compound of Formula (I) in the presence of anti-cotinine antibody extends the half-life of the compound of Formula (I).

SEQUENCE LISTINGS

Heavy chain CDR1 amino acid sequence
SEQ ID NO: 1
NYWMS
Heavy chain CDR2 amino acid sequence
SEQ ID NO: 2
DIHGNRGFNYHASWAKG
Heavy chain CDR3 amino acid sequence
SEQ ID NO: 3
ADDSGSHDI
Light chain CDR1 amino acid sequence
SEQ ID NO: 4
QSSQSVYSAKLS
Light chain CDR2 amino acid sequence
SEQ ID NO: 5
YGSTLAS
Light chain CDR3 amino acid sequence
SEQ ID NO: 6
QGTFYGPDWYFA
Variable heavy chain amino acid sequence
SEQ ID NO: 7
EVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIW
GQGTLVTVSS
Variable light chain amino acid sequence
SEQ ID NO: 8
DIQMTQSPSSLSASVGDRVTITCQSSQSVYSAKLSWYQQKPGKAPKLLIYYGSTLASGVPSRFS
GSGSGTQFTLTISSLQPEDFATYYCQGTFYGPDWYFAFGGGTKVEIK
Heavy chain amino acid sequence
SEQ ID NO: 9
EVQLVESGGGLVQPGGSLRLSCAASGFTFSNYWMSWVRQAPGKGLEWVGDIHGNRGFNYH
ASWAKGRFTVSRSKNTLYLQMNSLRAEDTAVYYCAKADDSGSHDIWGQGTLVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPDVFLFPPKPKDT
LMISRTPEVTCVVVDVSHEDPEVKFNWYVDGVEVHNAKTKPREEQYNSTYRVVSVLTVLHQD
WLNGKEYKCKVSNKALPAPEEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGK
Light chain amino acid sequence
SEQ ID NO: 10
DIQMTQSPSSLSASVGDRVTITCQSSQSVYSAKLSWYQQKPGKAPKLLIYYGST
LASGVPSRFSGSGSGTQFTLTISSLQPEDFATYYCQGTFYGPDWYFAFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
Heavy chain amino acid sequence
SEQ ID NO: 11
QQQLVESGGR LVTPGGSLTL TCTASGFSLN NYWMSWVRQA PGKGLEWIGD
IHGNRGFNYH ASWAKGRFTV SRTSTTVDLR MTSLTTEDTA IYFCARADDS GSHDIWGPGT
LVTVSSASTK GPSVFPLAPS SKSTSGGTAA LGCLVKDYFP EPVTVSWNSG ALTSGVHTFP
AVLQSSGLYS LSSVVTVPSS SLGTQTYICN VNHKPSNTKV DKKVEPKSCD KTHTCPPCPA
PELLGGPDVF LFPPKPKDTL MISRTPEVTC VVVDVSHEDP EVKFNWYVDG VEVHNAKTKP
REEQYNSTYR VVSVLTVLHQ DWLNGKEYKC KVSNKALPAP EEKTISKAKG QPREPQVYTL
PPSRDELTKN QVSLTCLVKG FYPSDIAVEW ESNGQPENNY KTTPPVLDSD GSFFLYSKLT
VDKSRWQQGN VFSCSVMHEA LHNHYTQKSL SLSPGK
Light chain amino acid sequence
SEQ ID NO: 12
ELDLTQTPSPVSAAVGDTVTINCQSSQSVYSAKLSWYQQKPGQPPKLLIYYGSTLASGVPSRF
KGSGSGTQFSLTISDVQCADAATYYCQGTYYGPDWYFAFGGGTEVVVKRTVAAPSVFIFPPSD
EQLKSGTASVVCLLNNFYPREAKVQWKVDNALQSGNSQESVTEQDSKDSTYSLSSTLTLSKAD
YEKHKVYACEVTHQGLSSPVTKSFNRGEC
Heavy chain amino acid sequence
SEQ ID NO: 13
QQQLVESGGRLVTPGGSLTLTCTASGFSLNNYWMSWVRQAPGKGLEWIGDIHGNRGFNYHA
SWAKGRFTVSRTSTTVDLRMTSLTTEDTAIYFCARADDSGSHDIWGPGTLVTVSSAKTTAPSV
YPLAPVCGDTTGSSVTLGCLVKGYFPEPVTLTWNSGSLSSGVHTFPAVLQSDLYTLSSSVTVT
SSTWPSQSITCNVAHPASSTKVDKKIEPRGPTIKPCPPCKCPAPNLLGGPSVFIFPPKIKDVLMIS
LSPMVTCVVVDVSEDDPDVQISWFVNNVEVLTAQTQTHREDYNSTLRVVSALPIQHQDWMSG
KEFKCKVNNKALPAPIERTISKPKGSVRAPQVYVLPPPEEEMTKKQVTLTCMVTDFMPEDIYVE
WTNNGKTELNYKNTEPVLDSDGSYFMYSKLRVEKKNWVERNSYSCSVVHEGLHNHHTTKSFS
RTPGK
Light chain amino acid sequence
SEQ ID NO: 14
ELDLTQTPSPVSAAVGDTVTINCQSSQSVYSAKLSWYQQKPGQPPKLLIYYGSTLASGVPSRF
KGSGSGTQFSLTISDVQCADAATYYCQGTYYGPDWYFAFGGGTEVVVKRADAAPTVSIFPPSS
EQLTSGGASVVCFLNNFYPKDINVKWKIDGSERQNGVLNSWTDQDSKDSTYSMSSTLTLTKDE
YERHNSYTCEATHKTSTSPIVKSFNRNEC

1. A compound of Formula (I): 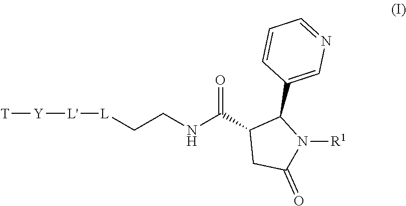
or a pharmaceutically acceptable salt thereof,
wherein:
T is 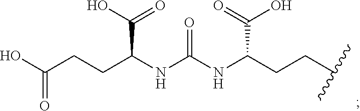 [SMILES: *CC[C@H](NC(=O)N[C@@H](CCC(=O)O)C(=O)O)C(=O)O]
R¹ is C₁₋₄ alkyl or C₃₋₆ cycloalkyl;
L′ is a bond, 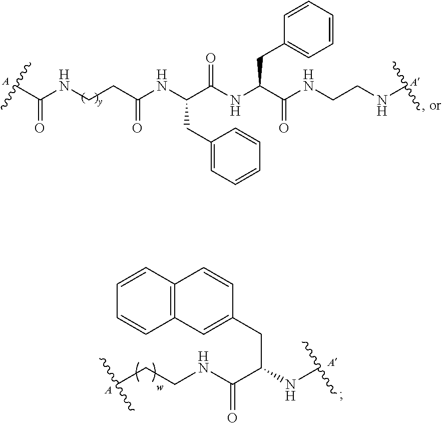
y is an integer of 1 to 9;
w is an integer of 0 to 5;
L is a divalent linker of Formula (L-a), (L-e), or (L-p): 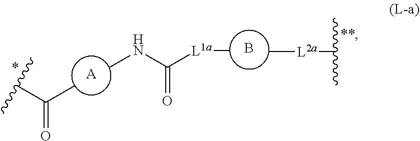
or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—; 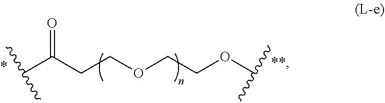
wherein n is an integer of 3 to 50; or 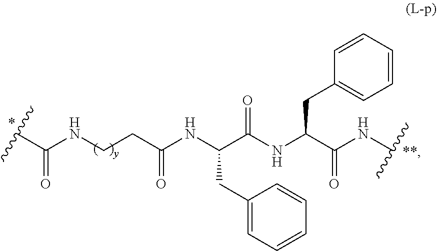
wherein y is an integer of 1 to 9;
wherein each 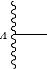
represents a covalent bond to the Y group of Formula (I), or when Y is a bond, a covalent bond to the T group of Formula (I), and each 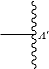
represents a covalent bond to the L group of Formula (I); and
wherein each 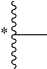
represents a covalent bond to the L′ group of Formula (I), or when L′ is a bond, a covalent bond to the Y group of Formula (I), or when both L′ and Y are a bond, a covalent bond to the T group of Formula (I), and each 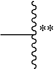
represents a covalent bond to the methylene group of Formula (I); and
Y is a bond or a divalent spacer moiety of one to twelve atoms in length.

2. The compound of claim 1, or a pharmaceutically acceptable salt thereof, wherein R¹ is —CH₃.

3. The compound of claim 1, or a pharmaceutically acceptable salt thereof, wherein L is a divalent linker of Formula (L-a-i): 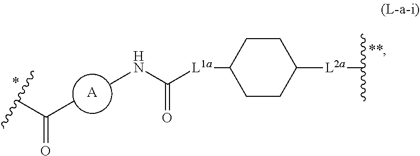
or a stereoisomer thereof,
wherein Ring A, L^{1a}, L^{2a}, 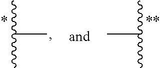
are as defined for Formula (L-a).

4. The compound of claim 1, or a pharmaceutically acceptable salt thereof, wherein L is a divalent linker of Formula (L-a-ii): 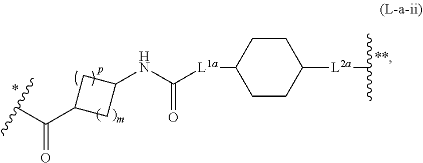
or a stereoisomer thereof,
wherein L^{1a}, L^{2a}, 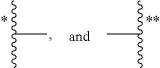
are as defined for Formula (L-a); p is 1 or 2; and m is 1 or 2.

5. The compound of claim 1, or a pharmaceutically acceptable salt thereof, wherein L is a divalent linker of Formula (L-a-iii): 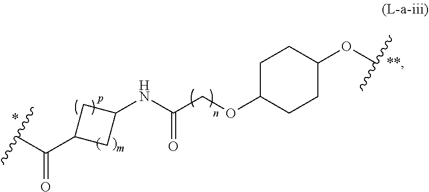
or a stereoisomer thereof,
wherein p is 1 or 2; m is 1 or 2; n is 1, 2, or 3; and 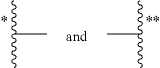
are as defined for Formula (L-a).

6. The compound of claim 1, or a pharmaceutically acceptable salt thereof, wherein L is a divalent linker of Formula (L-a) selected from the group consisting of:

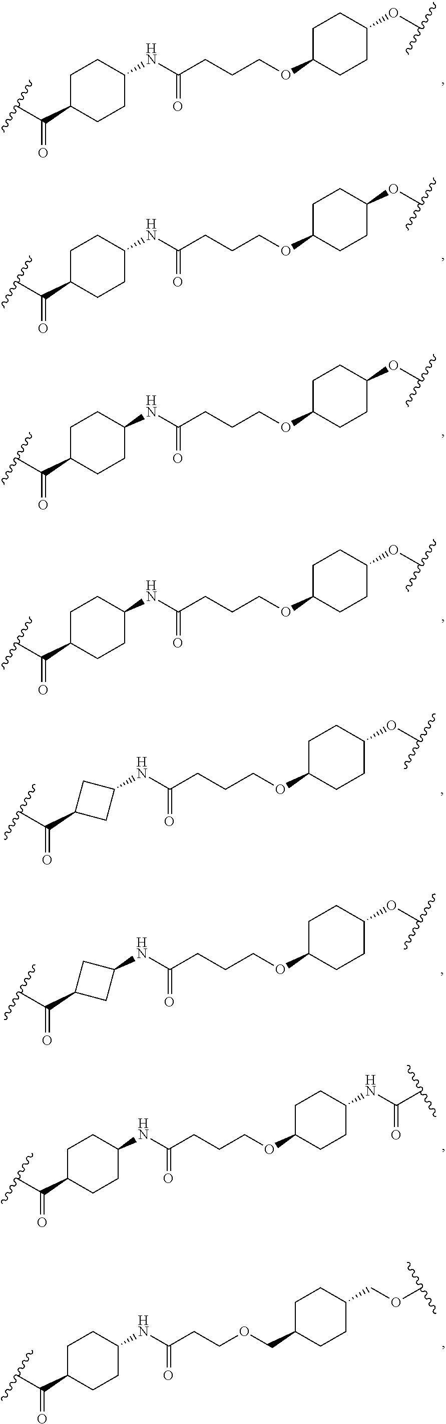

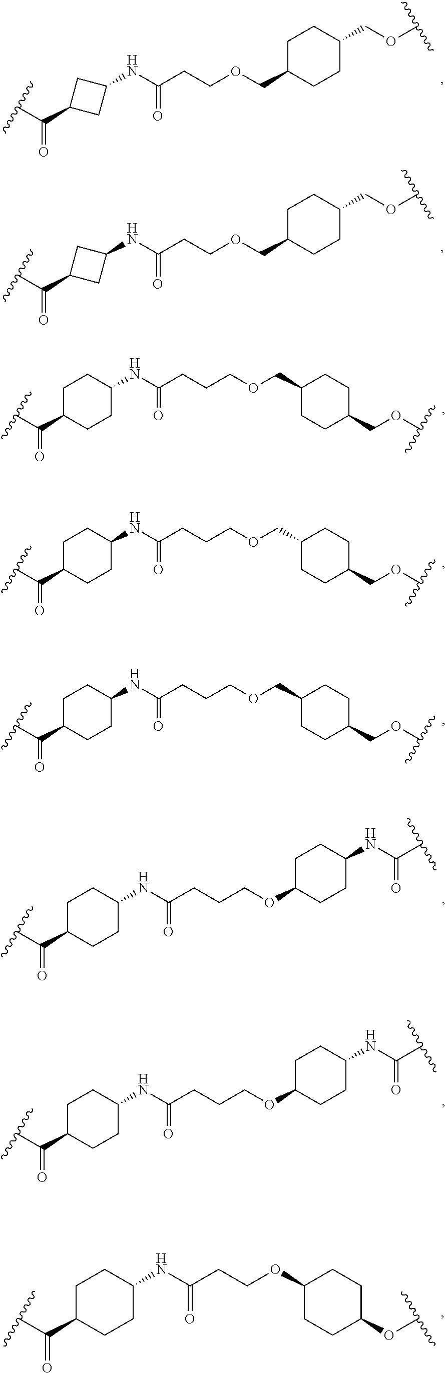

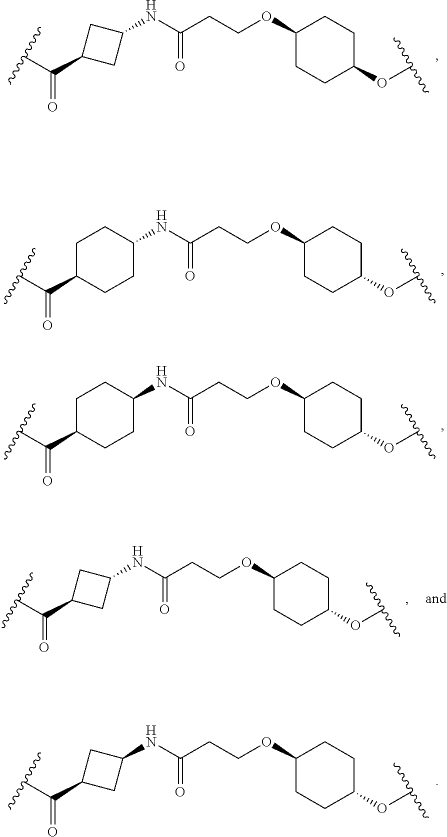 [SMILES: *O[C@H]1CC[C@@H](OCCC(=O)N[C@H]2C[C@H](C(*)=O)C2)CC1 ; *O[C@H]1CC[C@H](OCCC(=O)N[C@H]2CC[C@H](C(*)=O)CC2)CC1 ; *O[C@H]1CC[C@H](OCCC(=O)N[C@H]2CC[C@@H](C(*)=O)CC2)CC1 ; *O[C@H]1CC[C@H](OCCC(=O)N[C@H]2C[C@H](C(*)=O)C2)CC1 ; *O[C@H]1CC[C@H](OCCC(=O)N[C@H]2C[C@@H](C(*)=O)C2)CC1]

7. The compound of claim 3, or a pharmaceutically acceptable salt thereof, wherein Y is selected from a bond; —NH—; —(C₁₋₁₂ alkylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene; or —(C₂₋₁₂ alkenylene)-, wherein 1, 2, or 3 methylene units are replaced with —O—, —NH—, —C(O)—, —NHC(O)—, —C(O)NH—, —(C₃₋₆ cycloalkylene)-, —(C₃₋₆ cycloalkenylene)-, 3- to 6-membered heterocycloalkylene, arylene, or heteroarylene.

8. (canceled)

9. The compound of claim 6, or a pharmaceutically acceptable salt thereof, wherein Y is selected from the group consisting of:

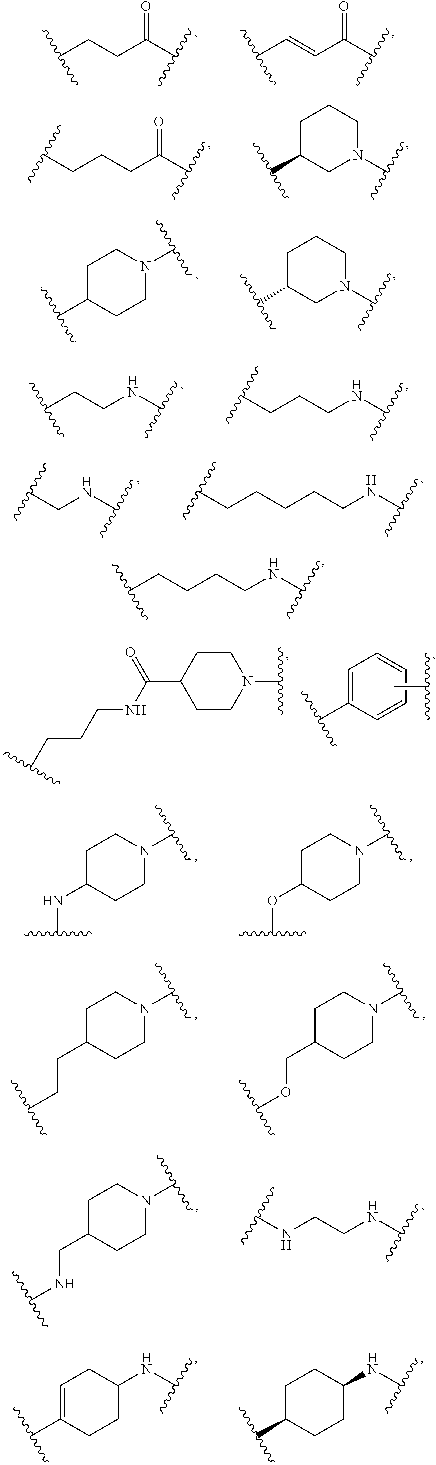

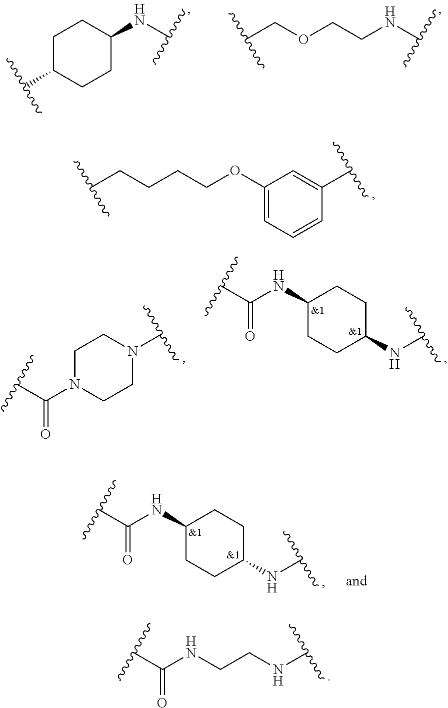

10. The compound of claim 1, wherein the compound is a compound in Table 1 or a pharmaceutically acceptable salt thereof.

11. A compound selected from the group consisting of: 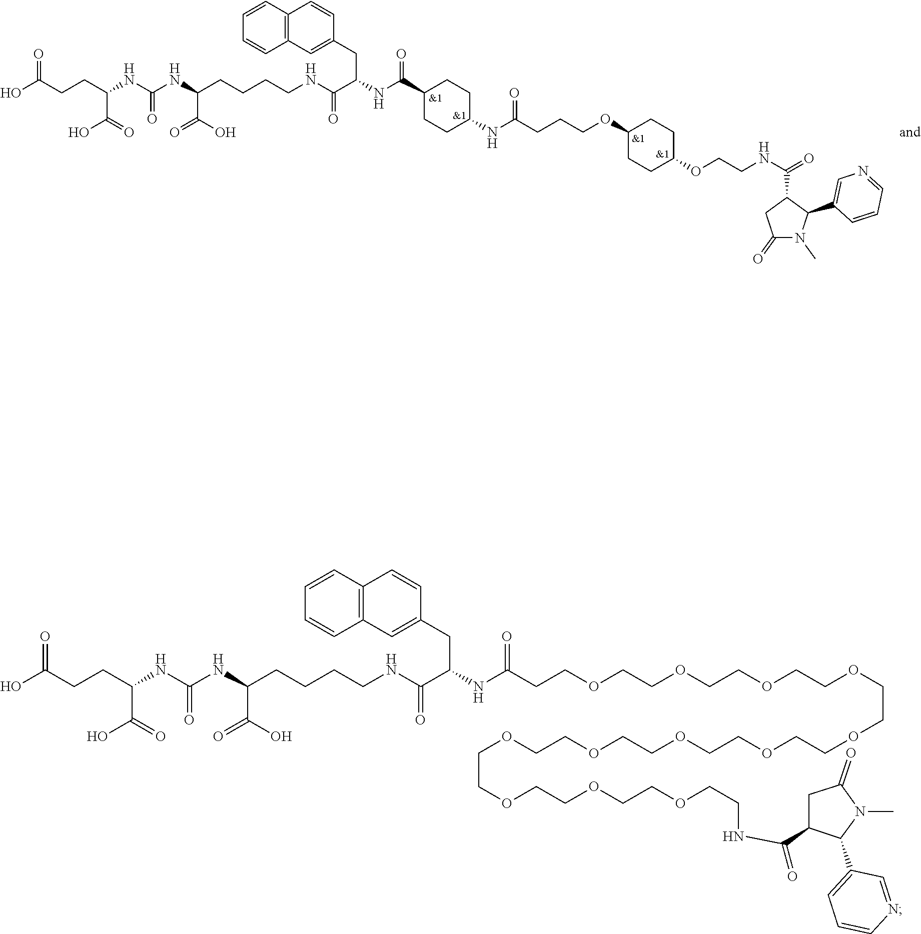 [SMILES: CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1 ; CN1C(=O)C[C@H](C(=O)NCCOCCOCCOCCOCCOCCOCCOCCOCCOCCOCCOCCOCCC(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1]
or a pharmaceutically acceptable salt thereof.

12. A method of treating and/or preventing a disease or disorder in a patient in need thereof, the method comprising: administering to the patient a therapeutically effective amount of the compound of claim 1 and an anti-cotinine antibody, or antigen-binding fragment thereof, wherein the disease or disorder is selected from a cancer.

13. The method of claim 12, wherein the disease or disorder is mediated by PSMA and/or is associated with PSMA-positive pathogenic cells.

14. The method of claim 12, wherein the disease is a cancer that is a solid tumor.

15. The method of claim 12, wherein the disease or disorder is a cancer selected from leukemia, lymphoma, lung cancer, hepatocellular carcinoma (HCC), colorectal cancer (CRC), cervical cancer, head and neck cancer, pancreatic cancer, prostate cancer, ovarian cancer, endometrial cancer, renal cell cancer, bladder cancer, or breast cancer.

16. The method of claim 15, wherein the cancer is metastatic castration-resistant prostate cancer (mCRPC).

17. (canceled)

18. (canceled)

19. A method of increasing antibody-dependent cell cytotoxicity (ADCC) of PSMA-expressing cells, the method comprising: contacting the cells with an effective amount of the compound of claim 1 and an anti-cotinine antibody, or antigen-binding fragment thereof, wherein the PSMA-binding moiety of the compound binds the PSMA expressed on the cells.

20. A method of depleting PSMA-expressing cells, the method comprising: contacting the cells with an effective amount of the compound of claim 1 and an anti-cotinine antibody, or antigen-binding fragment thereof, wherein the PSMA-binding moiety of the compound binds the PSMA expressed on the cells.

21. (canceled)

22. The method of claim 15 wherein the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

23. The method of claim 15, wherein the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8.

24. (canceled)

25. (canceled)

26. The method of claim 15, wherein the anti-cotinine antibody has a heavy chain comprising SEQ ID NO: 9 and a light chain comprising SEQ ID NO: 10.

27. A combination comprising the compound of claim 1 and an anti-cotinine antibody, or antigen-binding fragment thereof.

28. The combination of claim 27, wherein the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

29-32. (canceled)


SEQUENCE LISTING (14 sequences)

SEQ ID NO: 1 - protein, 5 aa, synthetic construct
  REGION 1..5: note = Synthetic Sequence
NYWMS

SEQ ID NO: 2 - protein, 17 aa, synthetic construct
  REGION 1..17: note = Synthetic Sequence
DIHGNRGFNYHASWAKG

SEQ ID NO: 3 - protein, 9 aa, synthetic construct
  REGION 1..9: note = Synthetic Sequence
ADDSGSHDI

SEQ ID NO: 4 - protein, 12 aa, synthetic construct
  REGION 1..12: note = Synthetic Sequence
QSSQSVYSAKLS

SEQ ID NO: 5 - protein, 7 aa, synthetic construct
  REGION 1..7: note = Synthetic Sequence
YGSTLAS

SEQ ID NO: 6 - protein, 12 aa, synthetic construct
  REGION 1..12: note = Synthetic Sequence
QGTFYGPDWYFA

SEQ ID NO: 7 - protein, 116 aa, synthetic construct
  REGION 1..116: note = Synthetic Sequence
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 8 - protein, 111 aa, synthetic construct
  REGION 1..111: note = Synthetic Sequence
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 9 - protein, 446 aa, synthetic construct
  REGION 1..446: note = Synthetic Sequence
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
  REGION 1..218: note = Synthetic Sequence
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
  REGION 1..446: note = Synthetic Sequence
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
  REGION 1..218: note = Synthetic Sequence
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
  REGION 1..446: note = Synthetic Sequence
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 218 aa, synthetic construct
  REGION 1..218: note = Synthetic Sequence
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC